(19) United States
(12) Patent Application Publication
(10) Pub. No.: US 2025/0313853 A1
(43) Pub. Date: Oct. 9, 2025

(54) USE OF ENGINEERED JURONA VIRUS (JURV) AS AN ONCOLYTIC VIRUS PLATFORM FOR HUMAN CANCERS

(71) Applicant: BIOVENTURES, LLC, Little Rock, AR (US)

(72) Inventor: Bolni NAGALO, Little Rock, AR (US)

(21) Appl. No.: 18/867,764

(22) PCT Filed: May 19, 2023
(86) PCT No.: PCT/US2023/067252

§ 371 Date: Nov. 20, 2024

Related U.S. Application Data
(60) Provisional application No. 63/344,395, filed on May 20, 2022.

(51) Int. Cl. C12N 15/86; A61K 35/766

(52) U.S. Cl. CPC C12N 15/86; A61K 35/766; C12N 2760/20232; C12N 2760/20243; C12N 2760/20252

(57) ABSTRACT

The present disclosure provides compositions comprising recombinant polynucleotides encoding Jurona virus, infectious particles, pharmaceutical compositions, and cells comprising the same, and methods and systems for making recombinant Jurona virus.

### CROSS-REFERENCE TO RELATED APPLICATIONS

This application claims priority to U.S. Provisional Application No. 63/344,395 filed on May 20, 2022, the contents of which are incorporated by reference in their entirety.

### STATEMENT REGARDING FEDERALLY SPONSORED RESEARCH

This invention was made with government support under CA234324 awarded by the National Cancer Institute. The government has certain rights in the invention.

### SEQUENCE LISTING

A Sequence Listing accompanies this application and is submitted as an ASCII text file of the sequence listing named “169852_00112_Sequence_Listing.xml” which is 110,996 bytes in size and was created on May 3, 2023. The sequence listing is electronically submitted via EFS-Web with the application and is incorporated herein by reference in its entirety.

### BACKGROUND

Oncolytic viruses, viruses that preferentially infect and kill cancer cells, represent a promising advance in the treatment of cancer. Currently, several viral platforms are being studied as oncolytic viruses including vesicular stomatitis virus (VSV). VSV is a widely studied vector and has been advanced into early phase clinical studies. However, concerns for hepatic and neurological toxicities could hinder its clinical deployment for the treatment of human cancers. Other vesiculovirus vectors have been developed in recent years, yet most have failed to achieve the level of potency of VSV in preclinical models of human cancers. Thus, there remains a need in the art for novel oncolytic viruses.

### SUMMARY

In one aspect of the current disclosure, constructs and compositions comprising the constructs including a promoter operably linked to a polynucleotide encoding a Jurona virus genome or the sense strand copy of the Jurona virus genome are provided. The Jurona virus has a negative sense single stranded RNA genome such that the constructs will comprise the full-length antisense genome and allow production of a negative sense viral genome and production of infectious virus when transfected into mammalian cells. The polynucleotide encoding the Jurona virus genome may include SEQ ID NOs: 1-5 and optionally a leader sequence of SEQ ID NO: 6 and/or a trailer sequence of SEQ ID NO: 7. The polynucleotide encoding the Jurona virus genome may further comprise at least one of SEQ ID NOs: 8-11, 21, and 22 as intergenic regions. In some embodiments, the polynucleotide encoding the Jurona virus genome is or comprises SEQ ID NO: 12 (JURV-XN-2). The polynucleotide encoding the Jurona virus genome may further comprise a heterologous polynucleotide capable of encoding a polypeptide not natively associated with Jurona virus. In some embodiments, the polypeptide is a reporter polypeptide and may be encoded by the polynucleotide is SEQ ID NO: 13 (JURV-eGFP).

In another aspect of the current disclosure, further compositions or constructs are provided. Some of the constructs comprise a codon-optimized polynucleotide encoding at least one Jurona virus protein selected from the group consisting of glycoprotein (G), nucleoprotein (N), phosphoprotein (P), RNA-directed RNA polymerase L protein (L), and matrix protein (M) operably linked to a promoter for expression in mammalian cells. The polynucleotide encoding the G protein may comprise SEQ ID NO: 4, the polynucleotide encoding the N protein may comprise SEQ ID NO: 1, the polynucleotide encoding the P protein may comprise SEQ ID NO: 2, the polynucleotide encoding the M protein may comprise SEQ ID NO: 3, the polynucleotide encoding the L protein may comprise SEQ ID NO: 5, and the compositions may comprise a plasmid and more than one polynucleotide encoding a viral polypeptide may be encoded on a single construct. Such constructs may be contained in the genome of a cell or can be transiently transfected into cells to produce the viral proteins and allow for packaging and production of infectious virus. Thus, cells comprising the constructs described herein are also provided.

In another aspect of the current disclosure infectious particles are provided. The infectious particles may comprise a Jurona virus genome comprising a negative sense RNA of SEQ ID NO: 12. The infectious particles may be made via the method provided in the Examples and may use the constructs and cells provided here to generate recombinant infectious virus particles.

In another aspect of the current disclosure, further infectious particles are provided. The infectious particles are made by transfecting cells with the composition comprising a codon optimized polynucleotide encoding at least one Jurona virus protein selected from the group consisting of G, N, P, L, and M operably linked to a promoter for expression in mammalian cells. The polynucleotide encoding the G protein may comprise SEQ ID NO: 4, the polynucleotide encoding the N protein may comprise SEQ ID NO: 1, the polynucleotide encoding the P protein may comprise SEQ ID NO: 2, the polynucleotide encoding the M protein may comprise SEQ ID NO: 3, the polynucleotide encoding the L protein may comprise SEQ ID NO: 5, and the compositions may comprise a plasmid. The cells are also transfected with a construct encoding the negative sense genomic copy of the Jurona virus operably linked to a promoter capable of generating the genomic copy of the virus.

In another aspect of the current disclosure, pharmaceutical compositions are provided. The pharmaceutical compositions comprise an infectious particle comprising a Jurona virus genome comprising a negative sense RNA from SEQ ID NO: 12 or having at least 95% sequence identity to SEQ ID NO: 12 and a pharmaceutically acceptable carrier or excipient. In some embodiments, the pharmaceutical compositions comprise an infectious particle made by transfecting cells with the composition comprising a codon optimized polynucleotide encoding at least one Jurona virus protein selected from the group consisting of G, N, P, L, and M operably linked to a promoter for expression in mammalian cells and a pharmaceutically acceptable carrier or excipient.

In other aspects of the current disclosure, methods for treating a cell proliferative disease or disorder in a subject in need thereof are provided. The methods comprise administering a pharmaceutical composition comprising an infectious particle comprising a Jurona virus genome comprising a negative sense RNA of SEQ ID NO: 12 or a sequence having at least 95% identity to SEQ ID NO: 12 and a pharmaceutically acceptable carrier or excipient to a subject to treat the cell proliferative disease or disorder. The infectious particle may be made by transfecting cells with the composition comprising a codon optimized polynucleotide encoding at least one Jurona virus protein selected from the group consisting of G, N, P, L, and M operably linked to a promoter for expression in mammalian cells and a pharmaceutically acceptable carrier or excipient. The cell proliferative disease or disorder can be cancer and can be selected from hepatocellular carcinoma, liver bile duct carcinoma, breast cancer, colorectal cancer, prostate cancer, and reticulum sarcoma. The breast cancer may be HER2-negative. The cancer may be local or metastatic. The subject may have a suppressed immune system. The methods may further comprise administering to the subject an immunotherapy which may be a checkpoint inhibitor therapy. The checkpoint inhibitor therapy may be selected from the group consisting of inhibitors of PD-1, inhibitors of PD-L1, inhibitors of CTLA-4, and inhibitors of LAG-3.

In another aspect of the current disclosure, cells are provided. The cells comprise a promoter operably linked to a polynucleotide encoding a Jurona virus genome and allowing production of a negative sense viral genome when transfected into mammalian cells. In some embodiments, the promoter is a T7 promoter. The cells may comprise a codon optimized polynucleotide encoding at least one Jurona virus protein selected from the group consisting of G, N, P, L, and M operably linked to a promoter for expression in mammalian cells. The cells may further or alternatively comprise an infectious particle comprising a Jurona virus genome comprising a negative sense RNA of SEQ ID NO: 12 and the infectious particle may be made by transfecting cells with the composition comprising a codon optimized polynucleotide encoding at least one Jurona virus protein selected from the group consisting of G, N, P, L, and M operably linked to a promoter for expression in mammalian cells and the composition encoding the full length genome and capable of generating the negative sense genomic RNA.

In another aspect of the current disclosure, methods of generating recombinant Jurona virus are provided. The methods comprise introducing at least one composition comprising a promoter operably linked to a polynucleotide encoding a Jurona virus genome and allowing production of a negative sense viral genome when transfected into mammalian cells into a cell; allowing the cell to express one or more Jurona virus proteins selected from the group consisting of G, M, N, L and P; incubating the cells for a sufficient time to generate recombinant Jurona virus; and harvesting virus produced by the cells. The cell may comprise a promoter operably linked to a polynucleotide encoding a Jurona virus genome and allowing production of a negative sense viral genome when transfected into mammalian cells. The one or more Jurona virus proteins may comprise Jurona virus N, P, and L proteins. The one or more Jurona virus proteins may be encoded by one or more polynucleotides comprising SEQ ID NO: 1, 2, or 5. The promoter may be T7 promoter, and the cell may comprise T7 RNA polymerase. In some embodiments, the cell is a BHK-21 cell, a Vero cell, or a HEK-293 cell. The introduced composition(s) may additionally comprise a heterologous polynucleotide encoding a protein not natively associated with a Jurona virus.

In another aspect of the current disclosure, systems for generating a recombinant Jurona virus are provided. The systems comprise: a) one or more vectors comprising polynucleotides encoding at least three Jurona virus proteins selected from the group consisting of G, N, P, L, and M each operably linked to a promoter to allow for expression of the at least three proteins in a mammalian cell; b) a vector comprising a polynucleotide encoding a negative sense Jurona virus genome operably linked to a promoter to allow production of the negative sense Jurona virus genome in a mammalian cell, and may further comprise: (c) mammalian cells capable of expressing the Jurona virus proteins of (a) and the negative sense Jurona virus genome of (b) to produce the recombinant Jurona virus. The cells may comprise T7 RNA polymerase, and/or at least one of the promoters may be a T7 promoter. In some embodiments, the cells are BHK-1 cells, Vero cells, or HEK-293 cells. The one or more vectors may comprise polynucleotides encoding at least the Jurona virus N, P, and L proteins operably linked to a promoter. The polynucleotides encoding the at least three Jurona virus proteins may be codon-optimized for expression in mammalian cells and may comprise any one of SEQ ID NOs: 1-5. The vector may comprise a polynucleotide encoding the negative sense Jurona virus genome described herein. The one or more vectors encoding at least three Jurona virus proteins may include the compositions provided herein.

In other aspects of the current disclosure, kits are provided. In some embodiments, the kits comprise a composition with a promoter operably linked to a polynucleotide encoding a Jurona virus genome and allowing production of a negative sense viral genome when transfected into mammalian cells. The promoter may be a T7 promoter. The polynucleotide encoding the Jurona virus genome may comprise SEQ ID NOs: 1-5, a leader sequence of SEQ ID NO: 6, a trailer sequence of SEQ ID NO: 7, and/or at least one of SEQ ID NOs: 8-11, 21, and 22 as intergenic regions. In some embodiments, the polynucleotide encoding the Jurona virus genome comprises SEQ ID NO: 12 (JURV-XN-2). The polynucleotide encoding the Jurona virus genome may further comprise a heterologous polynucleotide capable of encoding a polypeptide not natively associated with Jurona virus, and the polypeptide may be a reporter polypeptide which may be encoded by the polynucleotide of SEQ ID NO: 13 (JURV-eGFP). The kits can alternatively or additionally comprise any of the compositions or infectious particles disclosed herein. The kits may further comprise an immunotherapy which may be a checkpoint inhibitor therapy. The checkpoint inhibitor therapy may be selected from the group consisting of inhibitors of PD-1, inhibitors of PD-L1, inhibitors of CTLA-4, and inhibitors of LAG-3. The kits may further comprise an inhibitor of IFN-α and/or a receptor tyrosine kinase inhibitor which may be pazopanib.

### BRIEF DESCRIPTION OF THE DRAWINGS

FIG. 1 shows a schematic of the organization and structure of vesiculoviruses (FIG. 1A) and vesiculovirus genomes (FIG. 1).

FIG. 2 shows a schematic of (A) the method used to derive a laboratory adapted Jurona virus clone and (B) the process of developing a reverse genetics-based method of generating recombinant Jurona virus.

FIG. 3 shows (A) a schematic of the genome of Jurona virus, (B) cultured tumor cells were infected with JURV at multiplicity of infection (MOI) 0.1, 1, and 10 and the viability of cells was measured. (C) as in (B), cancer cells were infected with Morreton virus (MORV) at MOI of 0.1, 1, and 10 and cell viability was observed. (D) cancer cells were infected with vesicular stomatitis virus (VSV) at MOI of 0.1, 1, and 10 and cell viability was observed. The data demonstrates that JURV has oncolytic function. (E) shows that JURV can replicate in a variety of tumor cells and Vero cells.

FIG. 4 shows changes in bodyweight in animals subjected to intranasal (A) administration of PBS vehicle control or low dose 1×10⁷ tissue culture infected dose 50 (TCID₅₀) units and high dose 1×10⁸ TCID₅₀ JURV. Panel (B) shows brain tissue from 1×10⁸ TCID₅₀ infected animals at day 3 post-infection (short-term toxicity) exhibiting relatively normal histological features. Panel (C) shows changes in bodyweight in animals subjected to intravenous administration of PBS vehicle control, low, or high dose of JURV. Panel (D) shows histology of liver taken from 1×10⁸ TCID₅₀ infected animals at day 3-post-infection (short-term toxicity) exhibiting relatively normal histology. Panel (E) shows total white blood cell (WBC) counts in animals inoculated with the vehicle or JURV at low or high concentrations intranasally or intravenously demonstrating a modest reduction in total WBC count in infected animals. Panel (F) shows a reduction in total lymphocyte count in infected animals. Panel (G) shows similar monocyte counts in infected animals compared to controls. Likewise, panel (H) shows no change in neutrophil counts in infected animals.

FIG. 5 shows the tumor volume and survival of animals implanted with Hepa 1-6, EMT6, CT26, and RM-1 tumor cells.

FIG. 6 shows the Anti-tumor activity of JURV against localized and untreated distant mouse hepatocellular carcinoma (HCC). FIGS. 6 A & B show intratumoral injections of JURV (1×10⁷ TCID₅₀, once weekly for 3 weeks) and immune checkpoint blockade (Anti-PD-1, 10 mg/kg, twice weekly for 3 weeks) triggered tumor regressions in subcutaneous syngeneic HCC mice (Hepa 1-6). However, the combination JURV+Anti-PD-1 was not superior to JURV as monotherapy. Moreover, (FIG. 6 B), in mice bearing tumors on the right and left flanks in which the right flanks were injected, prominent tumor growth delay was observed in both the right (injected flank) and the left flanks (non-injected), highlighting the ability of JURV to trigger an anti-tumor immune response capable of targeting and killing localized tumors and distant metastases. (FIG. 6 C-L), While weekly doses of JURV f were associated with a significant decrease in F4/80− (C) tumor-infiltrating lymphocytes (TILs) cells and increased NK (D), NKT (E) TILs, and M2-like (F) macrophages compared to control (PBS), anti-PD-1 and dual flank (Left non-injected flank). PD-1 blockage significantly correlated with the intratumoral accumulation of cytotoxic CD8+ T cells, mainly CD8+ Ki67+ (G), CD8+ PD-1 (H), and CD8+ CD44+ (I) compared to control (PBS), JURV alone, and dual flank (Left). The combination of JURV+anti-PD-1 therapy profoundly modulates both adaptive and innate immunity, manifested by an increase in F4/80− CD4+ (J) and CD4+ PD-1+ (K), CD11b+ (L), CD11b−(M) and decrease in CD8+ PD-1+ (N), NK compared to JURV, anti-PD-1, and dual flank (Left). In addition, there was also a prominent decrease in the TILs macrophages (anti-PD-1 vs. PBS), M1 (O) (anti-PD-1 vs. PBS), and M2-like (anti-PD-1 vs. JURV) in the anti-PD-1 treated group.

FIG. 7 Panel (A) shows representative bioluminescent imaging of mice implanted with luminescent Hep3B tumor cells, the summary results of which are displayed graphically in panel (B), with the individual tumor curves displayed in panel (C) and the survival curve displayed in (D).

FIG. 8 shows a plasmid map of JURV-XN-2 comprising SEQ ID NO: 14.

FIG. 9 shows a plasmid map JURV-GFP comprising SEQ ID NO: 15.

FIG. 10 shows plasmid maps for (A) JURV-N(SEQ ID NO: 16), (B) JURV-P (SEQ ID NO: 17), (C) JURV-M (SEQ ID NO: 18), (D) JURV-G (SEQ ID NO: 19), and (E) JURV-L (SEQ ID NO: 20).

FIG. 11 shows schematic representation of the antigenomic structure of JURV (A), VSV (B), and MORV (C). The antigenome of JURV is typical of that of Rhabdovirus and consist of five major genes from the 3′ to 5′ antigenomic direction: nucleoprotein (JURV-N), phosphoprotein (JURV-P), matrix (JURV-M), glycoprotein (JURV-G), polymerase (JURV-L). Schematic representation of the 3′ to 5′ antigenomic organization of a) attenuated Jurona virus (JURV), b) Wild-type vesicular stomatitis virus (VSV), and c) Morreton virus (MORV). The gene junctions' intergenic region (IGR) differed between the three viruses. (A) The antigenome organization of JURV consists of (3′ to 5′) of a leader (Le) sequence followed by approximately 10,993 bases comprising five structural genes (JURV-N, JURV-P, JURV-M, JURV-G, and JURV-L) separated by a highly conserved intergenic region (IGR) and a trailer sequence (Tr).

FIG. 12 shows oncolytic JURV is effective in inducing oncolysis in HCC cell lines. Monolayers of human HCC, HEP3B (A), PLC (B), HuH7(C) and murine HCC, HEPA 1-6 (D) and RILWT(E) were seeded at a density of 1.5×10⁴/well in 96-well plates and infected with JURV, recombinant, Morreton virus (MORV) and recombinant vesicular stomatitis virus (VSV) at the indicated multiplicity of infection (MOI) of 10, 1 and 0.1. The percentage of cell viability was determined 72 hours post-infection using a colorimetric assay (MTS, Promega USA). The discontinued lines on the graphs indicate the cut-off percentage for resistance (>50% cell viability above the line) and sensitivity (<50% of cell viability, below the line). Data was collected from multiple replicates over three independent experiments. Bars indicate mean±SEM.

FIG. 13 shows toxicologic pathology examination and dose-dependent response to treatment with type interferon on the oncolytic activity of JURV. Effect of exogenous IFN-β on the outcome of JURV-induced tumor cell death. Infectious titers and MOI dependent responses to species-specific exogenous type I IFN-β on HEP3B and HEPA 1-6 cell lines used as tumor models (A) HEP3B and (B) HEPA 1-6 cell lines. Results are from three independent experiments and are plotted as mean±SEM. Intranasal (IN, brain) and intravenous (IV, liver and spleen) administration of 1×10⁷ or 1×10⁸ TCID₅₀ of JURV in non-tumor bearing mice.

FIG. 14 shows effects of low and high doses of oncolytic JURV on body weight and hemogram in mice. Non-tumor bearing female C57BL6/J (n=6/group; Strain #:000664) of 6-8 weeks of age were administered intranasally (IN) or intravenously (IV) single doses of 1×10⁷ TCID₅₀ or 1×10⁸ TCID₅₀ of JURV. Body weight was recorded twice a week in both the IN and IV cohorts to assess drug-related toxicity. Three mice per group in each cohort (IN &IV) were sacrificed three days post-infection, and blood, brain, and liver were harvested to assess the short-term toxicity. Changes in body weight and hematoxylin and eosin (H&E) staining (brain and liver) are shown for mice administered IN (A, B) and IV (C, D) in mice treated with low (1×10⁷ TCID₅₀) and high low (1×10⁸ TCID₅₀) doses of JURV. Complete blood count, including white blood cells (E), lymphocytes (F), monocytes (G), and neutrophils (H), assessment of JURV-induced toxicity.

FIG. 15 shows toxicoproteomic analysis of brain and liver tissues of mice injected with high doses of oncolytic JURV. Intranasal and intravenous administration of oncolytic JURV in mice causes changes in the expression of proteins associated with sensing RNA viruses and antiviral signaling pathways. Volcano plot of protein expression differences for the brain (A) and liver (C) of mice treated with PBS vs. 1×10⁸ TCID₅₀ of JURV. 3-D Pie slices of the numbers of differentially expressed proteins (DEPs) for the brain (B) and liver (D) of mice injected with PBS vs. 1×10⁸ TCID₅₀ of JURV. Heatmaps of the top 20 DEPs up or down regulated in the brain (E) and liver (F) of mice injected with PBS vs. 1×10⁸ TCID₅₀ of JURV. DEPs were determined using the limma-voom method. A fold-change |log FC|≥1 and a false discovery rate (FDR) of 0.055 were used as a cutoff. The log FC was computed using the difference between the mean of log 2-(JURV) and the mean of log 2(PBS), that is, mean of log 2(JURV)−mean of log 2(PBS). Graph showing top-scoring canonical pathways significantly enriched by treatment with 1×10⁸ TCID₅₀ of JURV in the brain (G) and liver (H).

FIG. 16 shows toxicoproteomic analysis of differentially expressed proteins in brain and liver tissues of mice injected with low doses of oncolytic JURV in healthy non-tumor bearing. Volcano plot of protein expression differences for the brain (A) and liver (C) of mice treated with PBS vs. 1×10⁷ TCID₅₀ of JURV. 3-D Pie slices of the numbers of differentially expressed proteins (DEPs) for the brain (B) and liver (D) of mice injected with PBS vs. 1×10⁷ TCID₅₀ of JURV. Heatmaps of the top 20 DEPs up or down regulated in the brain (C) and liver (D) of mice injected with PBS vs. 1×10⁷ TCID₅₀ of JURV. DEPs were determined using the limma-voom method. A fold-change |log FC|≥1 and a false discovery rate (FDR) of 0.055 were used as a cutoff. The log FC was computed using the difference between the mean of log 2(JURV) and the mean of log 2(PBS), that is, mean of log 2(JURV)−mean of log 2(PBS). Graph showing top-scoring canonical pathways significantly enriched by treatment with 1×10⁷ TCID₅₀ of JURV in the brain (E) and liver (F).

FIG. 17 shows evaluation of the anti-tumor efficacy of oncolytic JURV as monotherapy or combined with anti-PD-1 antibodies in murine HCC model. HEPA 1-6 cells were implanted into the right flanks of female C57BL6/J (Strain #:000664) (n=7/group; Jackson Laboratory). (A) Upon reaching 80-120 mm³, mice were administered 50 μL IT injections containing PBS (Vehicle), 1×10⁷ TCID₅₀ units of JURV, anti-PD-1 therapy, or combination JURV+anti-PD-1. Anti-PD-1 antibodies (10 mg/kg) were given intraperitoneally (IP) twice a week for three weeks. (B) In the treatment schedule, at day −14, tumor cells were subcutaneously implanted into the right flanks of mice, then PBS, JURV, anti-PD-1 antibodies (PD-1), JURV+anti-PD-1 was injected (inj.) into tumor-bearing mice at days 0, 7, and 14. Tumors were harvested at the end of the study for downstream analysis. (C) In the abscopal model (dual flanks), HEPA 1-6 cells (1×10⁶ cells/mouse) were first subcutaneously grafted into the right flanks and were categorized as “primary” tumors. Simultaneously, we performed distant HEPA 1-6 tumor grafts injection (1×10⁶ cells/mouse) into the left flanks of these mice. Mice in the dual flank group received 50 μL IT injections of 1×10⁷ TCID₅₀ units of JURV on their right flanks only once a week for three weeks. Data plotted as mean+/−SD, **p<0.001 ***p<0.0001. Area under tumor growth curves was compared by one-way ANOVA with Holm-Sidak correction for type I error. The day when we injected the first JURV or PBS into the mice is defined as day 0. Combination JURV+ anti-PD-1 therapy profoundly modulated tumor microenvironment as manifested by changes in the frequency of (D) CD8+ Ki67+, (E) CD8+PD-1+, (F) CD8+CD44+, (G) CD4+, (H) CD4+ PD-1+, (I) macrophages, (J) M1-like macrophages, (K) M2-like macrophages, and (L) NK cells. The sub-myeloid population representing macrophages are CD11b+F480+ cells, and the two markers used to segregate the M1 and M2 sub-populations are CD206 and I-A/I-E, known as major histocompatibility class II (MHCII) in mouse. The Bartlett test was used to test homogeneity of variance and normality. If the p value of the Bartlett test was no less than P=0.05, ANOVA and a two-sample t test were used to compare group means. If the P value of the Bartlett test was less than 0.05, the Kruskal-Wallis and Wilcoxon rank-sum tests were used to compare group means. The figures demonstrate the potential significant difference of the gated subsets in the CD45+ population, determined by the p value.

FIG. 18 shows changes in body weight and Survival of Hepa 1-6 tumor-bearing mice Treated with oncolytic JURV. Individual tumor volumes of HEPA 1-6 tumor-bearing mice between various groups in the (A) combination therapy study and (B) abscopal effect study. (C) Body weight of HEPA 1-6 tumor-bearing mice between various groups in the combination therapy study and abscopal effect study. Kaplan-Meier survival analysis of HEPA 1-6 tumor-bearing mice in the (D) combination therapy study (E) and abscopal effect study. The statistical significance of differences in the survival curves between the groups was evaluated using the log-rank (Mantel-Cox) test.

FIG. 19 shows anti-tumor efficacy of JURV on a panel of murine solid tumor models. Murine EMT6 (breast cancer), CT26 (colon cancer), and A20 (reticulum sarcoma) cells were implanted on the right flanks (n=6-8/group). Murine B16F10 melanoma cells were inoculated into the right flanks of female C57BL6/J mice. Murine RM-1 (prostate cancer) cells were grafted into the right flanks of male C57BL6/J mice. Tumor volume was measured twice weekly using a digital caliper until the end of the study (Day 21), or the humane endpoint (>2,000 mm³). When the average tumor volume reached 80-120 mm³, mice were administered a single 50 μL IT injections of 1×10⁷ TCID₅₀ of JURV or PBS. Data plotted as mean+/−SD. Paired t test with and Wilcoxen signed-rank test were performed (95% CI). Tumor volume and Kaplan-Meier survival analysis of EMT6 (a, b), RM-1 (c, d), CT26 (e, f), A20 (g, h), and B16-F10 (i, j) tumor-bearing mice. The statistical significance of differences in the survival curves between the groups was evaluated using the log-rank (Mantel-Cox) test.

FIG. 20 shows analysis of tumor-infiltrating immune cells following intratumoral injection of oncolytic JURV in murine HCC tumors. Immune profiling of PBS, JURV, anti-PD-1 therapy, JURV+ anti-PD-1 antibodies and dual flanks (abscopal effect) treated syngeneic HEPA 1-6 mice showing percentage of tumor infiltrating immune cells, including natural killer T (A, NKT), F4/80− macrophages (B), CD45+ (C), CD11b+ (D), CD11b− (E), CD3+ (F), CD8+ (G), CD8+ Granzyme+ (H), CD8+ IFNg+ (I), CD4+ CD44+ (J), CD4+ IFNg+ (K) and CD4+ KI67+ (L) cells. The Bartlett test was used to test homogeneity of variance and normality. If the p value of the Bartlett test was no less than P=0.05, ANOVA and a two-sample t test were used to compare group means. If the P value of the Bartlett test was less than 0.05, the Kruskal-Wallis and Wilcoxon rank-sum tests were used to compare group means. The figures demonstrate the potential significant difference of the gated subsets in the CD45+ population, determined by the p value.

FIG. 21 shows murine type I IFN expression in HEPA 1-6 tumor-bearing mice injected with PBS, JURV, anti-PD-1 antibodies, and JURV+Anti-PD-1 antibodies. (A) Level of antiviral cytokine (IFN-β) was measured in serum from mice treated with PBS, JURV, anti-PD-1 therapy, JURV+anti-PD-1 antibodies and dual flanks.

FIG. 22 shows proteogenomic changes in murine HCC injected with oncolytic JURV. (A) Volcano plot of mRNA expression differences for PBS vs. JURV (1×10⁷ TCID₅₀). (B) 3-D Pie slices of the numbers of differentially expressed genes (DEGs) between PBS vs. JURV. (C) Heatmap of the top 20 DEGs up or down regulated PBS vs. JURV. DEGs were determined using the limma-voom. (D) Graph showing top-scoring canonical pathways significantly enriched by treatment with PBS vs. JURV. A MixOmics supervised analysis was carried out between DEPs and DEGs based on Log 2 fold change values. Log 2 fold change of DEG×Log 2 fold change of DEP>0 with a P-value of DEG and DEP<0.05 were considered associated DEGs/DEPs.

FIG. 23 shows transcriptome and proteome analysis of anti-PD-1 therapy in murine HCC tumors. (A) Volcano plot of mRNA expression differences for PBS vs. anti-PD-1 therapy (10 mg/kg, twice a week for 3 weeks). (B) 3-D Pie slices of the numbers of differentially expressed genes (DEGs) between PBS vs. anti-PD-1 antibodies. (C) Heatmap of the top 20 DEGs up or down regulated PBS vs. anti-PD-1 antibodies. DEGs were determined using the limma-voom. (D) Graph showing top-scoring canonical pathways significantly enriched by treatment with PBS vs. anti-PD-1 antibodies. A MixOmics supervised analysis was carried out between DEPs and DEGs based on Log 2 fold change values. Log 2 fold change of DEG×Log 2 fold change of DEP>0 with a P-value of DEG and DEP<0.05 were considered associated DEGs/DEPs.

FIG. 24 shows proteogenomic response of murine HCC to combination oncolytic JURV and anti-PD-1 therapy. HEPA 1-6 tumors were harvested from mice treated with PBS or JURV+anti-PD-1 antibodies. (A) Volcano plot of mRNA expression differences for PBS vs. JURV (1×10⁸ TCID₅₀×3)+anti-PD-1 antibodies (10 mg/kg×6). (B) 3-D Pie slices of the numbers of differentially expressed genes (DEGs) between PBS vs. JURV+anti-PD-1 antibodies. (C) Heatmap of the top 20 DEGs up- or down-regulated PBS vs. JURV+anti-PD-1 antibodies. DEGs were determined using the limma-voom method. A fold-change |log FC|>1 and a false discovery rate (FDR) of 0.055 were used as a cutoff. The log FC was computed using the difference between the mean of log 2(JURV+anti-PD-1) and the mean of log 2(PBS), that is, mean of log 2(JURV+anti-PD-1)-mean of log 2(PBS). (D) Graph showing top-scoring canonical pathways significantly enriched by treatment with JURV+anti-PD-1 antibodies. A MixOmics supervised analysis was carried out between differentially expressed proteins (DEPs) and DEGs based on Log 2 fold change values. Log 2 fold change of DEG×Log 2 fold change of DEP>0 with a P-value of DEG and DEP<0.05 were considered associated DEGs/DEPs. (E) DEG/DEP expression heatmap of the 30 most up-regulated and down-regulated features DEG/DEP in JURV+anti-PD-1 antibodies vs. PBS.

FIG. 25 shows common DEGs between PBS vs. JURV vs. PBS vs. Anti-PD-I vs. PBS vs. JURV+Anti-PD-1 in murine HCC. Venn diagram showing the 35 DEGs in HEPA 1-6 tumors from the three data sets (PBS vs. Anti-PD-1, PBS vs. JURV+Anti-PD-1, PBS vs. JURV).

FIG. 26 shows deciphering the mechanisms of oncolytic JURV mediated anti-tumor activity in local and distant murine HCC. We performed transcriptomic analysis on HEPA 1-6 tumors harvested from the non-injected HEPA 1-6 tumors in the abscopal effect cohort. (A) Volcano plot of mRNA expression differences for PBS vs. dual flanks (non-injected tumors). (B) 3-D Pie slices of the numbers of differentially expressed genes (DEGs) between PBS vs. dual flanks (non-injected tumors). (C) Heatmap of the top 20 DEGs up or down regulated PBS vs. dual flanks (non-injected tumors). DEGs were determined using the limma-voom method as described in FIG. 24. (D) Graph showing top-scoring canonical pathways significantly enriched by treatment with PBS vs. dual flanks (non-injected tumors). A MixOmics supervised analysis was carried out between DEPs and DEGs based on Log 2 fold change values. Log 2 fold change of DEG×Log 2 fold change of DEP>0 with a P-value of DEG and DEP<0.05 were considered associated DEGs/DEPs.

FIG. 27 shows top canonical pathways enriched in JURV vs. Dual flanks (abscopal effect). Top immune-related canonical pathways enriched in comparison JURV vs. Dual flanks.

FIG. 28 shows oncolytic JURV induces a robust virus-mediated oncolysis dependent tumor growth delay in JURV in Hep3B Xenografts. Female NOD.Cg-Prkd^{scid}/J (Strain #:001303) mice (n=6/group) were inoculated subcutaneously with human luciferase expressing HEP3B cells. (A) Photon emission from mice with subcutaneous HEP3B tumors. When average tumor volume was over 80-120 mm³, mice were divided into two groups and received IT injections with either PBS, JURV at a dose of 1×10⁷ TCID₅₀ on days 0, 7 and 14. Photon counts were obtained on weeks 1, 2, and 3. Photon emission at the right dorsal flanks (B) and ventral (C) were significantly (P<005) reduced among JURV-treated mice than in PBS. (D) A digital caliper was used to determine tumor volume twice weekly until a humane endpoint, death, or end of study (Day 21). Data plotted as mean+/−SD. Paired t test with and Wilcoxen signed-rank test were performed (95% CI). HEP3B tumor treated with PBS or JURV was harvested, and we analyzed changes in protein expression. (E) Volcano plot of protein expression differences in HEP3B tumors treated with PBS vs. 1×10⁷ TCID₅₀ of JURV. (F) 3-D Pie slices of the numbers of differentially expressed proteins (DEPs) in HEP3B tumors injected with PBS vs. 1×10⁷ TCID₅₀ of JURV. (G) Heatmap of the top 20 DEPs up or down regulated in HEP3B tumors injected with PBS vs. 1×10⁷ TCID₅₀ of JURV. DEPs were determined using the limma-voom method as described in FIG. 22. A fold-change |log FC|≥1 and a false discovery rate (FDR) of 0.055 were used as a cutoff. The log FC was computed using the difference between the mean of log 2(JURV) and the mean of log 2(PBS), that is, mean of log 2-(JURV)−mean of log 2(PBS). (H) Graph showing top-scoring canonical pathways significantly enriched by treatment with 1×10⁷ TCID₅₀ of JURV in the HEP3B tumors.

FIG. 29 shows individual HEP3B tumor volume and survival. Individual tumor volume after treatment. Female NOD.Cg-Prkdcscid/J (Strain #:001303) mice (n=6/group) mice were inoculated subcutaneously with HEP3B cells tagged with a luciferase reporter protein. When tumor volume was between 80-120 mm³, mice were divided into two groups and received IT injections with either PBS or JURV at a dose of 1×10⁷ TCID₅₀ (Days 0,7 and 14). (A) Tumor volume and (B) body weight was recorded twice weekly until humane endpoint or end of study (Day 21). (C) Kaplan-Meier survival analysis of HEP3B tumor-bearing mice. The statistical significance of differences in the survival curves between the groups was evaluated using the log-rank (Mantel-Cox) test.

### DETAILED DESCRIPTION

Hepatocellular carcinoma (HCC) is the leading cause of cancer morbidity and mortality worlwide.¹ Most patients with HCC are diagnosed with advanced diseases and are left with limited therapeutic options. Current approaches for patients with HCC ineligible for surgery or liver transplantation include cytotoxic therapies, targeted therapies, and immune checkpoint inhibitors.²³ However, these treatments do not achieve long-term disease control, making HCC one of the cancers with the highest unmet clinical need globally.

Oncolytic viruses (OVs) are potent anti-cancer agents; they do not replicate in normal cells but preferentially amplify their genomes in tumor cells that cannot activate their cellular-based anti-viral defense mechanisms.^{6,7} Due to their multifaceted anti-cancer activities, comprising direct tumor cell-killing capabilities and immunomodulatory properties, OVs are becoming increasingly appealing in immuno-oncology.^{1,2} Among reported OVs, members of the Rhabdoviridae family^{2,3} have been intensively interrogated for their potential application as therapeutic agents for many years. The present invention is directed to a new member of the Rhabdoviridae family, Jurona virus (JURV) °, in treating HCC.

The Examples demonstrate that JURV induces a strong cytolytic effect in HCC cell lysis in vitro and animal models. Moreover, JURV elicits a systemic anti-tumor immunity resulting in tumor growth inhibition in both injected and non-injected tumors in a syngeneic HCC model. Furthermore, a combination of JURV and immune checkpoint blockade antibodies profoundly modulated the tumor microenvironment by favoring the activation of tumor-specific cytotoxic T cells. These compelling data demonstrate that the JURV provided herein may be used as a novel oncolytic viral therapy platform for HCC and possibly other cancers as well.

The present invention provides compositions, constructs, infectious particles, pharmaceutical compositions, methods of treatment, and systems related to the novel Jurona virus of the instant disclosure and its use to treat cancer.

### Compositions:

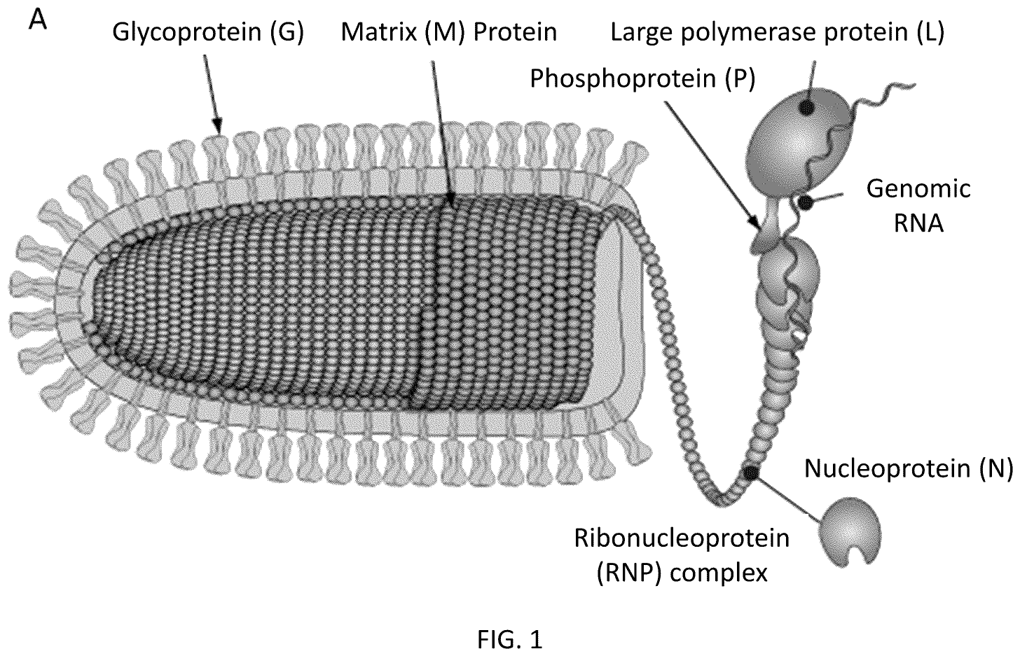

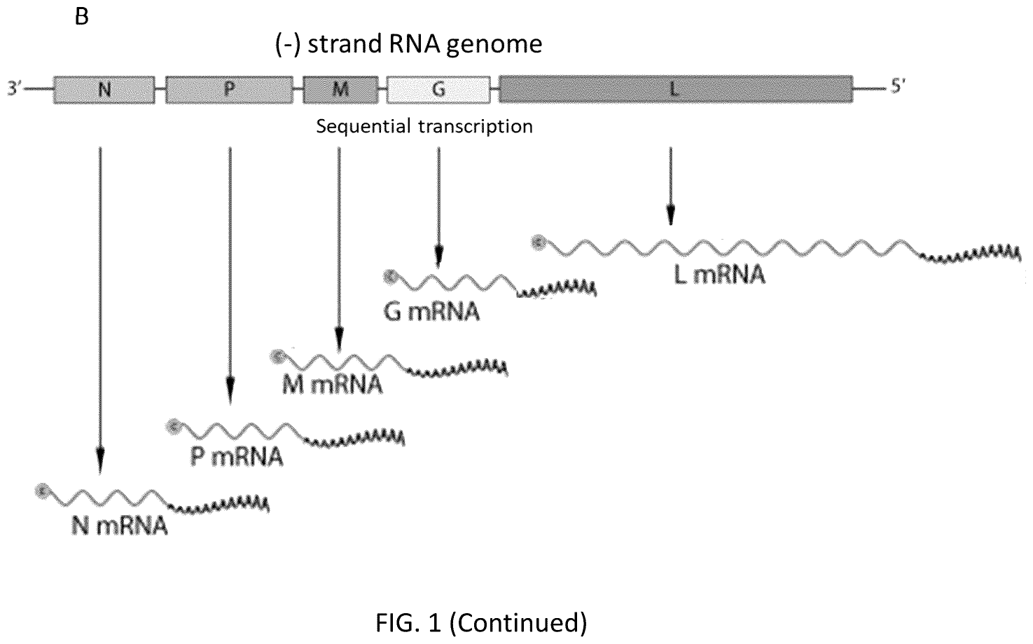

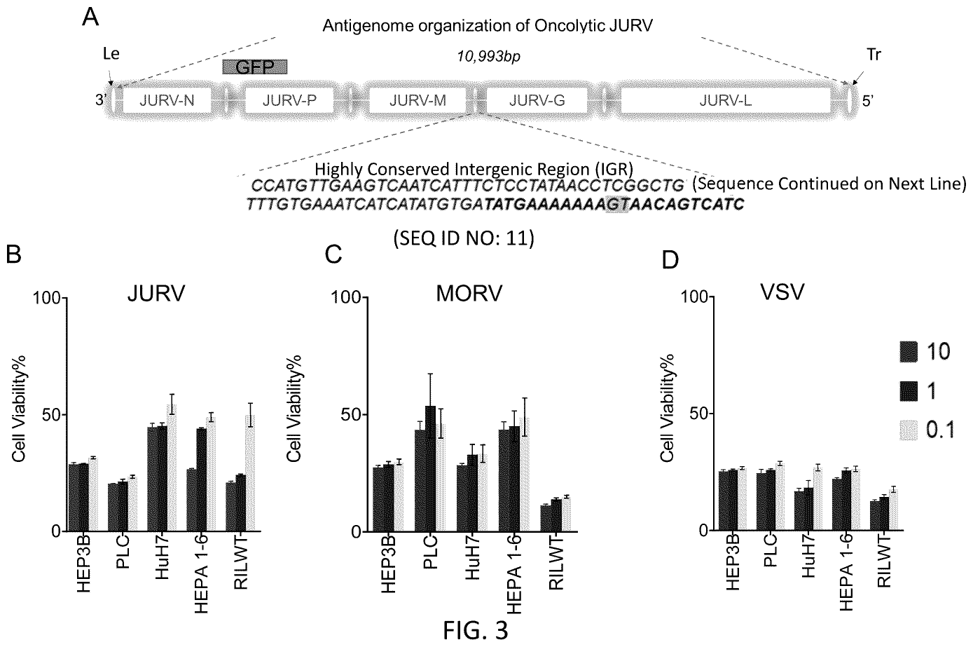

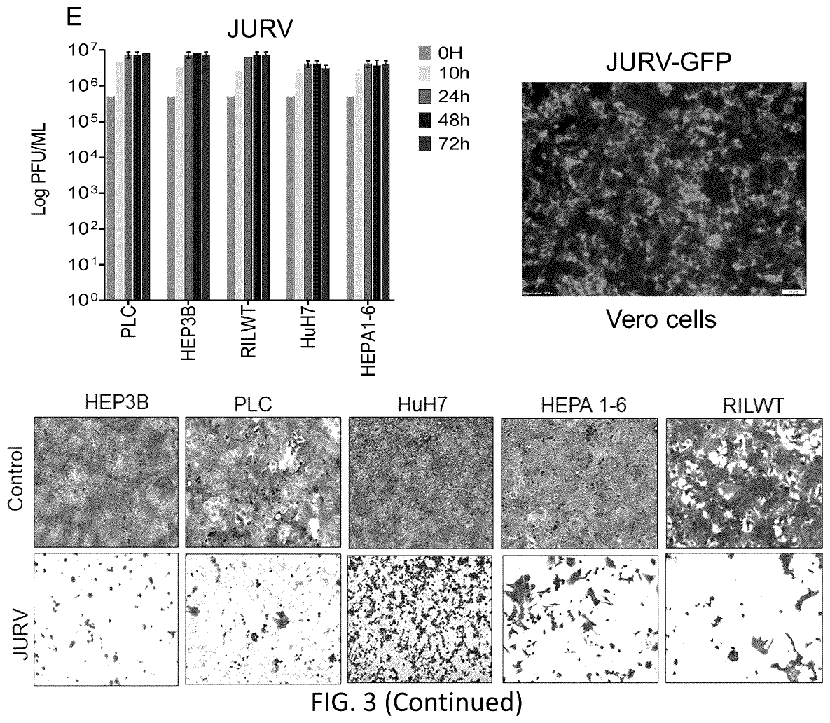

Jurona virus is (JURV) is non-pathogenic and is closely related to, yet genetically distinct from, vesicular stomatitis virus Indiana strain (FIG. 1A).^{1,2} The JURV genome is 10,993 bp linear negative sense RNA and has 5 specific Vesiculovirus genes (from the 3′ to 5′ direction in the negative sense RNA genome, and, thus, from 5′ to 3′ in a positive sense complementary RNA or complementary DNA): matrix (M), nucleoprotein (N), phosphoprotein (P), glycoprotein (G), and polymerase (L).¹ See, FIG. 3A which shows a schematic organization of the JURV genome from 3′ to 5′. Similar to other negative sense RNA viruses, e.g., vesicular stomatitis virus (VSV), influenza virus, Ebola virus, rabies virus, the viral genome encodes an RNA-dependent RNA polymerase, referred to as L protein in JURV. Thus, the L protein may directly synthesize “mRNA” from the negative sense RNA genome of the virus. Accordingly, it is to be understood that each embodiment of the disclosed compositions also contemplates a complementary sense polynucleotide, e.g., complementary sense RNA to the disclosed polynucleotides, single-stranded or double-stranded cDNA comprising the disclosed polynucleotides.

As used herein, “negative sense RNA genome” refers to the single-stranded RNA of a virus with genetic content being the antisense strand of the viral mRNA, as understood in the art. In a more general sense, “negative sense” may refer to the reverse complementary to both the positive-sense strand and the RNA transcript.

In a first aspect, constructs comprising a promoter operably linked to a polynucleotide encoding a full length Jurona virus genome. The constructs are DNA but the promoter is linked to allow for production of a negative sense viral genome when transfected into mammalian cells. Constructs include any compositions comprising DNA including but not limited to plasmids, bacterial artificial chromosomes, yeast artificial chromosomes, transposons, virus or viral vectors, recombinant chromosomal genomic DNA or any other constructs available to those skilled in the art.

To generate a functional viral particle, the constructs comprise polynucleotides encoding the M, N, P, G, and/or L proteins (on the sense strand), a leader sequence, a trailer sequence, and/or appropriate intergenic regions. The promoter may be a T7 promoter, which is responsive to an RNA polymerase from the T7 bacteriophage. As used herein, “leader sequence” refers to the region of a messenger RNA (mRNA) molecule that precedes the coding sequence of a gene, “trailer sequence” refers to the segment at the 3′ end of mRNA following the signal that terminates translation and may be untranslated and may be exclusive of the poly-A tail, and “intergenic sequence” refers to a sequence of DNA that is located between genes. The construct may comprise a polynucleotide encoding the JURV N protein having at least 90% sequence identity to SEQ ID NO: 1. The construct may comprise a polynucleotide encoding the JURV P protein having at least 90% sequence identity to SEQ ID NO: 2. The comp construct may comprise a polynucleotide encoding the JURV M protein having at least 90% sequence identity to SEQ ID NO: 3. The construct may comprise a polynucleotide encoding the JURV G protein having at least 90% sequence identity to SEQ ID NO: 4. The construct may comprise a polynucleotide encoding the JURV L protein having at least 90% sequence identity to SEQ ID NO: 5. The construct may comprise SEQ ID NOs: 1-5 or sequences having at least 90%, 92%, 94%, 95%, 97%, 98%, 99% or 100% identity thereto. As discussed above, a construct encoding a functional recombinant JURV must include a leader sequence and a trailer sequence, which may have the sequences SEQ ID NO: 6 and 7, respectively. Exemplary intergenic regions may include SEQ ID NOs: 8-11 and should be present in the polynucleotide composition in a particular order. FIG. 3A shows a schematic of the negative sense JURV genome with the polynucleotides encoding, from 3′ to 5′ the JURV N, P, M, G, and L genes. Thus, ordered from 3′ to 5′, the intergenic regions having SEQ ID NOs: 8-11 should be present in the composition with the following arrangement: SEQ ID NO: 8 between N and P, SEQ ID NO: 9 between P and M, SEQ ID NO: 10 between M and G, and SEQ ID NO: 11 between G and L. However, it should be understood that modifications may be made to the intergenic regions without significantly affecting the composition's ability to be used to generate a functional virus, and such modifications are contemplated as part of the instant disclosure. In addition, it should be understood that the order of the JURV genes may be altered, though this may result in decreased production of virus.

The construct may comprise SEQ ID NO: 12, also referred to as JURV-XN-2, which is a polynucleotide encoding the nucleoprotein (N), phosphoprotein (P), matrix protein (M), glycoprotein (G), and RNA-directed RNA polymerase L protein (L), that was codon-optimized for expression in mammalian cells by the inventors from the laboratory-adapted viral clone of JURV. In addition to the foregoing modifications from the wild-type JURV, the inventors incorporated intergenic regions from vesicular stomatitis virus into SEQ ID NO: 12.

The constructs of the instant disclosure further comprise a heterologous polynucleotide. As used herein, a “heterologous polynucleotide” refers to a polynucleotide encoding a protein (a “heterologous protein”) that is not found in Jurona virus in nature (i.e. non-native or “not natively associated”). Suitable heterologous proteins include, without limitation, reporter proteins and antigenic proteins. “Reporter protein” may refer to a protein that is expressed when certain conditions are met (e.g., when a gene is expressed). “Antigenic protein” may refer to proteins that are identified by the immune system. The reporter protein may be a fluorescent protein. As used herein, “fluorescent protein” is any protein that emits light when exposed to light. Exemplary fluorescent proteins include, without limitation, zsGreen, mRuby, mCherry, green fluorescent proteins (GFPs) and GFP variants (e.g., sfGFP), yellow fluorescent proteins (YFPs), red fluorescent proteins (RFPs), DsRed fluorescent proteins, far-red fluorescent proteins, orange fluorescent proteins (OFPs), blue fluorescent proteins (BFPs), cyan fluorescent protein (CFPs), Kindling red protein, and JRed. An “antigenic protein” is a protein that can serve as an antigen (i.e., a substance that induces an immune response). Suitable antigenic polypeptides may include, without limitation, viral antigens, bacterial antigens, fungal antigens, parasitic antigens and tumor-specific antigens. In the Examples, GFP was encoded in the recombinant viral genome as the heterologous protein and the composition includes a polynucleotide comprising SEQ ID NO: 13 and encoding a GFP tagged JURV.

The heterologous protein may be a viral antigen. Suitable viral antigens include proteins produced by viruses such as coronaviruses, alphaviruses, flaviviruses, adenoviruses, herpesviruses, poxviruses, parvoviruses, reoviruses, picornaviruses, togaviruses, orthomyxoviruses, rhabdoviruses, retroviruses, hepadnaviruses, herpesviruses, rhinoviruses, cytomegalovirus, Kaposi sarcoma virus, human papillomavirus (HPV), human immunodeficiency virus (HIV), herpes simplex virus, herpesvirus 1, herpesvirus 2, herpesvirus 6, herpesvirus 7, herpesvirus 8, hepatitis A, hepatitis B, hepatitis C, measles, mumps, parvovirus, rabies virus, rubella virus, varicella zoster virus, Ebola virus, west Nile virus, yellow fever virus, dengue virus, rotavirus, zika virus, and the like.

In another aspect of the current disclosure, constructs comprising a codon optimized polynucleotide encoding at least one Jurona virus protein selected from the group consisting of G, N, P, L, and M operably linked to a promoter for expression in mammalian cells are provided. The polynucleotide may encode the N protein and may comprise SEQ ID NO: 1. The polynucleotide may encode the P protein and may comprise SEQ ID NO: 2. The polynucleotide may encode the M protein and may comprise SEQ ID NO: 3. The polynucleotide may encode the G protein and may comprise SEQ ID NO: 4. The polynucleotide may encode the L protein and may comprise SEQ ID NO: 5.

The term “codon optimized” as used herein refers to a protein which is encoded by nucleic acid triplicates (i.e., codons) wherein the composition of said codons have been improved based on various criteria without altering the amino acid sequence of the protein. Criteria may include optimizing expression for the organism in which the protein will be expressed (e.g., expression in mammalian cells).

Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as the viral genomic RNA, messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. The term “nucleic acid” refers to individual nucleic acid residues (e.g. nucleotides and/or nucleosides). In some embodiments, “nucleic acid” refers to an oligonucleotide chain comprising three or more individual nucleotide residues.

As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.”

Nucleic acids, proteins, and/or other compositions described herein may be purified. As used herein, “purified” means separate from the majority of other compounds or entities and encompasses partially purified or substantially purified. Purity may be denoted by a weight-by-weight measure and may be determined using a variety of analytical techniques such as but not limited to mass spectrometry, HPLC, etc.

As used herein, “operably linked” refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence if it stimulates or modulates the transcription of the coding sequence in an appropriate cell. Generally, promoter transcriptional regulatory elements that are operably linked to a sequence are physically contiguous to the transcribed sequence, i.e., they are cis acting. However, some transcriptional regulatory elements, such as enhancers, need not be physically contiguous or located in close proximity to the coding sequences whose transcription they enhance.

The terms “protein”, “polypeptide”, and “peptide” are used interchangeably herein to refer to a polymer of amino acids. A “protein” typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

### Infectious Particles:

In another aspect of the current disclosure, infectious particles are provided. The infectious particles may be generated by transfecting cells with a composition such as the construct provided herein, comprising a promoter operably linked to a polynucleotide encoding a full-length antisense Jurona virus genome and allowing production of a negative sense viral genome. The cells may be mammalian cells and will generate infectious particles when the cells also produce several required Jurona virus proteins. The cells can be engineered to generate the required JURV proteins to manufacture Jurona virus infectious particles via transient transfection of constructs encoding for and capable of generating the required proteins along with the composition capable of generating the viral genome or the cells may be stably engineered to produce the viral proteins. The proteins may be only produced after after induction of an inducible promoter driving production of the viral proteins. As used herein, “infectious particles” refers to any particle capable of causing an infection of an organism or cell. Exemplary infectious particles include, but are not limited to, viral particles or virions and the like. The terms “virus,” “viral particle,” and “virion” are used interchangeably herein.

The infectious particles will contain several of the viral proteins required to generate the viral particles and the viral genome. The infectious particles may comprise JURV proteins including the N, P and/or L proteins. The infectious particles may also include the N, P, M and G proteins. Alternatively, the infectious particles may include the N, P, M, L and G proteins. The infectious particles also include a negative sense RNA genome which encodes a mRNA for each of the viral proteins as follows: the JURV N protein of SEQ ID NO: 1; the JURV P protein of SEQ ID NO: 2; the JURV M protein of SEQ ID NO: 3; the JURV G protein of SEQ ID NO: 4; and the JURV L protein of SEQ ID NO: 5. The infectious particles may comprise a negative sense RNA genome capable of encoding all of SEQ ID NOs: 1-5. The negative sense RNA genome of the infectious particles may also comprise a leader sequence and a trailer sequence. The leader and trailer sequence may be encoded for by a cDNA sequence comprising SEQ ID NOs: 6 and 7, respectively. The negative sense RNA genome of the infectious particles may comprise intergenic regions. Exemplary intergenic regions may be coded for by SEQ ID NOs: 8-11 and should be present in the polynucleotide composition in a particular order. FIG. 3A shows a schematic of the negative sense JURV genome with the polynucleotides encoding, from 3′ to 5′ the JURV N, P, M, G, and L genes. The intergenic regions, ordered from 3′ to 5′, having SEQ ID NOs: 8-11, if present in the infectious particles, should be present with the following arrangement: SEQ ID NO: 8 between N and P, SEQ ID NO: 9 between P and M, SEQ ID NO: 10 between M and G, and SEQ ID NO: 11 between G and L. The infectious particles may comprise a negative sense RNA of the polynucleotide of SEQ ID NO: 12.

The infectious particles of the instant disclosure may further comprise a heterologous polynucleotide. The heterologous polynucleotide may be a polynucleotide encoding an antigen or may encode a reporter protein. In some embodiments, the heterologous polynucleotide is GFP and the infectious particle comprises a polynucleotide of SEQ ID NO: 13. The inclusion of a reporter protein in the virus allows for detection of infected cells. An antigen can be included such that infection with the virus or infectious particles allows for expression of the antigen in a cell and induction of an immune response to the antigen delivered with the infectious particle.

The infectious particles described herein comprise a codon optimized polynucleotide encoding at least one Jurona virus protein selected from the group consisting of G, N, P, L, and M operably linked to a promoter for expression in mammalian cells. The polynucleotide encoding the G protein may comprise SEQ ID NO: 4, the polynucleotide encoding the N protein may comprise SEQ ID NO: 1, the polynucleotide encoding the P protein may comprise SEQ ID NO: 2, the polynucleotide encoding the M protein may comprise SEQ ID NO: 3, and the polynucleotide encoding the L protein may comprise SEQ ID NO: 5.

### Pharmaceutical Compositions:

In another aspect of the current disclosure, pharmaceutical compositions are provided. The pharmaceutical compositions comprise an infectious particle comprising a Jurona virus genome. The Jurona virus particle may be infectious and allow for further production of a negative sense viral genome when transfected into mammalian cells. The compositions may further comprise a pharmaceutically acceptable carrier. The infectious particles are those infectious particles described above and may comprise at least the N, P and L proteins of the Jurona virus, or alternatively, at least the N, P, M and G proteins, or alternatively the N, P, M, G and L proteins of Jurona virus.

The pharmaceutical compositions comprise infectious particles and a pharmaceutically acceptable carrier or excipient. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media.

The pharmaceutical compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

The pharmaceutical compositions may also include adjuvants to increase their immunogenicity. Suitable adjuvants include, without limitation, mineral salt adjuvants, gel-based adjuvants, carbohydrate adjuvants, cytokines, or other immunostimulatory molecules. Exemplary mineral salt adjuvants include aluminum adjuvants, salts of calcium (e.g. calcium phosphate), iron, and zirconium. Exemplary gel-based adjuvants include aluminum gel-based adjuvants and acemannan. Exemplary carbohydrate adjuvants include inulin-derived adjuvants (e.g., gamma inulin, algammulin) and polysaccharides based on glucose and mannose (e.g., glucans, dextrans, lentinans, glucomannans, galactomannans). Exemplary cytokines include IFN-γ, granulocyte-macrophage colony stimulating factor (GM-CSF), IL-2, and IL-12. Suitable adjuvants also include any FDA-approved adjuvants including, without limitation, aluminum salt (alum) and the squalene oil-in-water emulsion systems MF59 (Wadman 2005 (Novartis)) and AS03 (GlaxoSmithKline).

### Methods for Treating Cell Proliferative Diseases or Disorders:

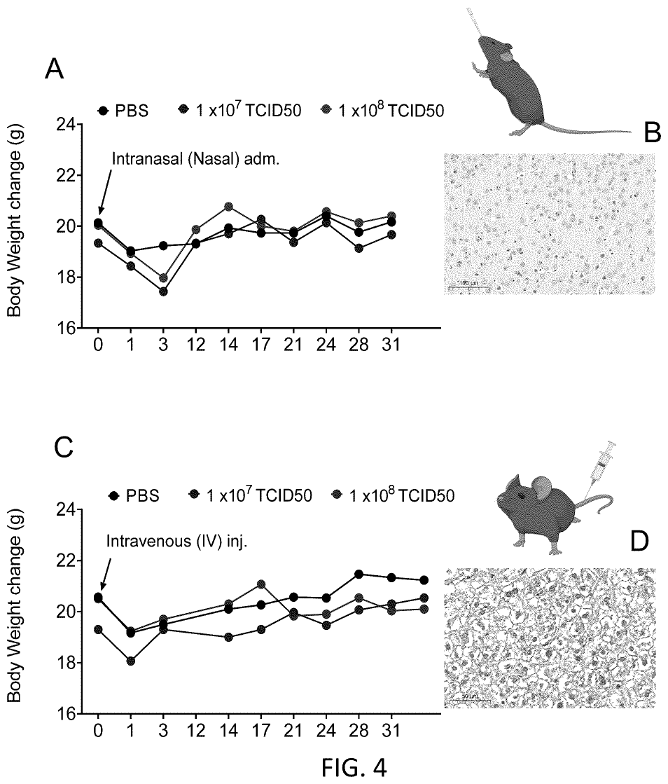

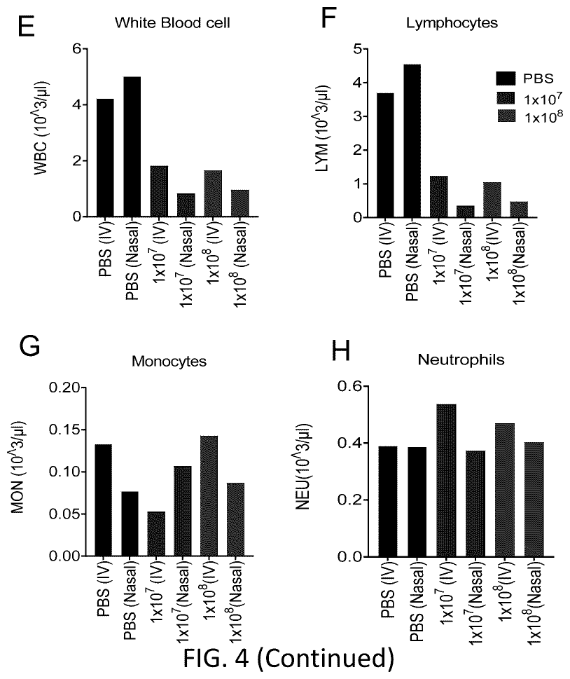

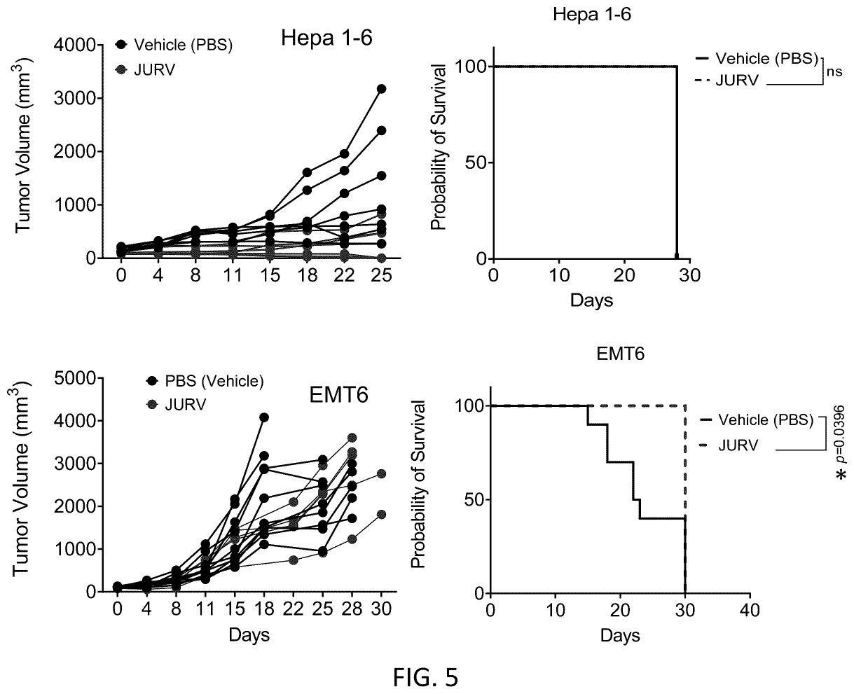

As shown in the Examples, administration of Jurona virus (JURV) to non-tumor bearing mice does not cause significant change in bodyweight when administered intranasally (FIG. 4A) or intravenously (FIG. 4B) and caused minor reductions in lymphocyte counts and little change to monocyte and neutrophil counts (FIGS. 4F-H). Therefore, JURV is non-pathogenic.

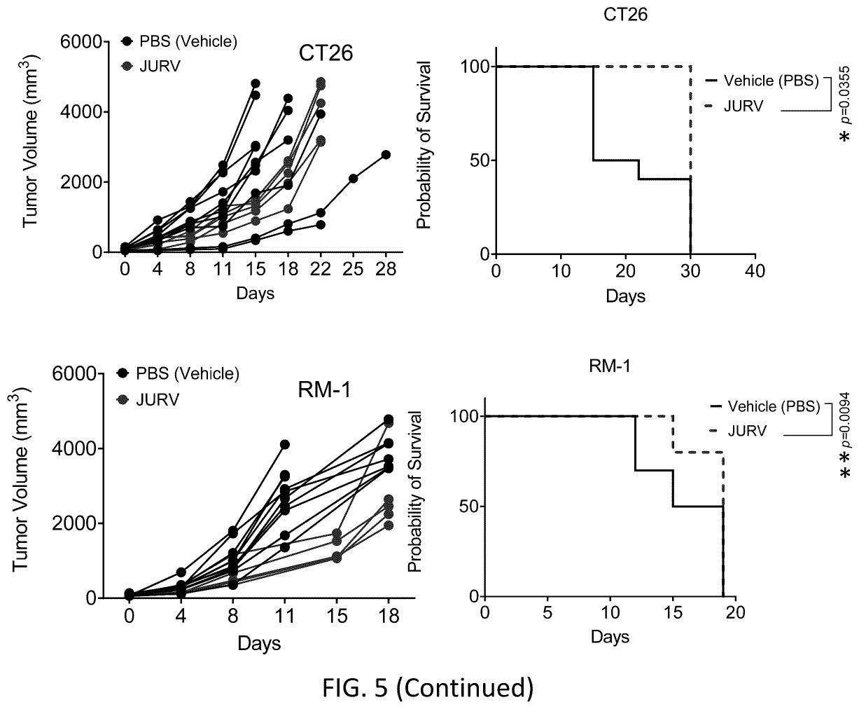

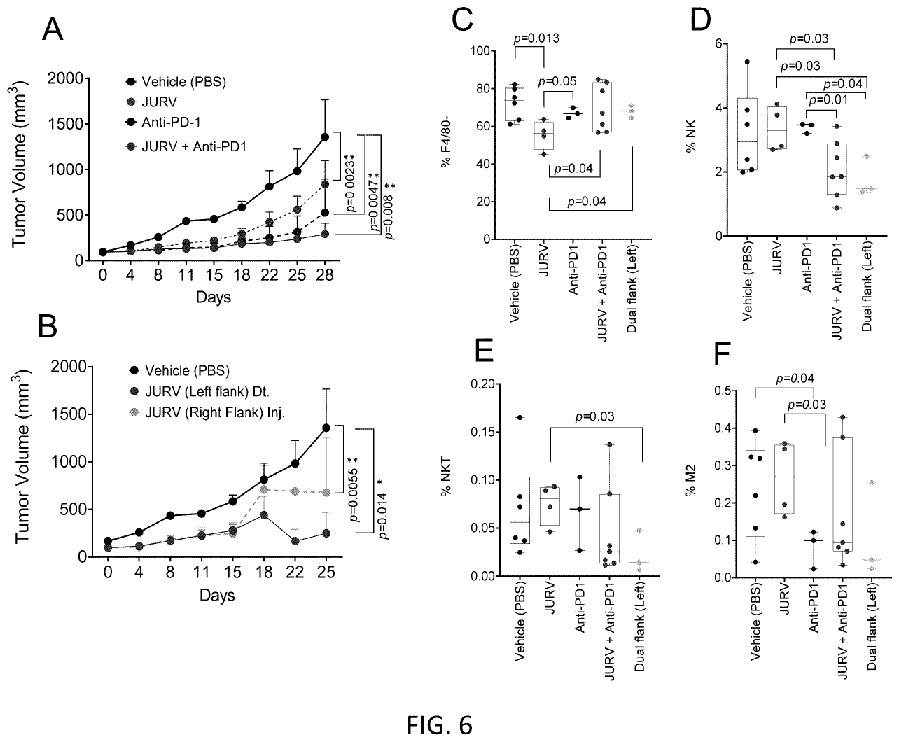

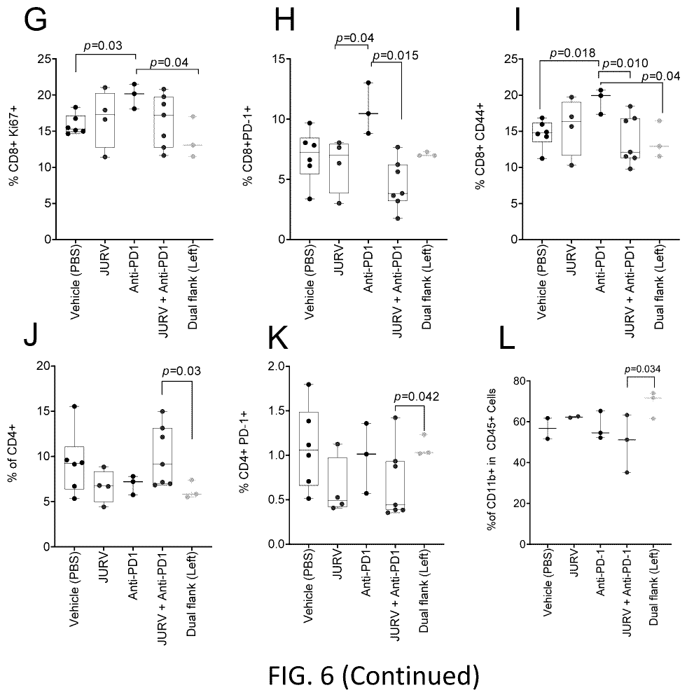

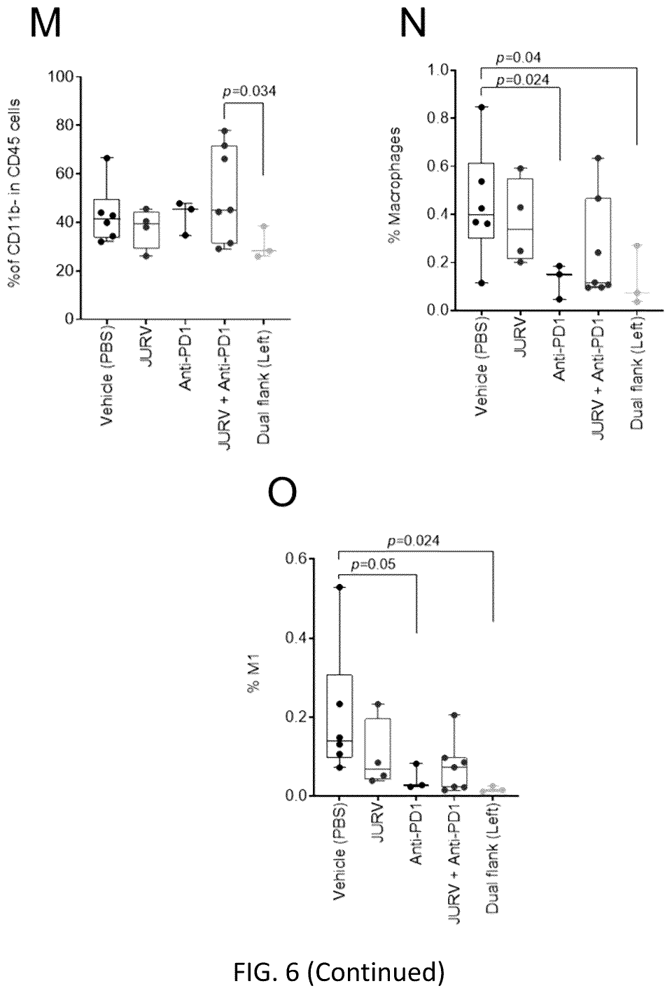

JURV was capable of reducing tumor volume and prolonging survival of tumor bearing mice in the Examples provided here. JURV administration reduced tumor volume in mice harboring Hepa 1-6 (murine hepatoma) and RM-1 (prostate gland carcinoma) tumors and prolonged survival of mice harboring EMT-6 (mammary carcinoma), CT26 (murine colorectal carcinoma), and RM-1 tumors as shown in FIG. 5. FIG. 6 further demonstrates that mice implanted with HepC3 (hepatocellular carcinoma) cells had significantly reduced tumor volume after treatment with JURV compared to control treated animals.

Accordingly, methods for treating a cell proliferative disease or disorder are provided. As used herein, “cell proliferative diseases or disorders” are any disease or disorder characterized by uncontrolled or abnormal cell growth or division. Exemplary cell proliferative diseases and disorders include, but are not limited to, cancer, carcinoma in situ, lymphoproliferative disorders, e.g., chronic lymphocytic leukemia, myeloproliferative disorders, e.g., polycythemia vera, and the like.

The methods comprise administering a pharmaceutical composition comprising an infectious particle comprising a Jurona virus genome and a pharmaceutically acceptable carrier to a subject to treat the cell proliferative disease or disorder. The cell proliferative disease or disorder can be cancer and can be selected from hepatocellular carcinoma, liver bile duct carcinoma, breast cancer, colorectal cancer, prostate cancer, and reticulum sarcoma. The breast cancer may be HER2-negative. The cancer may be local or metastatic. A “local” cancer may refer to a cancer that has not spread from its original (primary) location in the subject's body. A “metastasized” cancer may refer to a cancer that has spread from its original (primary) location in the subject's body to another (secondary) location in the subject's body. The subject may have a suppressed immune system. A suppressed immune system can be identified or determined through means known in the art and can include identifying a decline in white blood cells, monocytes, lymphocytes, neutrophils, and/or other immune cells.

The pharmaceutical compositions may be administered to a subject in combination with another agent with a similar or a different biological activity. For example, the methods may further comprise administering an immunotherapy to the subject before, at the same time as or after administration of the infectious particles comprising a Jurona virus genome or other compositions. The immunotherapy may be a checkpoint inhibitor therapy. The checkpoint inhibitor therapy may be selected from the group consisting of inhibitors of PD-1, inhibitors of PD-L1, inhibitors of CTLA-4, and inhibitors of LAG-3 (CD223). Checkpoint inhibitor therapies are known in the art. Suitable PD-1 inhibitors for use in the methods described herein are known in the art and include, but are not limited to, anti-PD-1 antibodies and anti-PD-L1 antibodies. An oncolytic adenoviral vector that encodes a monoclonal antibody specific for CTLA4, e.g., a human monoclonal antibody specific for CTLA4, (or the antibodies encoded thereby) may be used. The methods may further comprise administering an inhibitor of IFN-α to the subject before, at the same time as or after administration of the infectious particles comprising a Jurona virus genome or other composition. The methods may further comprise administering a receptor tyrosine kinase inhibitor (e.g., pazopanib) to the subject before, at the same time as or after administration of the infectious particles comprising a Jurona virus genome or other composition.

The methods comprise administering a therapeutically effective amount of the pharmaceutical composition including the Jurona virus infectious particle to the subject. As used herein, the term “therapeutically effective amount” refers to an amount of viral particle or pharmaceutical formulation that is sufficient to alleviate one or more sign or symptom of the cell proliferative disease or disorder in a subject. Exemplary signs or symptoms of a cell proliferative diseases that may be “treated” or “alleviated” by the disclosed methods include, but are not limited to, reduction in tumor volume, remission of disease, cured disease, reduction in tumor number, weight gain, increased appetite, etc. In addition, for each type of cell proliferative disease being treated by the disclosed methods, there are disease specific outcomes that represent effective treatment. For example, in the case of hepatocellular carcinoma, subjects being treated by the instant methods may experience increase in appetite, loss of pain or feeling of fullness under the ribs on the right side of the body, reduction in nausea or vomiting, and remission of jaundice, etc. Other disease specific signs and symptoms which may be treated or alleviated by the instant methods are well known in the art.

As used herein, the terms “administering”, and “administration” refer to any method of providing a pharmaceutical preparation to a subject. Suitable routes of administration include, without limitation, intramuscular, intradermal, intranasal, oral, topical, parenteral, intravenous, subcutaneous, intrathecal, transcutaneous, nasopharyngeal, intratumoral, and transmucosal routes. The pharmaceutical compositions may be administered intranasally, intramuscularly, or intratumorally. The pharmaceutical compositions can be administered as a single dose or in multiple doses. For example, the pharmaceutical compositions may be administered two or more times separated by 4 hours, 6 hours, 8 hours, 12 hours, a day, two days, three days, four days, one week, two weeks, or by three or more weeks. For instance, in the Examples, the viral particles were administered intratumorally once a week for three weeks. The does for each mouse at each administration was 1×10⁷ TCID⁵⁰. Those of skill in the art will be able to calculate a dose for administration depending on the tumor and subject being treated. Thus, in some embodiments, the viral particle is administered to the subject at least twice.

The “subject” to which the present methods are applied may any vertebrate. Suitable vertebrates include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

### Cells:

In another aspect of the current disclosure, cells are provided. The cells comprise a promoter operably linked to a polynucleotide encoding a Jurona virus genome and allow production of a negative sense viral genome. The cells may further comprise constructs or be genetically engineered for production of the viral proteins needed to allow for assembly of infectious particles. The cells may be engineered to produce the viral proteins required for assembly of infectious particles after induction. Those of skill in the art will appreciate that these cells may be engineered by stable integration of constructs including an inducible promoter operably connected to polynucleotides encoding the necessary Jurona virus proteins selected from the group consisting of N, P, M, G and L proteins of Jurona virus provided herein as SEQ ID NO: 1-5, respectively. The cells may alternatively be transfected with one or more plasmid allowing for production of the necessary proteins.

In some embodiments, the cells comprise SEQ ID NO: 12, also referred to as JURV-XN-2, which is a polynucleotide encoding the negative sense RNA genome of the codon optimized, mammalian cell adapted Jurona virus provided herein. SEQ ID NO: 12 encodes the negative sense RNA which can be transcribed by the viral RNA polymerase to produce the nucleoprotein (N), phosphoprotein (P), matrix protein (M), glycoprotein (G), and RNA-directed RNA polymerase L protein (L), that have been codon-optimized for expression in mammalian cells from the laboratory-adapted viral clone of JURV. In addition to the foregoing modifications from the wild-type JURV, the inventor incorporated intergenic regions from vesicular stomatitis virus into SEQ ID NO: 12. The compositions may further comprise a polynucleotide encoding a heterologous protein, and the heterologous protein may be GFP and the composition may comprise the sequence SEQ ID NO: 13. Also encompassed are sequences having at least 95%, 96%, 97%, 98% and 99% identity to SEQ ID NO: 12 or SEQ ID NO: 13.

### Methods for Producing Viral Particles:

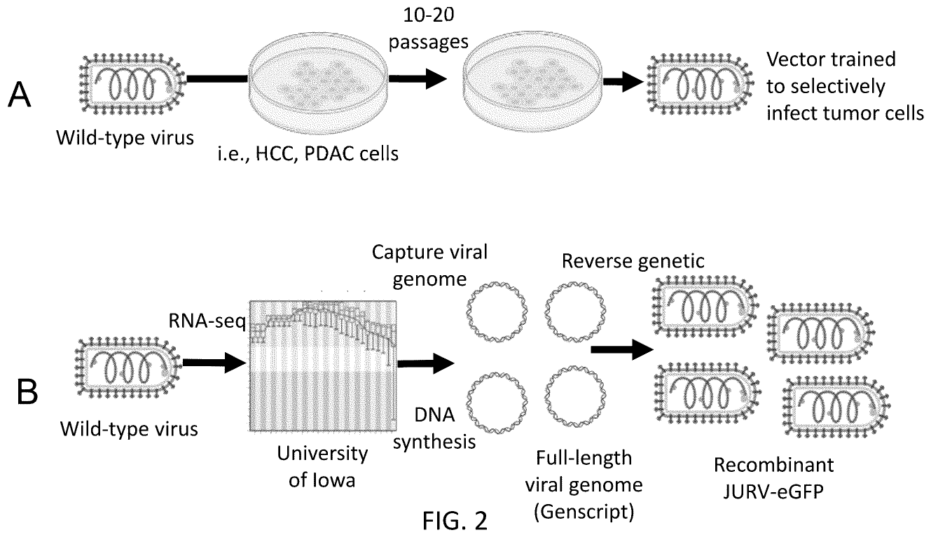

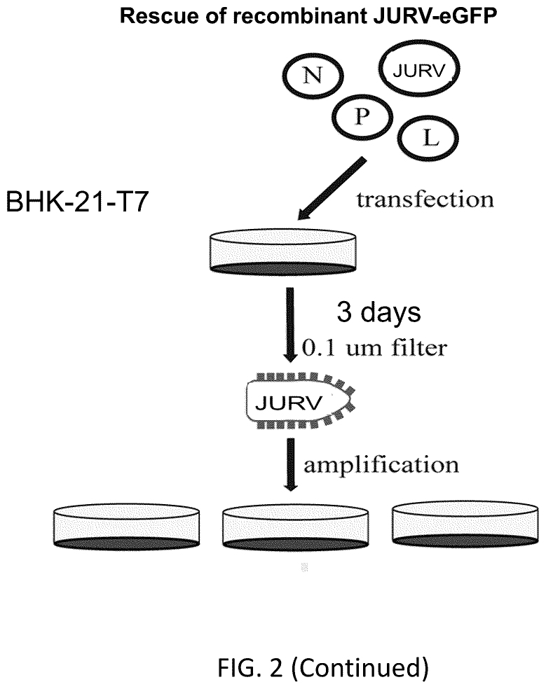

The present invention also provides methods for producing the viral particles described herein. The inventor discovered that production of JURV in mammalian cell culture could be performed by transfecting cells which express an exogenous polymerase with a polynucleotide encoding the full-length JURV genome, i.e., encoding M, P, L, G, and N, proteins, leader and trailer sequences, and intergenic regions, and comprises additional promoters operably linked to one or more polynucleotides encoding JURV proteins. See, for example, FIG. 2. Thus, when the polynucleotide is transfected into the cells with an exogenous polymerase, e.g., T7 polymerase, transcribes critical JURV transcripts that are translated to functional proteins which are required for the effective assembly of viral particles. The polynucleotides encoding the complete JURV genome, and the additional promoters operably linked to one or more polynucleotides encoding JURV proteins required for JURV assembly and functional JURV viral particle formation may be encoded on a single polynucleotide. In other embodiments, the complete JURV genome may be encoded on one polynucleotide and the polynucleotides encoding one or more JURV proteins required for viral assembly may be encoded on one or more separate polynucleotide molecules. Critically, for viral assembly to take place in the cell, the complete JURV genome must be present along with one or more JURV proteins, selected from M, P, L, G, and N proteins, which are encoded by one or more polynucleotides operably linked to promoters.

Therefore, in some embodiments, the methods comprise introducing a composition comprising a promoter operably linked to a polynucleotide encoding a Jurona virus genome and allowing production of a negative sense viral genome when transfected into mammalian cells; allowing the cell to express one or more Jurona virus proteins selected from the group consisting of G, M, N, L and P; incubating the cells for a sufficient time to generate recombinant Jurona virus; and harvesting virus produced by the cells. The one or more JURV proteins required for infectious particle production may comprise the N, P, and L proteins. The one or more Jurona virus proteins may be encoded by one or more polynucleotides comprising SEQ ID NOs: 1, 2, or 5. The promoter may be T7 promoter and the cell may comprise T7 polymerase.

The polynucleotide encoding the Jurona virus genome may additionally comprise a heterologous polynucleotide encoding a protein not natively associated with a Jurona virus. The heterologous polynucleotide may encode an antigen or a reporter protein, as noted above.

As used herein, the terms “transfecting” and “transfection” refer to a process of artificially introducing nucleic acids (DNA or RNA) into cells. Transfection may be performed under natural or artificial conditions. Suitable transfection methods include, without limitation, lipofection, bacteriophage or viral infection, electroporation, heat shock, microinjection, and particle bombardment.

As used herein, the terms “infecting” and “infection” refer to a process of introducing a virus into a cell. Cells may be infected with a virus by simply contacting the cell with viral particles.

The cell lines used in the present methods are eukaryotic cell lines. Suitable eukaryotic cells include, without limitation, mammalian cells or chicken cells. The cell may be a cell in culture. Suitable mammalian cells include, without limitation, a BHK-21 cell, a MDCK cell, an A549 cell, a CHO cell, a HEK293 cell, a HEK293T cell, a HeLa cell, an NS0 cell, an Sp2/0 cell, a COS cell, a BK cell, an NIH3T3 cell, an FRhL-2 cell, an MRC-5 cell, a WI-38 cell, a CEF cell, a CEK cell, a DF-1 cell, or a Vero cell. In some embodiments, the cell is a BHK-21 cell and may express T7 polymerase.

The methods for producing viral particles may further include additional steps that involve harvesting the Jurona virus from the cell. In embodiments that utilize cultured cells, the methods may further comprise harvesting the supernatant of the culture by, for example, centrifugation or pipetting. The Jurona virus harvested from the cells may be further isolated or purified from the cells and media via methods known to those of skill in the art such as density gradient centrifugation.

### Systems for Generating Recombinant Jurona Virus:

Systems for generating a recombinant Jurona virus are also provided. The systems comprise: a) one or more vectors comprising polynucleotides encoding at least three Jurona virus proteins selected from the group consisting of G, N, P, L, and M each operably linked to a promoter to allow for expression of the at least three proteins in a mammalian cell; b) a vector comprising a polynucleotide including a negative sense Jurona virus genome operably linked to a promoter to allow production of the negative sense Jurona virus genome in a mammalian cell. Thus, the disclosed systems allow for the efficient production of recombinant JURV and may further comprise: (c) mammalian cells capable of expressing the Jurona virus proteins of (a) and the negative sense Jurona virus genome of step (b) to produce the recombinant Jurona virus. The cells of the system may comprise T7 RNA polymerase. The cells may be BHK-21 cells. The one or more vectors comprise polynucleotides encoding Jurona virus N, P, and L proteins operably linked to a promoter. The polynucleotides may comprise any one of SEQ ID NOs: 1-5. The one or more vectors may comprise a codon optimized polynucleotide encoding at least one Jurona virus protein selected from the group consisting of G, N, P, L, and M operably linked to a promoter for expression in mammalian cells. The polynucleotide may encode the N protein of SEQ ID NO: 1. The polynucleotide may encode the P protein of SEQ ID NO: 2. The polynucleotide may encode the M protein of SEQ ID NO: 3. The polynucleotide may encode the G protein of SEQ ID NO: 4. The polynucleotide may encode the L protein of SEQ ID NO: 5.

The one or more vectors comprises a polynucleotide comprising a promoter operably linked to a polynucleotide encoding a Jurona virus genome allowing production of a negative sense viral genome when transfected into mammalian cells.

### Kits for Treating Cell Proliferative Diseases or Disorders:

Kits are provided. The kits comprise a pharmaceutical composition comprising an infectious particle comprising a Jurona virus genome and optionally a pharmaceutically acceptable carrier. The kits may further comprise an immunotherapy. The immunotherapy may be a checkpoint inhibitor therapy. The checkpoint inhibitor therapy may be selected from the group consisting of inhibitors of PD-1, inhibitors of PD-L1, inhibitors of CTLA-4, and inhibitors of LAG-3 (CD223). Checkpoint inhibitor therapies are known in the art. Suitable PD-1 inhibitors for use in the methods described herein are known in the art and include, but are not limited to, anti-PD-1 antibodies and anti-PD-L1 antibodies. In some embodiments an oncolytic adenoviral vector that encodes a monoclonal antibody specific for CTLA4, e.g., a human monoclonal antibody specific for CTLA4, (or the antibodies encoded thereby) may be used. The kits may further comprise an inhibitor of IFN-α. The kits may further comprise a receptor tyrosine kinase inhibitor (e.g., pazopanib).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The following example demonstrates the generation of recombinant Jurona virus and use thereof in treating cell proliferative diseases.

### Example 1: Characterization of Jurona Virus (JURV) and its Cytopathic Effect in Tumor Cells

Oncolytic viruses can provide multimodal anti-tumor activity from selective tumor cell killing to promoting systemic anti-tumor immunity, making them a formidable foe against cancer. Among these, several members of the Rhabdoviridae family are particularly attractive as oncolytic agents due to their natural tumor selectivity and non-pathogenicity in humans. In this example, we characterize JURV and demonstrate its cytopathic effect in tumor cells.

### Results

### Characterization and Generation of Oncolytic Recombinant Jurona Virus (JURV)

Jurona virus (JURV) is non-pathogenic and is closely related to, yet genetically distinct, from vesicular stomatitis virus Indiana strain (FIG. 1).^{1,2} Of the Order Mononegavirales, Family Rhabdovirdae, and Genus Vesiculovirus (which comprises about 5000 members), JURV is enveloped, bullet shaped, and approximately 180 nm long and 75 nm wide (FIG. 1A). Vesicular stomatitis virus (prototype vesiculovirus) has a negative-stranded RNA linear genome, about 11 kb in size, and encodes for 5 proteins (FIG. 1). JURV were trained to selectively infect tumor cells using the method illustrated in FIG. 2 which shows a schematic of the method used to derive a laboratory adapted Jurona virus clone (FIG. 2A) and the process of developing a reverse genetics-based method of generating recombinant Jurona virus (FIG. 2B). The JURV genome is 10,993 bp linear RNA and has 5 specific Vesiculovirus genes (from the 3′ to 5′ direction): nucleoprotein (JURV-N), phosphoprotein (JURV-P), matrix (JURV-M), glycoprotein (JURV-G), and polymerase (JURV-L) (FIG. 3A).¹ Between JURV-M and JURV-G regions is a highly conserved intergenic region comprising SEQ ID NO: 11.

### JURV Induced Robust Cytopathic Effect (CPE) in Tumor Cells

To evaluate the cytotoxicity of JURV in vitro, we performed CPE assays on 3 human HCC cells (Hep3B, PLC, Huh7) and 2 mouse HCC cells (Hepa 1-6, RILWT) (FIG. 3B-E). We infected monolayers of human and murine liver cancer cells with JURV, VSV and MORV at MOIs of 10, 1, and 0.1; cell viability was measured 72 hours post-infection. JURV, MORV and VSV were able to lyse cancer cells and displayed relatively similar lytic activities in the tested cancer cell lines (FIG. 3B). These results indicate that JURV can selectively infect and lyse tumor cells in vitro, while leaving normal cells unaffected, although the infectivity of JURV was similar to that of VSV and MORV and could be grown at comparably high titers and infect Vero cells (FIG. 3E).

High-Dose Intranasal Administration of JURV is not Associated with Neurotoxicity or Hepatotoxicity

To determine whether JURV is causally associated with brain damage and neurotoxicity in animal models, immunocompetent mice were subjected to 2 intranasal administration (IN) and intravenously (IV) of JURV (low dose: 1×10⁷ TCID₅₀; high-dose: 1×10⁸ TCID₅₀) (FIG. 4). The control group of animals was treated with phosphate-buffered saline (PBS). Three mice per group were sacrificed 3 days post-infection to assess short-term toxicity; blood, brain, liver, and spleen tissues were collected for further analysis. The remaining animals were monitored for 45 days by a certified veterinarian for signs of toxicity. We found that low and high IN and IV doses of JURV were well tolerated and managed in all groups (FIG. 4A-D). However, it is important to note that in this study, we used an attenuated laboratory adapted strain of JURV (serial passages) which is known to be significantly attenuated, compared to wild-type virus. Decreases in body weight were not statistically significant 3 days after treatment in the JURV groups; from 7 days after IN and IV administration until the end of the study, body weights consistently increased. Consistent with the minimal effects on body weight, a thorough pathology review of brain and liver sections of mice treated with the highest IN and IV doses of viruses (1×10⁸ TCID₅₀) showed that JURV did not cause overt signs of toxicity (FIG. 4B, C). Analysis of complete blood counts 3 days after treatment with JURV revealed decreases in the numbers of white blood cells, lymphocytes, granulocytes, and platelets (FIG. 4E-H). In addition to virus-mediated hematological changes, these changes in blood cell counts might have been caused by hemolysis from the terminal cardiac puncture, which is known to interfere with blood counts and other blood parameters.

### JURV Induced Tumor Regression and Improved Survival in Mouse HCC (Hepa 1-6), Breast (EMT6), Colon (CT26) and Prostate (RAM-1) Cancers

To assess the anti-tumor efficacy of JURV across multiple murine cancer types, Hepa 1-6 (HCC), EMT6 (breast), CT26 (colon) and RM-1 (prostate) cell lines were subcutaneously implanted into female C57BL/6J mice (n=7/group) (Jackson Laboratories) (FIG. 5). After tumors reached a treatable size (80-120 mm³), three intratumoral (IT) injections (once weekly) of JURV were administered at 1×10⁷ TCID₅₀ units. Tumor volume and survival were recorded. In the Hepa 1-6 (p=0.006) model, JURV induced significant tumor growth inhibition. However, in the CT26 (p=0.0355), EMT6 (p=0.0396), RM-1 (p=0.0094) models, JURV mediated a prominent gain in survival compared to PBS (FIG. 5). We did not find adverse events or virus-related toxicities. The mechanism behind the heterogeneity in antitumor efficacy of JURV in animal models and human liver cancer is worth further investigation.

### Anti-Tumor Activity of JURV Against Localized and Untreated Distant Mouse HCC

Oncolytic vesiculoviruses exert their antitumor actions by inducing direct cytotoxicity of tumor cells and stimulating host antitumor immune responses (FIG. 6). Thus, to understand whether injection of JURV leads to immune responses against tumor cells, we subcutaneously implanted Hepa 1-6 cells into C57BL/6J mice (n=7/group). Once tumors reached 80-120 mm³, mice received three IT injections of JURV (1×10⁷ TCID₅₀) or murine anti-PD-1 (10 mg/kg daily for 14 days) or JURV+anti-PD-1. We assessed changes in tumor volume and immune cell profiles in the Hepa 1-6 tumors at the end of study (EOS). There was a significant decrease in tumor volumes in the JURV (p=0.008) treated group, anti-PD-1 (p=0.0047) and JURV+anti-PD-1 (p=0.0023) groups compared to PBS. Interestingly, the tumor growth arrest induce by JURV was similar of that of anti-PD1 and the combination JURV+anti-PD-1 was not better than JURV alone (FIG. 4.A). Additionally, we observed tumor regression in both JURV treated and non-treated tumors in mice bearing Hepa 1-6 in the right and left flanks (FIG. 6B). For the flow cytometry analysis, compared to the JURV+anti-PD1 treated group, the CD4+PD-1+ T cells in the dual flank (JURV) group were significantly increased (FIG. 6K). The CD8+CD44+ T cells were significantly increased in the anti-PD1 group as compared to the PBS groups, the JURV+anti-PD1, and the dual flank (JURV) (FIG. 6I). The proliferation of the CD8+(CD8+Ki67+) T cells was significantly increased in the anti-PD-1 group as compared to the PBS groups and the dual flank (JURV) (FIG. 6G). The CD8+PD1+ T cells were significantly increased in the anti-PD1 group than in the JURV and the JURV+anti-PD1; additionally, the CD8+PD1+ T cells were significantly increased in the dual flank (JURV) group as compared to the JURVV+Anti-PD1 group (FIG. 6H). The NK cells in the JURV and the anti-PD1 were increased dramatically as compared to the groups of JURV+anti-PD1 and the dual flank (JURV) (FIG. 6D). Compared to the PBS control group, the M1 macrophages in the anti-PD1 and the dual flank (JURV) groups were significantly decreased; compared to the PBS groups and the JURVV, the M2 macrophages in the Anti-PD1 group were significantly reduced (FIG. 6C, 6F, 6N, 6O). These results suggest that IT administration of JURV is associated with an effective and robust antitumor immune response, mainly via CD8⁺ T-cell-mediated cytotoxicity.

### Low Dose of JURV Efficiently Reduced Tumor Burden in HCC Xenografts

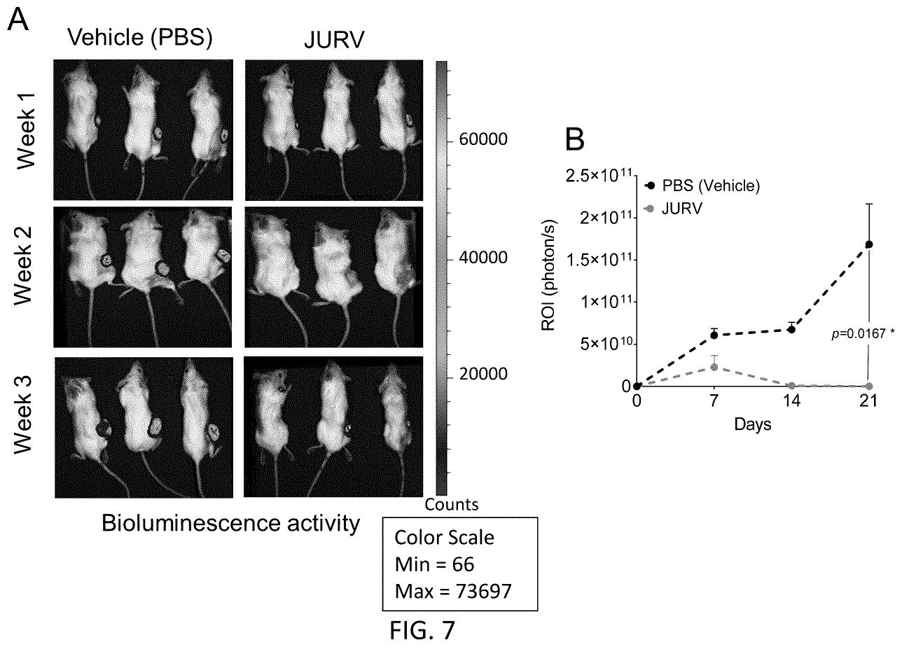

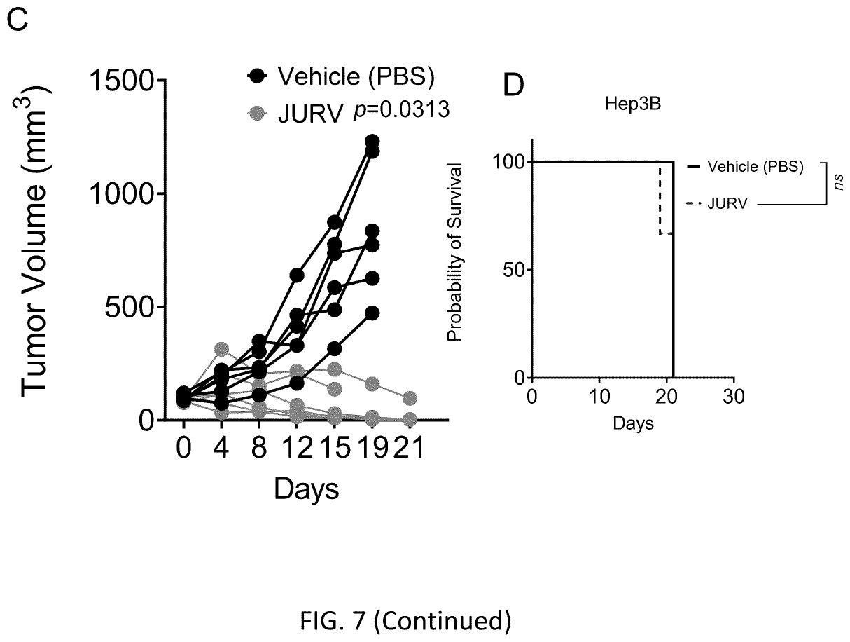

To evaluate the cancer-killing property of JURV particles in vivo, we subcutaneously implanted bioluminescent human HCC cells (Hep3B) into the right flanks of the immune-deficient mice (FIG. 7). Once tumors reached 80-120 mm³, three IT doses of PBS, or JURV (1×10⁷ TCID₅₀) were administered to mice (FIG. 7A). Bioluminescent imaging at days 7, 14 and 21 correlated with tumor growth inhibition in Hep3B mice (FIG. 7B-D). Accordingly, measurement of tumor size in the JURV vs PBS groups showed a significant tumor regression in the JURV cohort compared to PBS.

### Materials and Methods

### Cell Lines

This study used a panel of 3 human hepatocellular carcinoma (HCC) cell lines (Hep3B, PLC, Huh7) and 2 murine HCC cell line (Hepa 1-6, RILWT). All cell lines were cultured at 37° C. with 5% CO₂ in medium supplemented with L-glutamine and antibiotics (100 μg ml⁻¹ penicillin and 100 μg ml⁻¹ streptomycin). All HCC cells were and maintained in Dulbecco's modified Eagle's medium (DMEM) with 10% fetal bovine serum (FBS). BHK-21 and Vero cells were obtained from the American Type Culture Collection (ATCC; Manassas, VA). Hep3B, PLC and Huh7 and Hepa 1-6 were purchased from the ATCC. RILWT (gift from Dr. Dan Duda) is a clone derived from the RIL-175 cell line was grown in DMEM with 10% FBS.

### Oncolytic Viruses

We obtained JURV from the University of Texas Medical Branch (UTMB) World Reference Center for Emerging Viruses and Arboviruses (WRCEVA). A laboratory-adapted viral clone of JURV was generated with sequential plaque purifications on Vero cells (ATCC). RNA-sequencing was applied to confirm the full-length JURV genome (Iowa State University Veterinary Laboratory). The full-length JURV genome (10,993 nucleotides) comprised of genes encoding the nucleoprotein (JURV-N), phosphoprotein (JURV-P), matrix protein (JURV-M), glycoprotein (JURV-G), and RNA-directed RNA polymerase L protein (JURV-L), was codon-optimized for expression in mammalian cells and synthesized (Genscript) from the laboratory-adapted viral clone of JURV and was subcloned into plasmid (JURV-XN2). However, we inserted between the 5 genes of JURV intergenic regions derived from VSV to increase translation of viral proteins. JURV-XN2 and JURV-eGFP (carrying a GFP gene between the G and L genes), along with helper plasmids (JURV-P, JURV-N, and JURV-L), were used to generate recombinant JURV and JURV-GFP as previously described.⁵⁰ These plasmids were used to express the antigenomic-sense RNA of JURV under the bacteriophage T7 promoter to generate recombinant JURV; however, in the in vivo study, we used the laboratory strain of JURV rather than recombinant JURV. JURV and JURV-GFP was rescued on BHK-21 cells from a plasmid (JURV-XN2 and JURV-eGFP). All viruses were rescued with a vaccinia rescue system and were propagated and titrated on BHK-21 cells, as previously described.^{3,4} We used several concentrations of JURV-XN2 and JURV-eGFP and only the following concentration yielded infectious clone: JURV-XN2 or JURV-eGFP (5 μg), JURV-N(5 μg), JURV-P (3 μg), JURV-M (2 μg), JURV-G (0.1 μg) and JURV-L (1 μg). Sucrose density gradient centrifugation was used to obtain purified JURV particles before in vitro and in vivo studies.

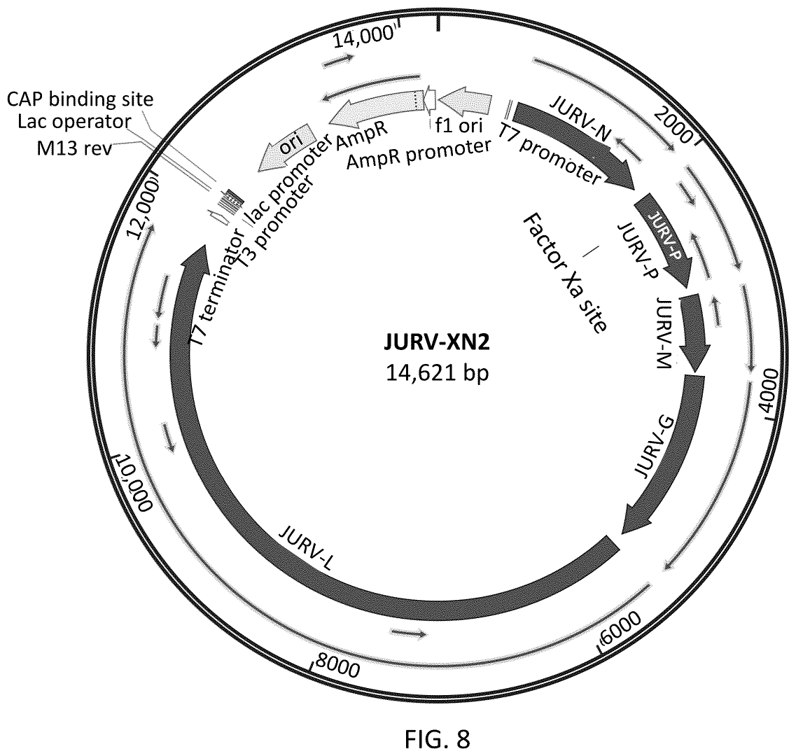

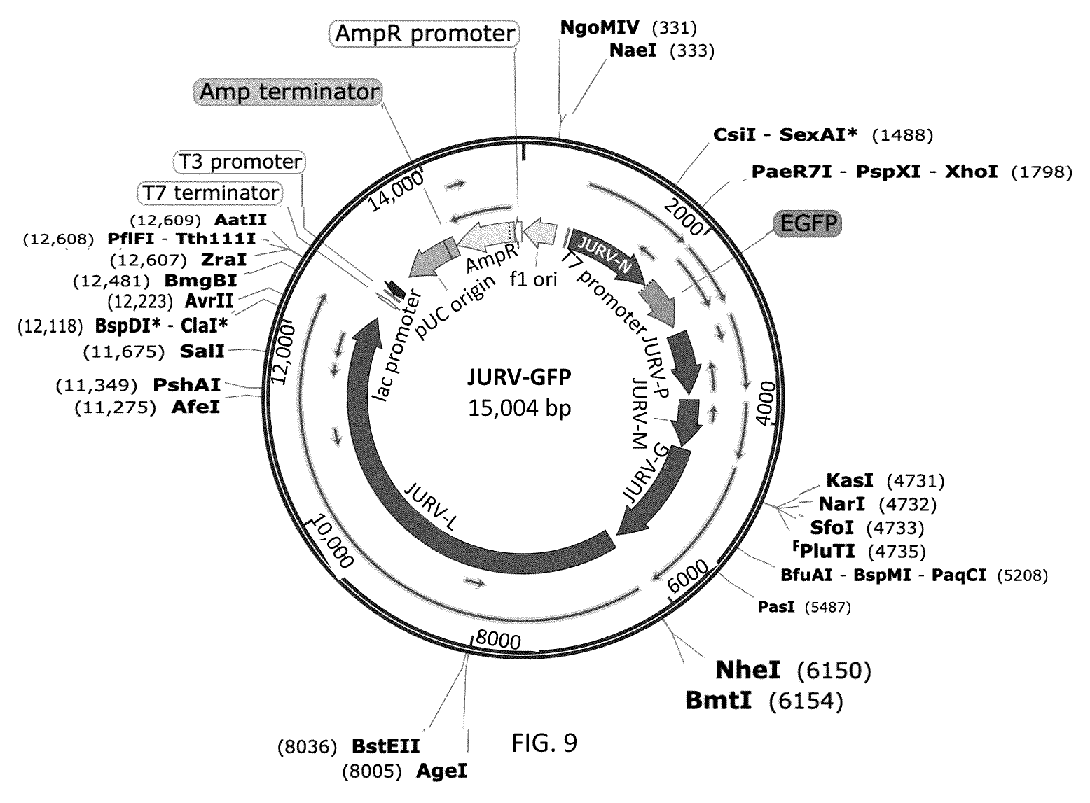

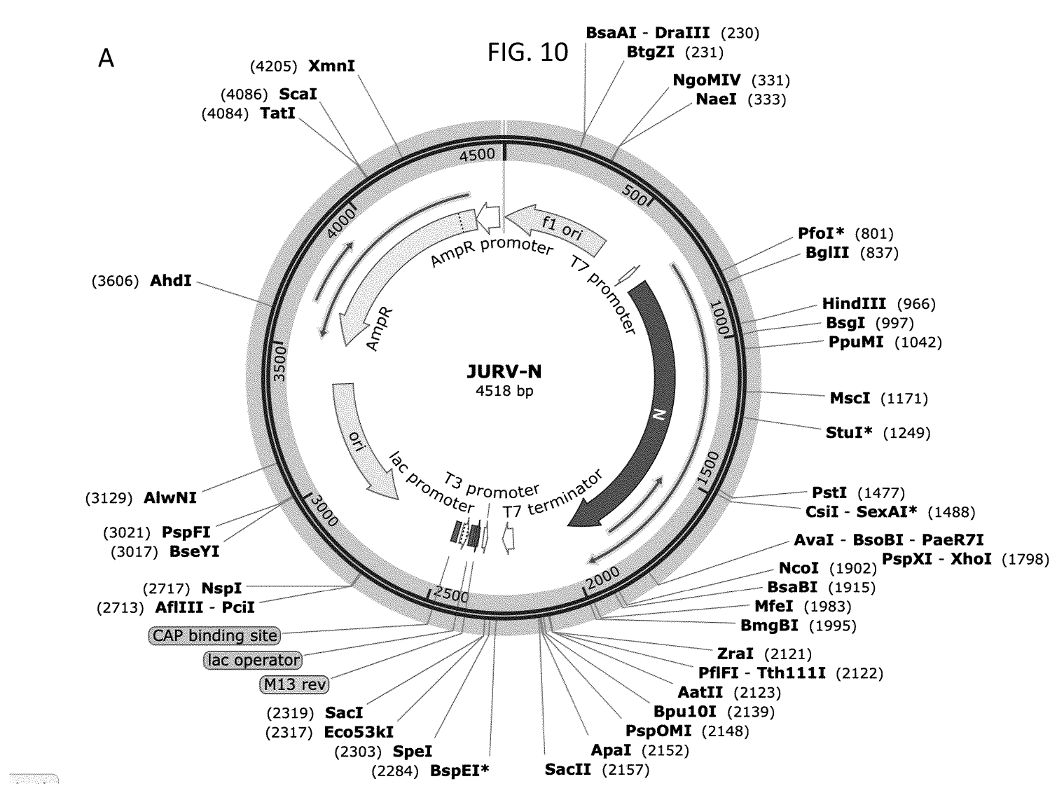

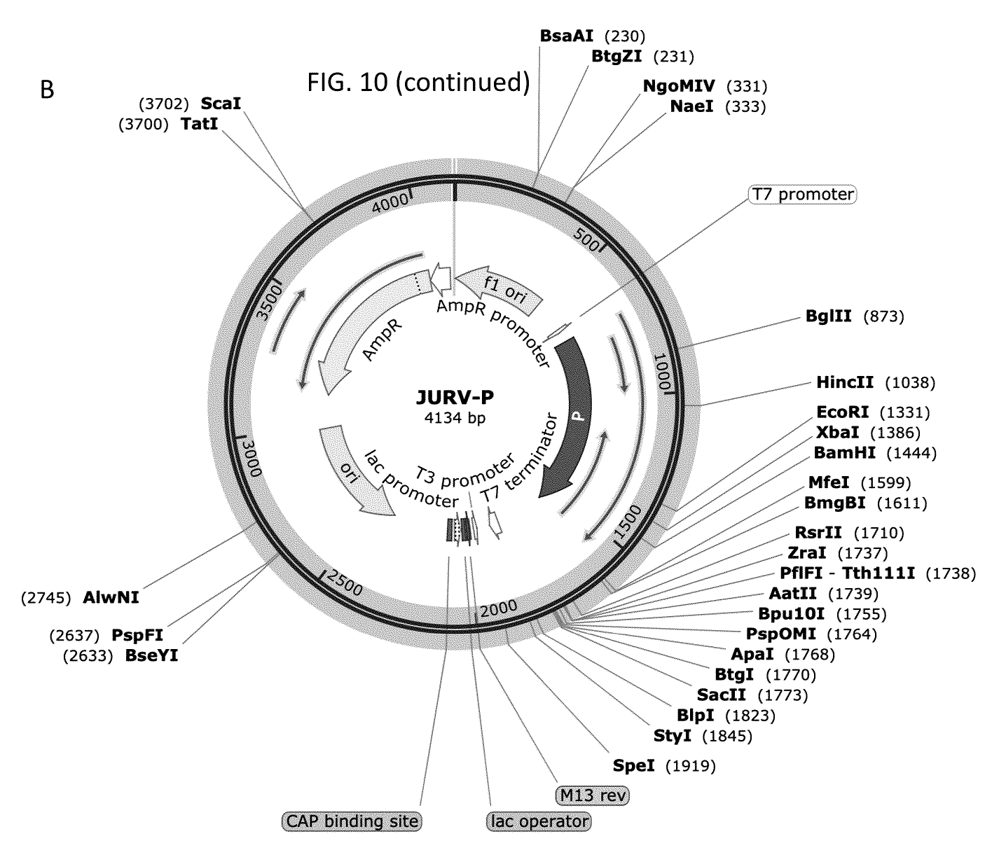

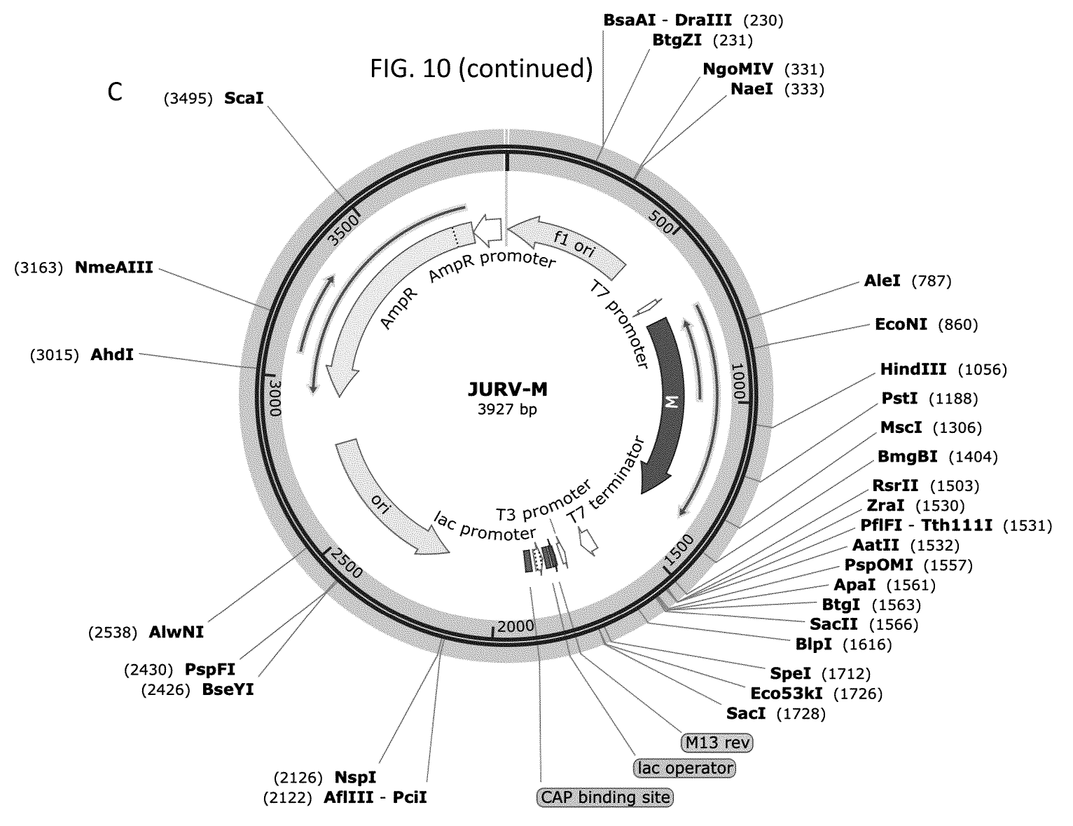

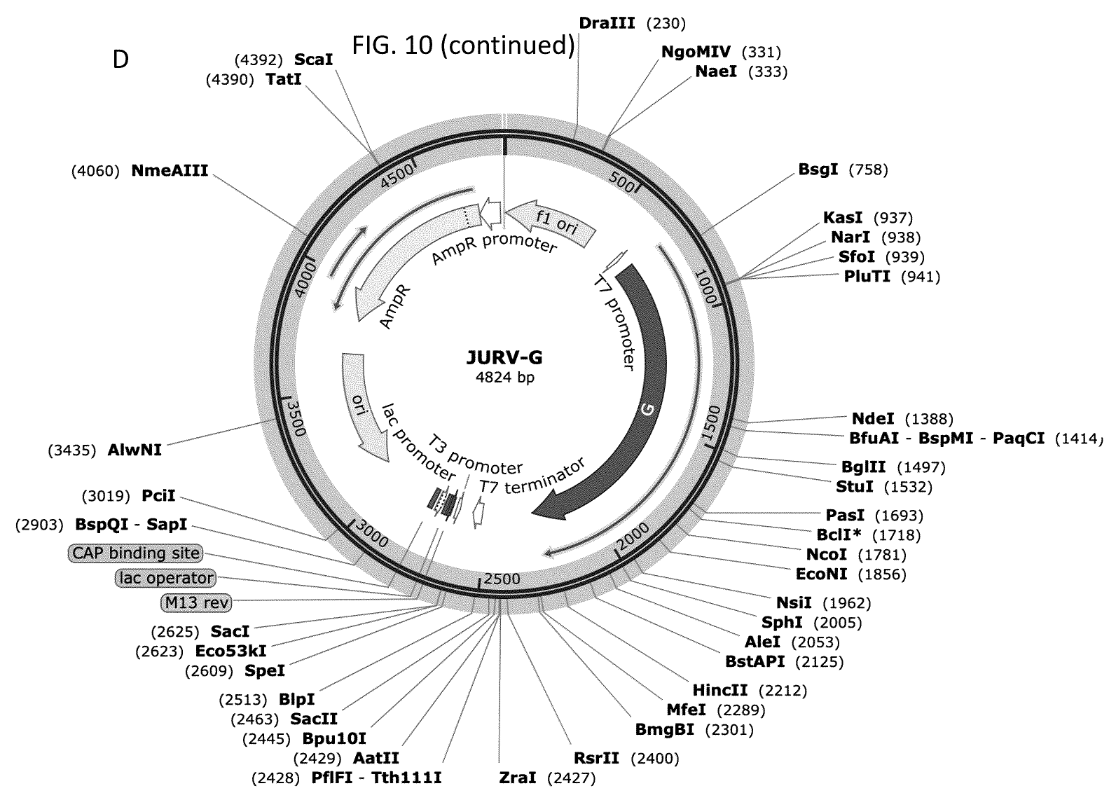

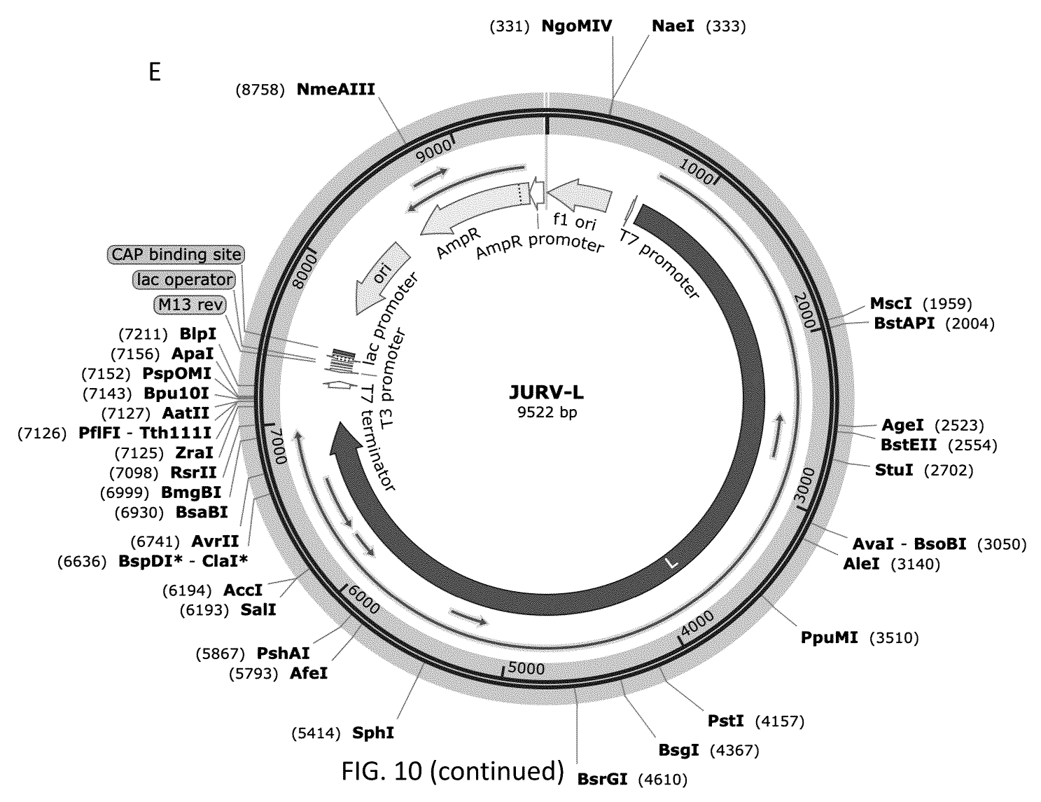

A representative JURV sequence comprises SEQ ID NO: 12 of the present disclosure, and a plasmid map JURV-XN-2 comprising said sequence (SEQ ID NO: 14) can be found in FIG. 8. A representative JURV sequence with green fluorescent protein fusion comprises SEQ ID NO: 13 of the present disclosure, and a plasmid map JURV-GFP comprising said sequence (SEQ ID NO: 15) can be found in FIG. 9. Plasmid maps for JURV-N(SEQ ID NO: 16), JURV-P (SEQ ID NO: 17), JURV-M (SEQ ID NO: 18), JURV-G (SEQ ID NO: 19), and JURV-L (SEQ ID NO: 20) can be found in FIG. 10A-E, respectively.

### Cell Viability Assays

For all cytotoxicity assays (96-well format), cells (1.5×10⁴) were infected with laboratory-based strain of JURV or MORV or VSV at MOI of 10, 1, and 0.1 in serum-free Gibco Minimum Essential Media (Opti-MEM). Cell viability was determined with Cell Titer 96 AQueous One Solution Cell Proliferation Assay. Data were generated as means of 6 replicates from 6 independent experiments, ±SEM.

### Visualization of Virus-Induced Cytopathic Effects in Cholangiocarcinoma Cells

JURV was used to infect adherent cells (5×10⁵ cells per well) in 6-well plates at MOI of 0.1. Cells were incubated at 37° C. until analysis. At 72 hours after infection, cells were fixed with 5% glutaraldehyde and stained with 0.1% crystal violet to visualize cellular morphology and remaining adherence, which indicates cell viability. Pictures of representative areas were taken.

### Animal Studies

Under a protocol approved by the Mayo Clinic Institutional Animal Care and Use Committees, we conducted the in vivo evaluations described below.

### Toxicology and Biodistribution of Virus Administrated Via the Nasal Route

To determine whether treatment with JURV could be associated with neurotoxicity or liver toxicity, female C57BL/6J mice (N=36; n=6 mice per group), including control, were intranasally (IN) or intravenously (IV) administered (25 μl per nostril) PBS or a dose (1×10⁷, 1×10⁸ TCID₅₀) of JURV. Body weight, temperature, behavior, and clinical signs were monitored by a board-certified veterinarian at least 3 times per week to detect signs of toxicity. Three days post-infection, 3 mice per group were sacrificed and tissues were collected (brain and liver) for evaluation of short-term toxicity and viral biodistribution. The remaining mice were monitored for 45 days, and body weights and clinical observations were recorded at least 3 times per week for the study duration.

### Blood Tests

Blood was collected from the submandibular vein (cheek bleed) on day 3 and from cardiac puncture on day 45. Blood was collected for complete blood counts in BD Microtainer tubes with ethylenediaminetetraacetic acid or lithium heparin (Becton, Dickinson and Company) and for serum analysis in BD Microtainer SST tubes (Becton, Dickinson, and Company). Analysis of complete blood counts was performed in an Piccolo Xpress chemistry analyzer (Abaxis), and blood chemistry analysis was done in a VetScan HM5 Hematology Analyzer (Abaxis).

### In Vivo Efficacy Study on Human CCA and HCC Xenograft Models

To evaluate the in vivo therapeutic efficacy of oncolytic JURV in subcutaneous xenograft models of murine tumors and human hepatocellular carcinoma, tumor cells (2×10⁶) were subcutaneously inoculated into the right flanks of female athymic nude (NU/J) mice (n=7 mice per group) (Jackson Laboratories). When tumors reached an average size of 80-120 mm³, mice were randomized into treatment groups and were dosed within 24 hours of randomization. Each mouse received 3 intratumoral injections (50 μl containing PBS or 1×10⁷ TCID₅₀ units of JURV or PBS), each 1 week apart. Tumor volume and body weight were monitored. Mice were euthanized when adverse effects were observed or when tumor size was larger than 2,000 mm³. Tumor volume was calculated with the following equation: (longest diameter*shortest diameter²)/2. Tumor images were taken before resection and tumor weights recorded after resection.

### Analysis of Tumor-Infiltrating Immune Cells

Upon excision, tumors from 5 mice per group were dissociated with gentleMACS Octo Dissociator (Miltenyi), according to the manufacturer's protocol. CD45⁺ cells were isolated with CD45 (TIL) mouse microbeads (Miltenyi). Cells were incubated with Fixable Viability Stain 510 (BD Horizon) for 15 minutes, followed by anti-Fc blocking reagent (Miltenyi) for 10 minutes prior to surface staining. Cells were stained, followed by data acquisition on a MACSQuant Analyzer 10 optical bench flow cytometer (Miltenyi). All antibodies were used according to the manufacturer's recommendation. Fluorescence Minus One control was used for each independent experiment to establish gating. For intracellular staining of granzyme B, cells were stained with the intracellular staining kit (Miltenyi). Analysis was performed with FlowJo (TreeStar). Forward scatter and side scatter were used to exclude cell debris and doublets.

### Flow Cytometry Analysis Antibodies

The following antibodies were used for flow cytometry analysis: CD45-FITC (Cat. #553079, BD Biosciences), CD3-BUV395 (Cat. #563565, BD Biosciences), CD4-BUV737 (Cat. #612761, BD Biosciences), CD8-Percp-Cy5.5 (Cat. #45-0081-82, eBioscience), CD44-BV711 (Cat. #103057, Biolegend), CD335-PE/Dazzle594 (Cat. #137630, Biolegend), PD-1-PE (Cat. #551892, BD Biosciences), Ki67*-BV605 (Cat. #652413, Biolegend), Granzyme B*-APC (Cat. #366408, Biolegend), IFN-γ*-BV421 (Cat. #563376, BD Biosciences), CD11b-PE-Cy7 (Cat. #101216, Biolegend), F4/80-BV510 (Cat. #123135, Biolegend), CD206-AF700 (Cat. #141734, Biolegend), I-A/I-E-BV786 (Cat. #743875, BD Biosciences), and L/D-efluor780 (Cat. #65-0865-18, eBioscience).

### Histopathological Analysis

Assessment of any abnormal changes in brain and liver was determined by histopathological evaluation of H&E-stained images, reviewed by a board-certified pathologist. HALO v3.1.1076.379 was used to measure the percentage tumor necrotic area.

### Statistical Analysis

All values were expressed as mean±standard deviation, and the results were analyzed by one-way analysis of variance, followed by the Tukey test for multiple comparisons and the Kaplan-Meier method for survival, using statistical software in GraphPad Prism, version 8 (GraphPad Software). A p value less than 0.05 was considered significant.

### REFERENCES

1. Walker, P. J., et al. Evolution of genome size and complexity in the rhabdoviridae. PLoS Pathog 11, e1004664 (2015).
2. Amarasinghe, G. K., et al. Taxonomy of the order Mononegavirales: update 2017. Arch Virol 162, 2493-2504 (2017).
3. Lawson, N. D., Stillman, E. A., Whitt, M. A. & Rose, J. K. Recombinant vesicular stomatitis viruses from DNA. Proc Natl Acad Sci USA 92, 4477-4481 (1995).
4. Whelan, S. P., Ball, L. A., Barr, J. N. & Wertz, G. T. Efficient recovery of infectious vesicular stomatitis virus entirely from cDNA clones. Proc Natl Acad Sci USA 92, 8388-8392 (1995).

### Example 2: Comprehensive Proteogenomic Analysis of the Anti-Tumor Immunoactivity of a Novel Oncolytic Vesiculovirus in Hepatocellular Carcinoma

In this example, we show that intratumorally (IT) administration of Jurona virus (JURV) induces dynamic tumor regression in human HCC xenograft and syngeneic models. Furthermore, IT injections of JURV elicited the recruitment and activation of cytotoxic T lymphocytes (CTLs) and decreased the tumor-associated macrophage (TAM) infiltration leading to tumor growth delay in both local and distant murine HCC tumors in a syngeneic model. Moreover, when administered concomitantly, JURV and anti-PD-1 antibodies synergized to modulate the tumor microenvironment (TME) via an increase in tumor-infiltrating CD4+ T cells and depletion of CD8+ PD-1+ and NK cells. Mechanistically, our analysis unveiled that JURV, and anti-PD-1 antibodies activate different effectors of the immune system but have complementary anti-tumor activities. Besides, our results indicate that the abscopal effect induced by JURV is likely mediated by the activation of several tumor suppressor genes and the mechanism regulating the T helper cell responses. Our work supports the further development of JURV as a novel immunovirotherapy platform for hepatocellular carcinoma.

### Results

### In Vitro Cytotoxicity Activity of JURV in HCC Cells

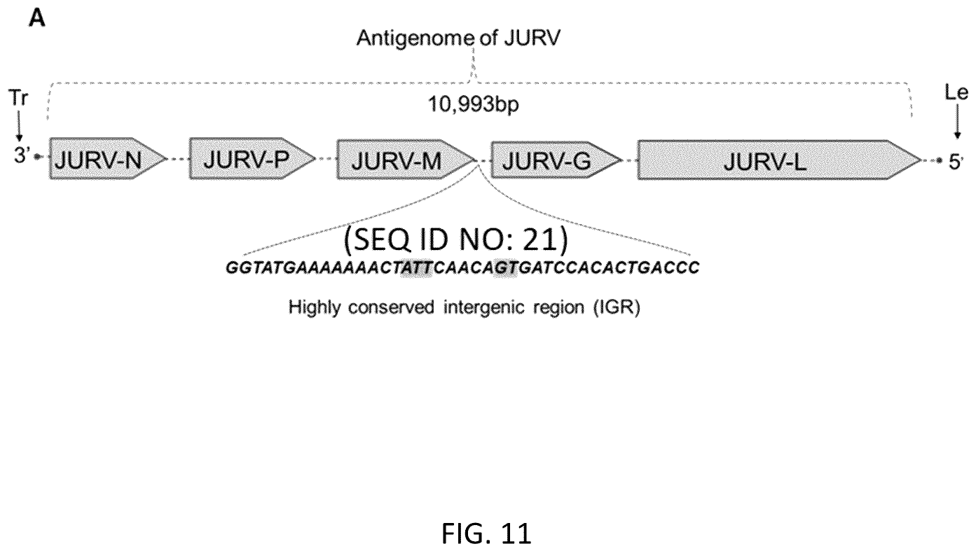

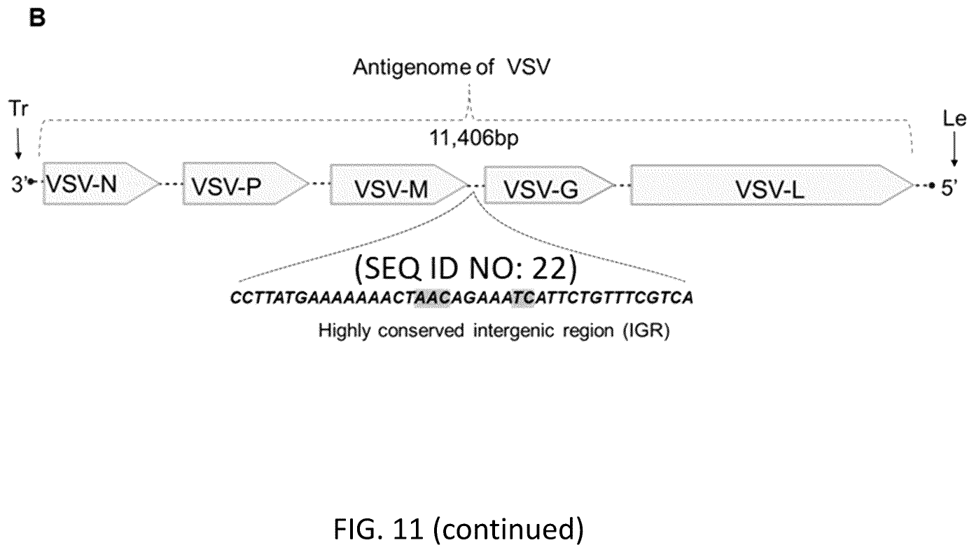

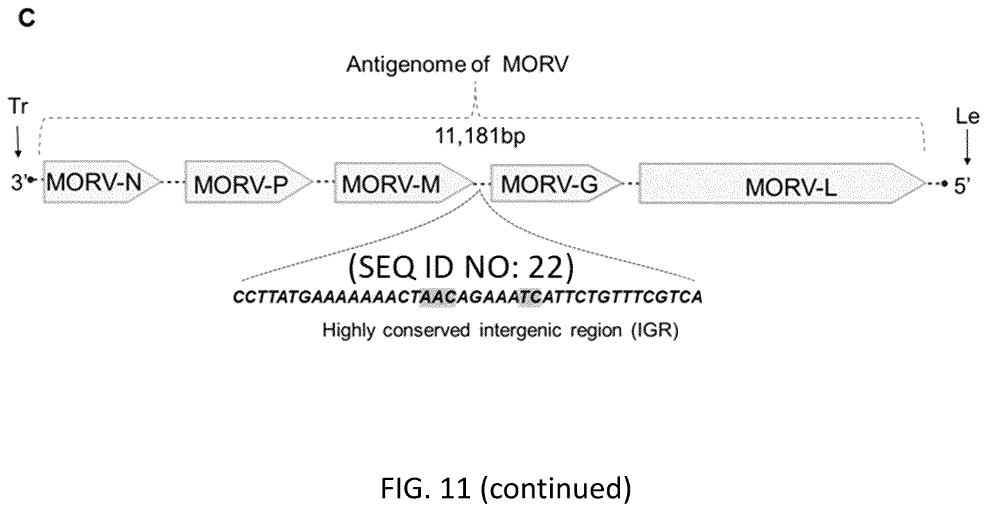

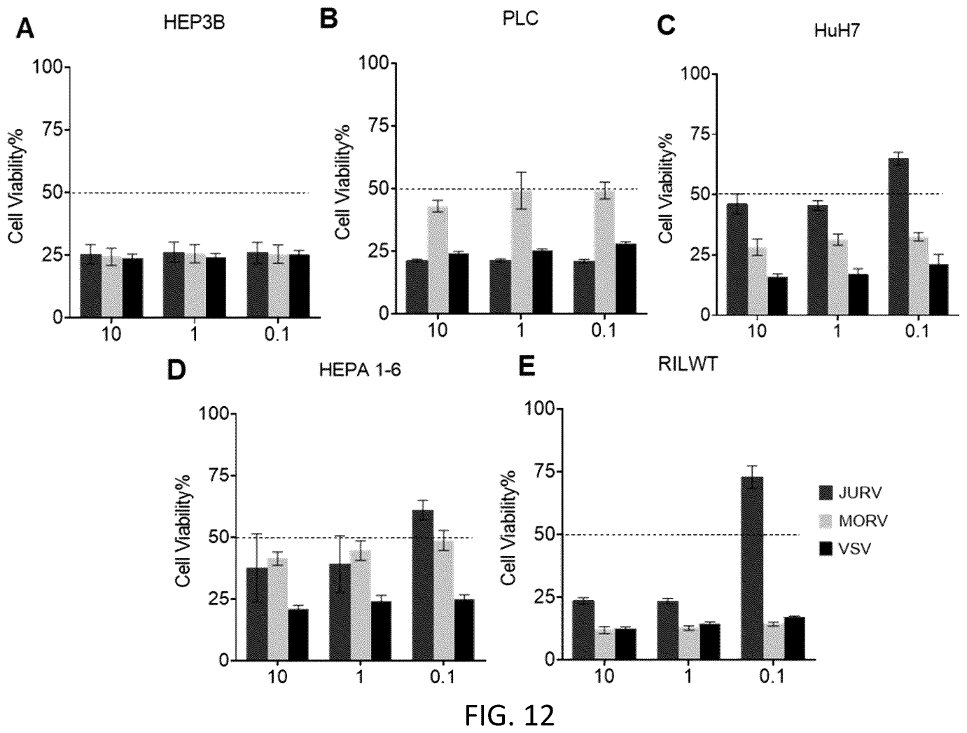

Analysis of the genome of Jurona virus (JURV) showed an identical genome organization with that of vesicular stomatitis virus (VSV) and morreton virus (MORV) (FIG. 11), two other members of the Rhabdoviridae⁶ family previously investigated for their oncolytic potential in human cancers.⁷ To investigate whether JURV possesses similar cancer-cell killing ability in vitro, we infected monolayers of human and murine HCC cells, including HEP3B, PLC, HuH7, HEPA 1-6, and RILWT with JURV, VSV, or MORV, respectively at a multiplicity of infection (MOI) of 0.1, 1 or 10 (FIG. 12A-E). We performed an MTS cell viability assay assessment of cytotoxic at 72 hours post-infection to determine susceptibility to virus infection and the extent of virus-induced cancer cell death. Cell viability assay results are summarized in Table 1. Irrespective of the MOIs, a substantial decrease in cell viability (˜30%) was observed for HEP3B cells in response to infection with JURV, VSV, and MORV (FIG. 12A). A difference in virus-induced cell death was also noted in PLC, HuH7, HEPA 1-6, and RILWT cells (FIG. 12B-E). In PLC cells, while JURV and VSV induced similar cytotoxicity rates (˜25%) at MOIs of 10, 1, and 0.1, cell viability in MORV-infected cells was twice higher (˜50%). HuH7, HEPA 1-6, and RILWT cells displayed lower sensitivity to the JURV infection than MORV and VSV, except for RILWT cells treated with JURV at MOI of 1 and 10 (˜24%). We subsequently infected HCC cells with JURV at an MOI of 0.1. Three days after incubation, cells were stained with crystal violet to assess the comparative cytotoxic effect of JURV in infected cancer cells. We observed that while HuH7 had residual (<50%) live adherent cells remaining after infection, HEP3B, PLC, HEPA 1-6, and RILWT (>90%) had a complete loss of adherent cells, suggesting that MTS may have slightly underestimated the oncolysis effect of JURV (See FIG. 3E). Furthermore, analysis of JURV kinetics showed a 1000-fold increase in viral titers around 10 hours post-infection (See FIG. 3E), indicating a high permissivity and robust replication capability of JURV in HCC cells. Our results indicate that JURV, VSV, and MORV can efficiently infect and lyse murine and human HCC cells. However, the basis for the differences observed in HCC cell-killing mediated by these viruses is worth investigating in future studies.

TABLE 1
In vitro cell viability assay results.

| Cells | MOI | Viruses | Viability (%) |
|---|---|---|---|
| HEP3B | 0.1 | JURV | 31.59 |
| | | MORV | 29.80 |
| | | VSV | 26.68 |
| | 1 | JURV | 29.01 |
| | | MORV | 28.75 |
| | | VSV | 25.84 |
| | 10 | JURV | 28.78 |
| | | MORV | 27.43 |
| | | VSV | 25.26 |
| PLC | 0.1 | JURV | 23.41 |
| | | MORV | 46.20 |
| | | VSV | 28.67 |
| | 1 | JURV | 21.34 |
| | | MORV | 53.67 |
| | | VSV | 25.86 |
| | 10 | JURV | 20.43 |
| | | MORV | 43.42 |
| | | VSV | 24.47 |
| HuH7 | 0.1 | JURV | 54.45 |
| | | MORV | 33.34 |
| | | VSV | 26.91 |
| | 1 | JURV | 45.14 |
| | | MORV | 32.90 |
| | | VSV | 18.30 |
| | 10 | JURV | 44.62 |
| | | MORV | 28.35 |
| | | VSV | 16.78 |
| HEPA1-6 | 0.1 | JURV | 48.97 |
| | | MORV | 48.94 |
| | | VSV | 26.34 |
| | 1 | JURV | 44.03 |
| | | MORV | 44.99 |
| | | VSV | 25.67 |
| | 10 | JURV | 26.64 |
| | | MORV | 43.51 |
| | | VSV | 22.06 |
| RILWT | 0.1 | JURV | 49.86 |
| | | MORV | 14.98 |
| | | VSV | 17.58 |
| | 1 | JURV | 24.20 |
| | | MORV | 11.86 |
| | | VSV | 14.34 |
| | 10 | JURV | 20.94 |
| | | MORV | 11.20 |
| | | VSV | 12.54 |

In Vitro Cell Viability of JURV-Infected HCC Cells Pretreated with Type IIFN-α

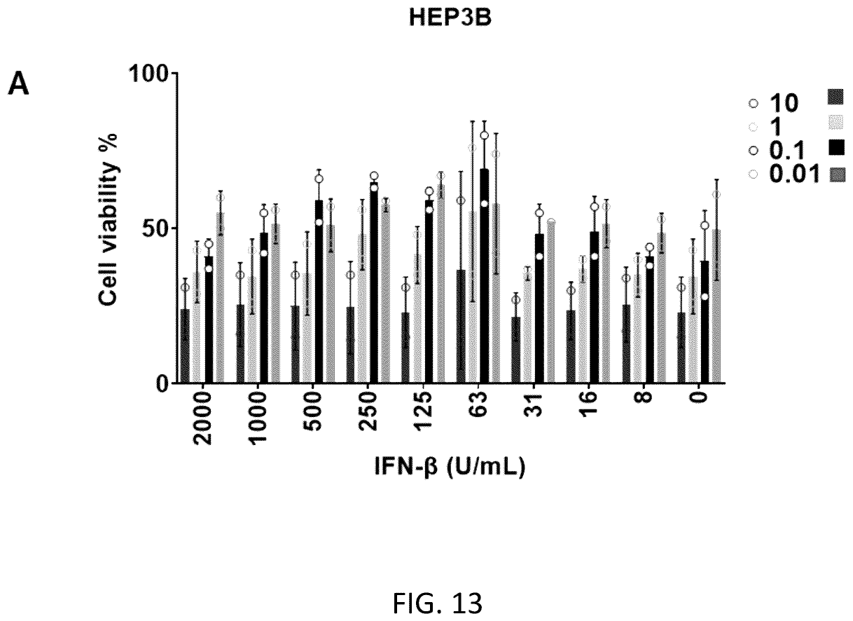

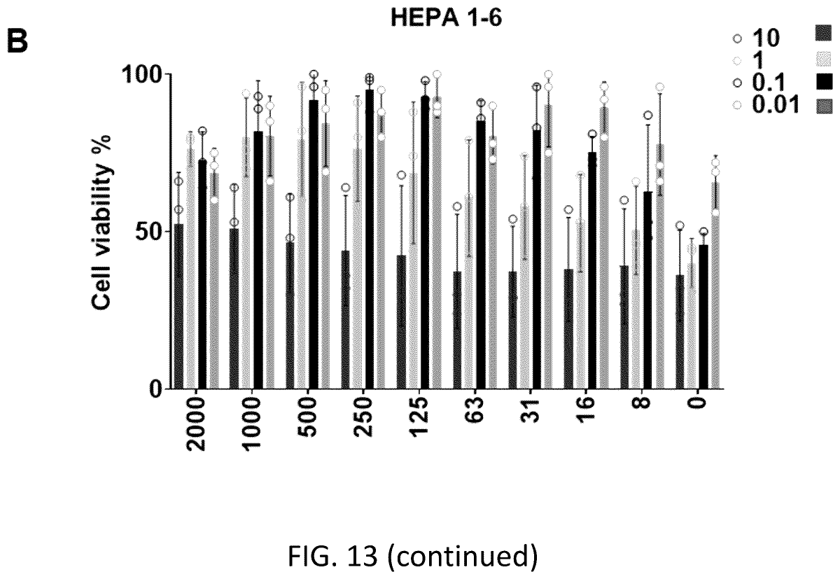

The tumor microenvironment (TME) is home to complex interactions between cancer cells, healthy tissues, and diverse components of the immune system.⁸ Detection of viral pathogen associated molecular patterns (PAMPs) by pattern recognition receptors (PRRs) present in most immune cells induce the production of numerous cytokines including IFNα/β that activate hundreds of interferon-stimulated genes (ISGs).^{9,10} The activation of ISGs triggers innate antiviral mechanisms and generates an adaptive cellular response to virus infection. Interestingly, studies have shown that the type I IFN signaling pathway defects often coincide with carcinogenesis and create needful conditions for tumor-selective viral replication and oncolysis of many oncolytic viruses (OVs).^{11,12} However, upon sensing the presence of viruses, immune cells and non-cancer cells in the TME can produce type I IFN-α/β. This could prematurely impair oncolytic activity of OVs if the tumor cells are responsive to the effect of exogenous IFNs.^{11,13} Therefore to determine the impact of type I IFN on the outcome of JURV infection, we compared the susceptibility to JURV infection of monolayers of human (HEP3B) and murine HCC (HEPA 1-6) cells pretreated with species-specific IFN-α. Specifically, we treated HEP3B and HEPA 1-6 cells with serial concentrations of IFN-α followed with infection with JURV at MOI of 10, 0.1, or 0.01, as indicated in Methods and FIG. 13A. Cell viability (MTS) was assessed seventy-two hours post-infection. Our data indicate that treatment with IFN-α did not shield (30-75% of cell death) human HCC (HEP3B) and mouse HCC (HEPA 1-6) cells from JURV-induced cytopathic effect. However, we noted a difference in response to IFN-α between the treated HCC cells. At an MOI of 1, a concentration of 500 U/mL IFN-α killed approximately 20% of HEPA 1-6 (FIG. 13B). In contrast, the same treatment yielded ˜75% of cell killing in HEP3B cells (FIG. 13A). Overall, there was no statistical difference in cell viability among different treatment groups, suggesting that treatments with IFN-α does not significantly alter JURV oncolytic activity and that the IFN pathway might be defective in these HCC cells as indicated in our previous works.^{7,14}

### In Vivo Safety Assessment of JURV

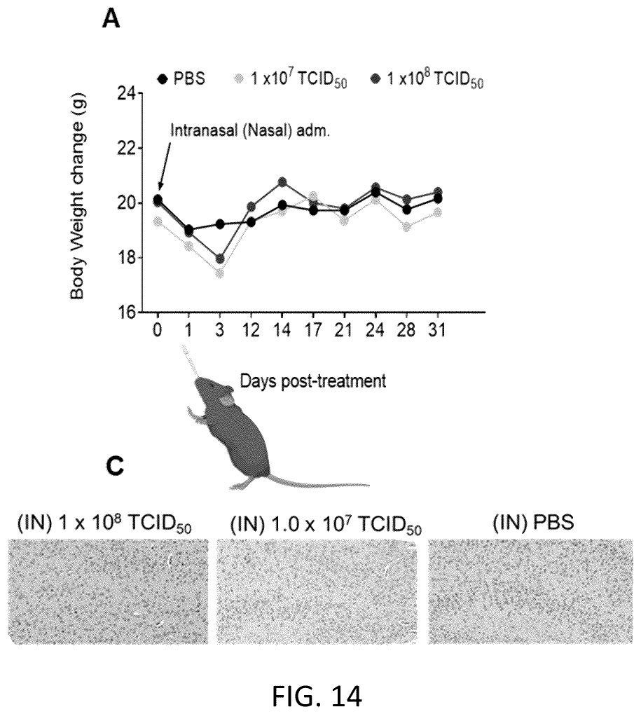

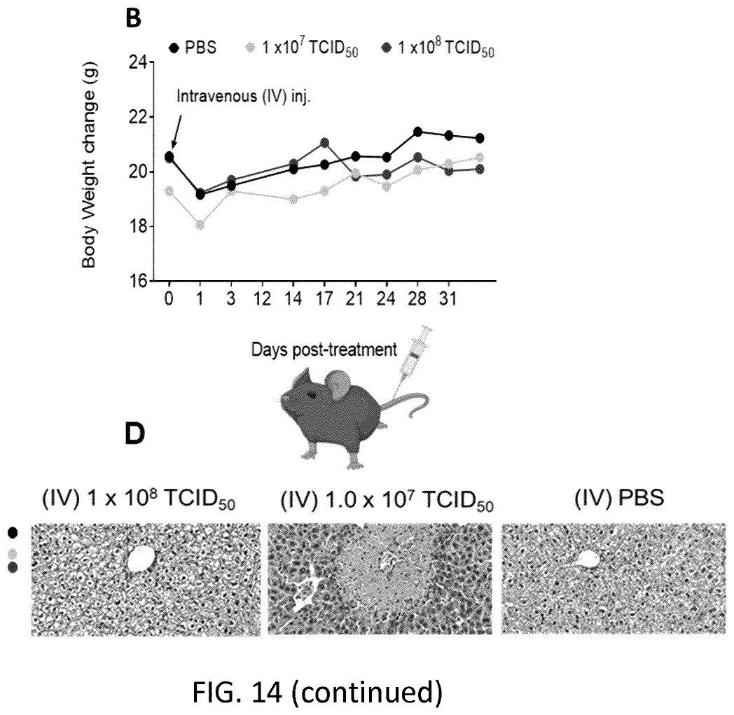

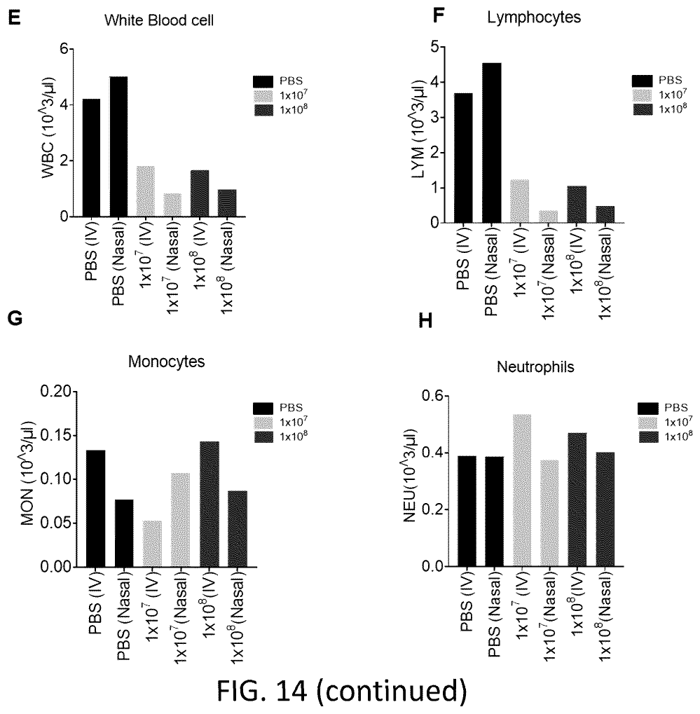

Reports have indicated that intranasal (IN) administration of wild-type VSV in mice results in significant weight loss and lethality at around three days post-infection.¹⁵⁻¹⁷ Because JURV shares the same genomic structure as VSV, we sought to determine whether acute adverse events may result from infection with JURV in mice, as described for VSV and VSV-derived vectors.¹⁸⁻²⁰ To increase the likelihood of observing adverse events, we selected two doses of wild type JURV that are 10 to 100-fold higher than the dose of 1×10⁶ TCID₅₀ associated with toxicity in wild-type VSV-infected mice¹⁵⁻¹⁷ Non-tumor bearing healthy laboratory mice were administered with JURV at a dose of 1×10⁷ or 1×10⁸ TCID₅₀ intranasally or intravenously (IV) to mimic the natural route of systemic VSV infection. As control groups, we administered PBS to the animals parallelly. At three days post-infection, halves of the mice were sacrificed, and blood and animal tissues (brain, liver, and spleen) were harvested and subjected to H&E staining to assess short-term toxicity. In the virus-treated groups, mice weights dropped by 10-15% on day 3 compared to the control groups (FIGS. 14A and B). However, an expert review of histology slides did not find an obvious abnormality in the brain, liver, or spleen sections of animals treated with either low- or high-dose of JURV cohorts (FIGS. 14C and D). There was transient leukopenia in mice treated with JURV, including a decrease in white blood cells and lymphocytes (FIG. 14E-H). Virus infection is characterized by leukopenia in humans and animals and has been well-documented for systemic VSV infection.²¹ Our data did not indicate a significant difference in body weights or clinical signs (i.e., paralysis, death, matted fur) in mice infected with JURV as compared to PBS treated.

### Toxicoproteomics Analysis of JURV

As discussed above, no change in brain and liver tissues harvested at three days post-infection with JURV indicated severe toxicity, including neurotoxicity or hepatotoxicity at any dose of the virus tested. Next, we performed quantitative proteomics analysis of these tissues to identify potential biological changes associated with neuroprotection and antihepatotoxicity in mice infected with high doses of JURV. We used Ingenuity Pathway Analysis (IPA) to identify enriched biological processes and signaling pathways of differentially expressed proteins (DEPs) with significantly altered expression in the brains and livers of mice infected with JURV.

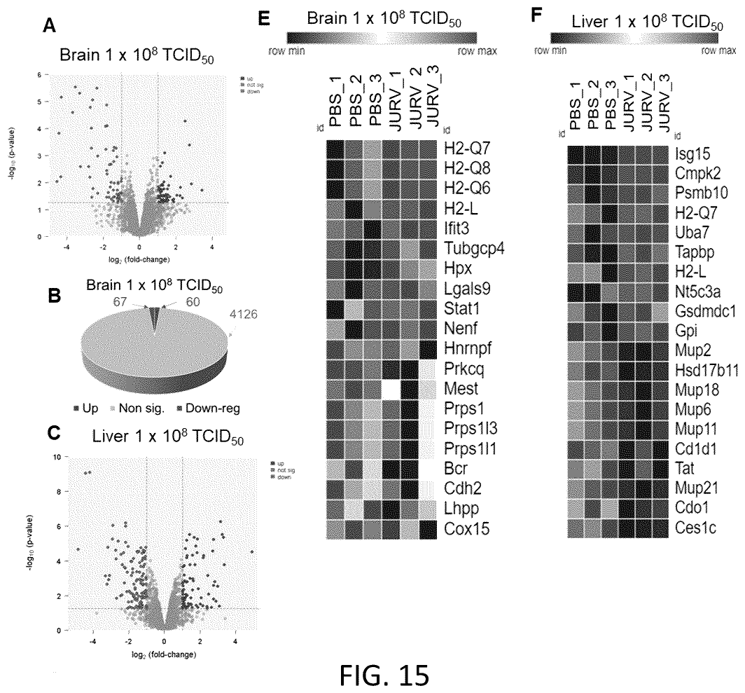

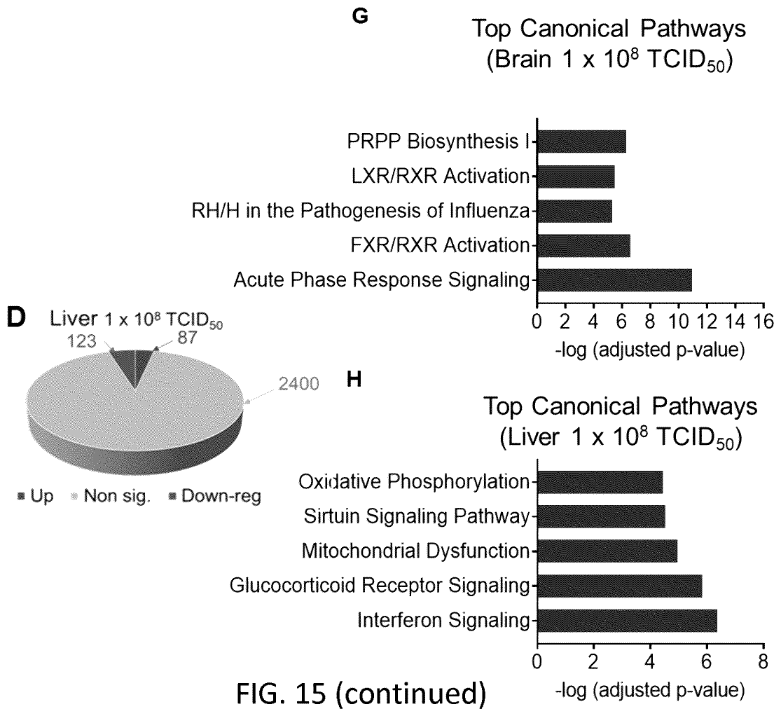

Among 4,253 analyzed DEPs in the brain of mice treated with 1.0×10⁸ TCID₅₀ of JURV compared to control, 127 DEPs had significantly altered expressions (2-fold change >2, p-value <0.055). These DEPs comprise 60 upregulated, and 67 downregulated DEPs (FIGS. 15A and B). Among 2,400 analyzed DEPs in the liver from the same mice, 87 DEPs were upregulated, while 123 DEPs were downregulated (FIGS. 15 C and D). To understand the dynamics of biological changes in these tissues, we further scrutinized the top ten upregulated and downregulated DEPs (FIG. 15E) and the five most significant signaling pathways in the brain (FIG. 15G). Using the National Library of Medicine (NLM) database, we matched these proteins with their human orthologs, except Prps1l3 (Table 2), which allowed us to gain insights into the changes in protein expression in response to the JURV infection.

TABLE 2
List of DEPs up and down-regulated in the brain.

| Official | Official Full | Common | Expression | | |
|---|---|---|---|---|---|
| Symbol | Name | name | Level | P-value | Human ortholog |
| H2-Q7 | Histocompatibility | Q9; Ped; Qa7; Qa- | −3.537326805 | 0.00346111 | HLA-E; HLA-F; |
| | 2, Q region locus 7 | 2; Qa-7; H-2Q7 | | | HLA-G |
| H2-Q8 | Histocompatibility | Qa8; Qa-2; Qa-8; H- | −3.537326805 | 0.00346111 | HLA-A; HLA-B; |
| | 2, Q region locus 8 | 2Q8; Ms10t; | | | HLA-C |
| | | MMS10-T | | | |
| H2-Q6 | histocompatibility | Qa6; Qa-6; H-2Q6; | −3.537326805 | 0.00346111 | HLA-E; HLA-F; |
| | 2, Q region locus 6 | H2-K1; | | | HLA-G |
| | | 0610037M15Rik | | | |
| H2-L | histocompatibility | H-2L | −2.356805655 | 0.00346111 | Part of MHC class |
| | 2, D region locus L | | | | I protein complex |
| IFIT3 | interferon induced | P60; IRG2; IFI60; | | | interferon-induced |
| | protein with | IFIT4; ISG60; RIG- | | | protein with |
| | tetratricopeptide | G; cig41; CIG-49; | | | tetratricopeptide |
| | repeats 3 | GARG-49 | | | repeats 3 isoform |
| Tubgcp4 | tubulin, gamma | 76p; GCP-4; | −4.321812728 | 0.004995018 | tubulin γ complex |
| | complex | D2Ertd435e; | | | associated protein |
| | associated protein 4 | 4932441P04Rik | | | 4 |
| Hpx | hemopexin | hx; Hpxn | −2.595448845 | 0.005385906 | hemopexin |
| Lgals9 | lectin, galactose | gal-9; Lgals5; | −1.811951482 | 0.007290906 | LGALS9 |
| | binding, soluble 9 | LGALS35; A407335; | | | |
| | | AI194909; | | | |
| | | AI265545; | | | |
| | | galectin-9 | | | |
| Stat1 | Signal transducer | AA408197; | −2.725157055 | 0.007934603 | STAT1 |
| | and activator of | 2010005J02Rik | | | |
| | transcription 1 | | | | |
| Nenf | Neuron derived | Spuf; SCIRP10; | −3.699902455 | 0.010900871 | NENF |
| | neurotrophic factor | 1110060M21Rik | | | |
| Hnrnpf | heterogeneous | Hnrpf; AA407306; | 2.714873369 | 0.110123442 | HNRNPF |
| | nuclear | 4833420I20Rik | | | |
| | ribonucleoprotein | | | | |
| | F | | | | |
| Prkcq | protein kinase C, | Pkcq; PKC-0; | 1.230786834 | 0.248854564 | PRKCQ |
| | theta | AW494342; PKCtheta; | | | |
| | | A130035A12Rik | | | |
| Mest | Mesoderm specific | Peg1; AA408879; | 1.133666492 | 0.270149463 | MEST |
| | transcript | AI256745 | | | |
| Prps1 | Phosphoribosyl | PRS-I; C76571; | 1.692467998 | 0.44825422 | PRPS1 |
| | pyrophosphate | C76678; Prps-1; | | | |
| | synthetase 1 | 2310010D17Rik | | | |
| Prps1l3 | Phosphoribosyl | Gm5081; | 1.692467998 | 0.44825422 | No ortholog |
| | pyrophosphate | AU021838; | | | |
| | synthetase 1-like 3 | AW536152 | | | |
| Prps1l1 | Phosphoribosyl | 1700011K15Rik | 1.692467998 | 0.44825422 | PRPS1L1 |
| | pyrophosphate | | | | |
| | synthetase 1-like 1 | | | | |
| Bcr | BCR activator of | AI561783; | 1.243319713 | 0.597290707 | BCR activator of |
| | RhoGEF and | AI853148; | | | RhoGEF and |
| | GTPase | mKIAA3017; | | | GTPase |
| | | 5133400C09Rik | | | |
| Cdh2 | Cadherin 2 | CDHN; Ncad; N- | 1.256785536 | 0.622849687 | CDH2 |
| | | CAD | | | |
| Lhpp | Phospholysine | 2310007H09Rik | 1.578262377 | 0.622849687 | LHPP |
| | phosphohistidine | | | | |
| | inorganic | | | | |
| | pyrophosphate | | | | |
| Cox15 | Cytochrome c | 2900026G05Rik | 1.14754639 | 0.622849687 | cytochrome c |
| | oxidase assembly | | | | oxidase assembly |
| | protein 15 | | | | homolog COX15 |

The gamma complex associated protein 4 (Tubgecp4), functioning in microtubule (MT) nucleation at the centrosome, was the most significantly upregulated protein in the brains of JURV-treated mice (FIG. 15E). Interestingly, studies have shown that alterations of MTs are essential for forming viral intracellular replication compartments²², suggesting that JURV induced MTs change in brain cells to favor virus replication. However, other upregulated proteins also included H2-Q7, H2-Q8, H2-Q6, and H2-L, the orthologs of human leukocyte antigen (HLA) complex molecules, HLA-A, —B, -C, -E, -G, and -F (Table 2). Several studies have showed that IFN-7 produced during viral infection upregulates HLA-A, —B, -C expressions in mammalians.^{23,24} HLA-A, —B, and -C specializes in antigen presentation to cytotoxic T cells; thus, are vital components of the cell-mediated adaptive immune responses against pathogens, including viruses and bacteria. In contrast, HLA-E, -G, and -F was reported to downregulate inflammatory and immune responses.^{25,26}

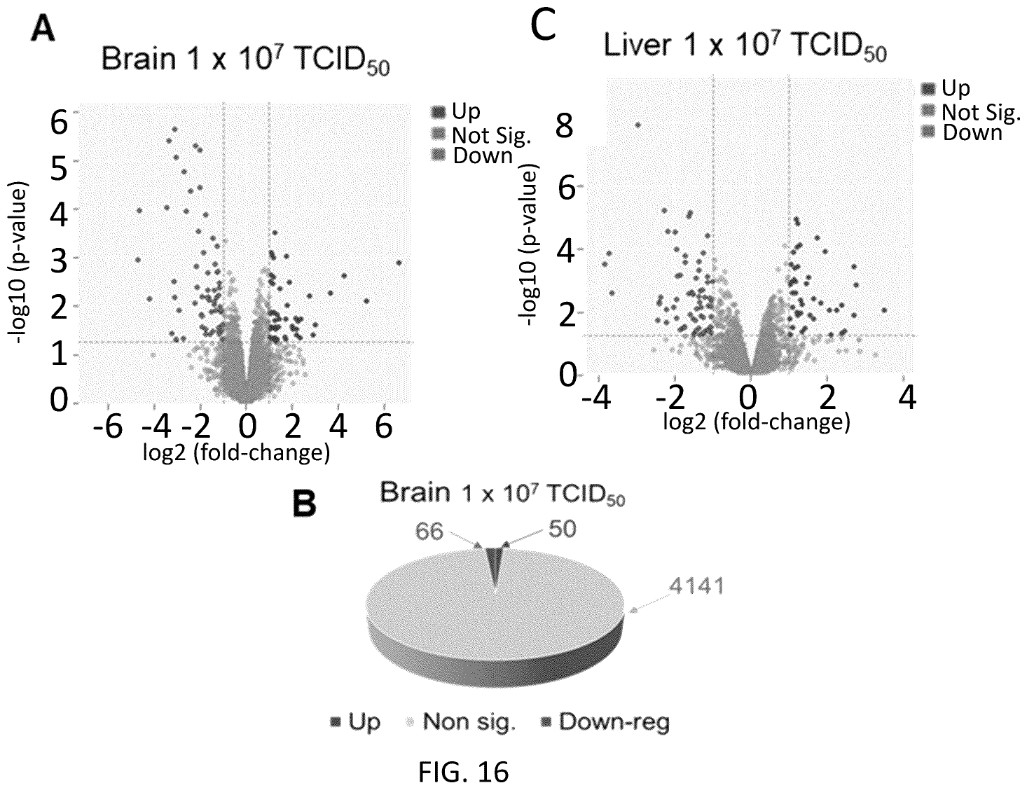

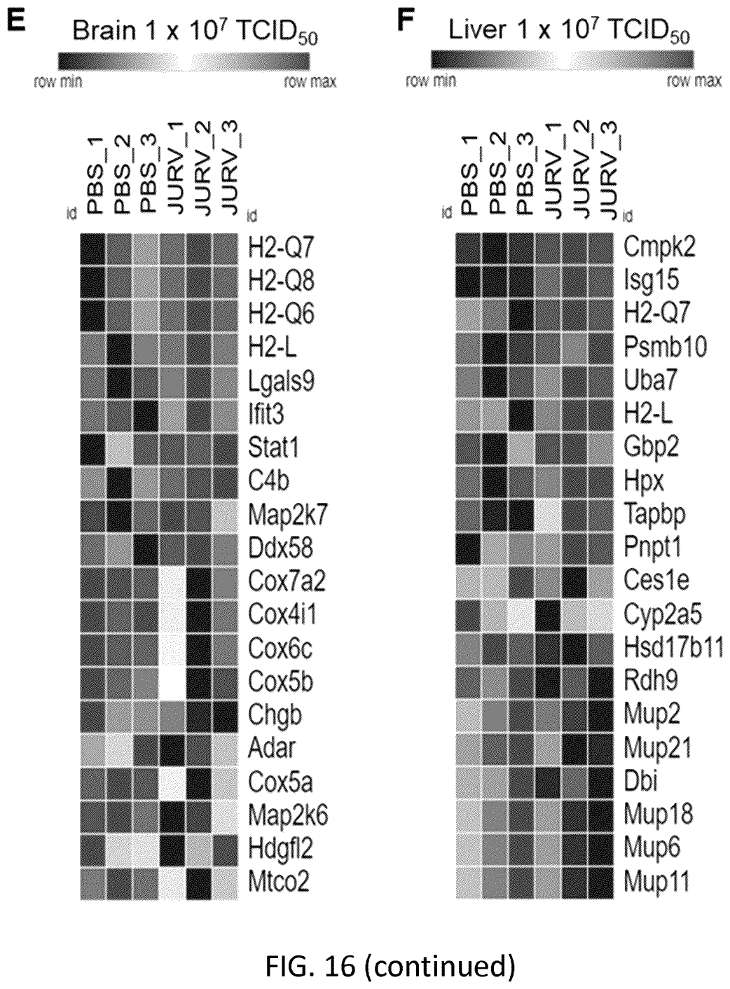

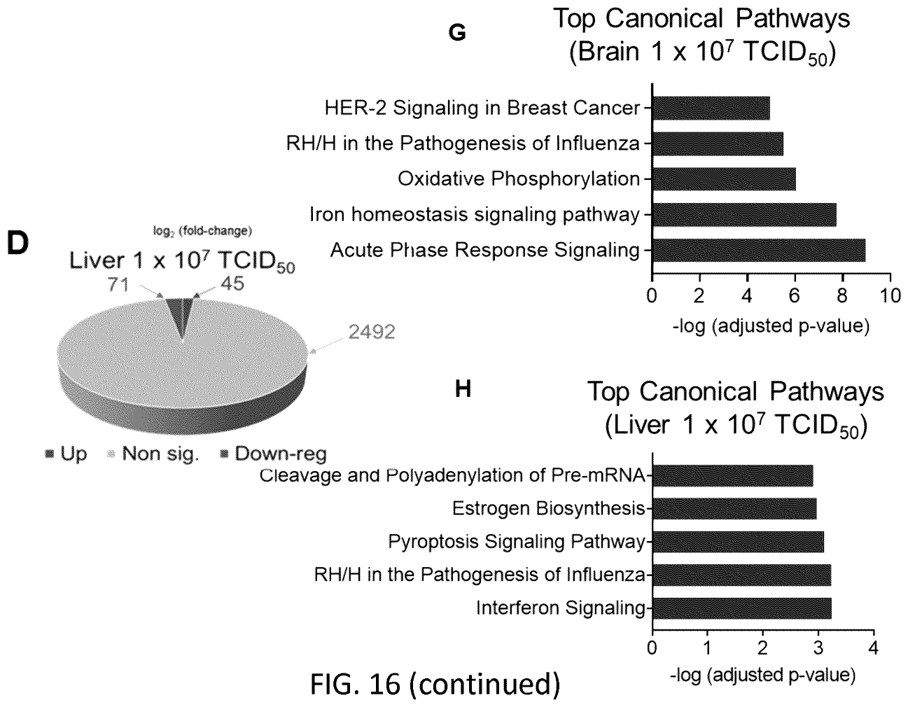

These findings imply that the host immune response in the brain after administration of a high dose of JURV was well toned, and viral infection was rapidly and effectively resolved. Surprisingly, we have found among the top-upregulated proteins, IFIT3 (interferon-induced protein with tetratricopeptide repeats 3)^{27,28} and Stat1 (Signal transducer and activator of transcription 1), both proteins have been shown to potentiate immune responses; however, Lgals9 (Lectin, galactose binding, soluble9) and HPx (Hemopexin) act as suppressors of excessive inflammatory and immune responses.^{29,30} In addition, Nenf (neurotrophic factor), which functions in neuron protections³¹⁻³³, was also upregulated in JURV-treated brain tissues. The predicted five most enriched signaling pathways were PRPP biosynthesis, LXR/RXR activation, RH/H in the pathogenesis of influenza, FXR/RXR activation, and acute phase response signaling (FIG. 15G). Based on the altered expression of DEPs used in the IPA analysis, we propose that activation of RH/H in the pathogenesis of influenza and acute phase response signaling occurred initially, leading to active PRPP biosynthesis essential to establish immunity against viruses. In contrast, activation of LXR/RXR and FXR/RXR seems to indicate an initial response of the infected mice to return to homeostasis. Similarly, the top 10 DEPs upregulated in the liver, such as Isg15³⁴, Cmpk2³⁵, Uba7³⁶, exhibit key cellular antiviral functions (FIG. 15F). IPA analysis of the DEPs in the liver (FIG. 15H) infected with JURV also predicted activation of the interferon signaling, mitochondrial dysfunction, acute phase response signaling pathways which are all related to sensing and induction of innate and adaptive immune response to viral infection. We found that changes in the DEPs following administration of 1×10⁷ TCID₅₀ of JURV were similar to that of a 10-fold higher dose (1×10⁸ TCID₅₀) of the virus (FIG. 16A-D). Moreover, analysis of the top 10 DEPs upregulated in the brain (FIG. 16E) and liver (FIG. 16G) and predicted activated pathways (IPA) are all immune responses to viral infection-related proteins and pathways (FIGS. 16G and H). Taken together these data strongly indicate that multiple mechanisms co-existed to control and clear JURV infection and simultaneously prevent neuron damage. These results indicate that the host cellular apparatus sensed JURV and that multiple mechanisms co-existed to control viral infection and clear the virus from the brain while simultaneously preventing neuron damage. In addition, it demonstrates that intranasal administrations of high doses of JURV did not elicit severe neurotoxicity or result in physical impairment in these mice.

### In Vivo Antitumor Efficacy of JURV in Syngeneic HCC Model

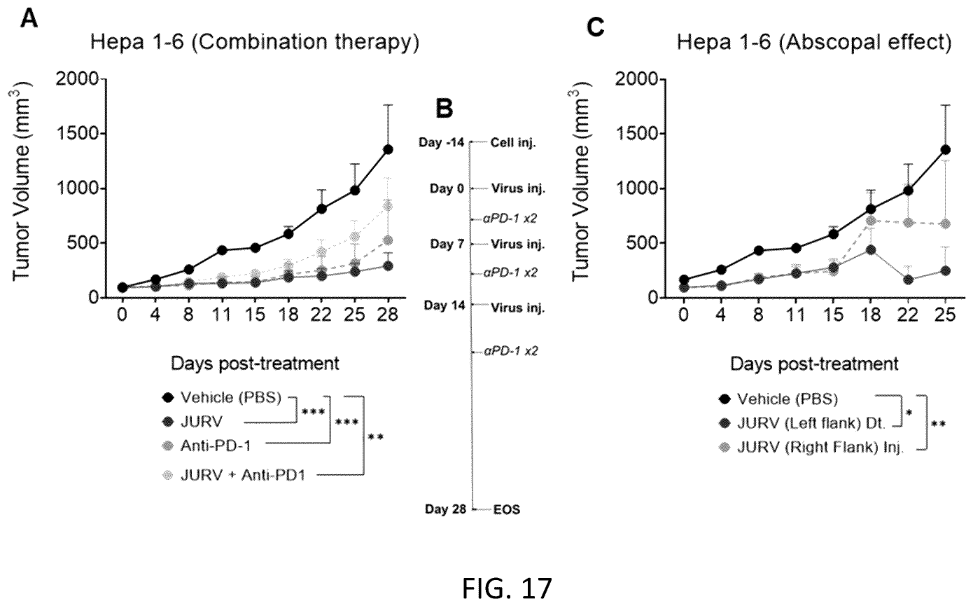

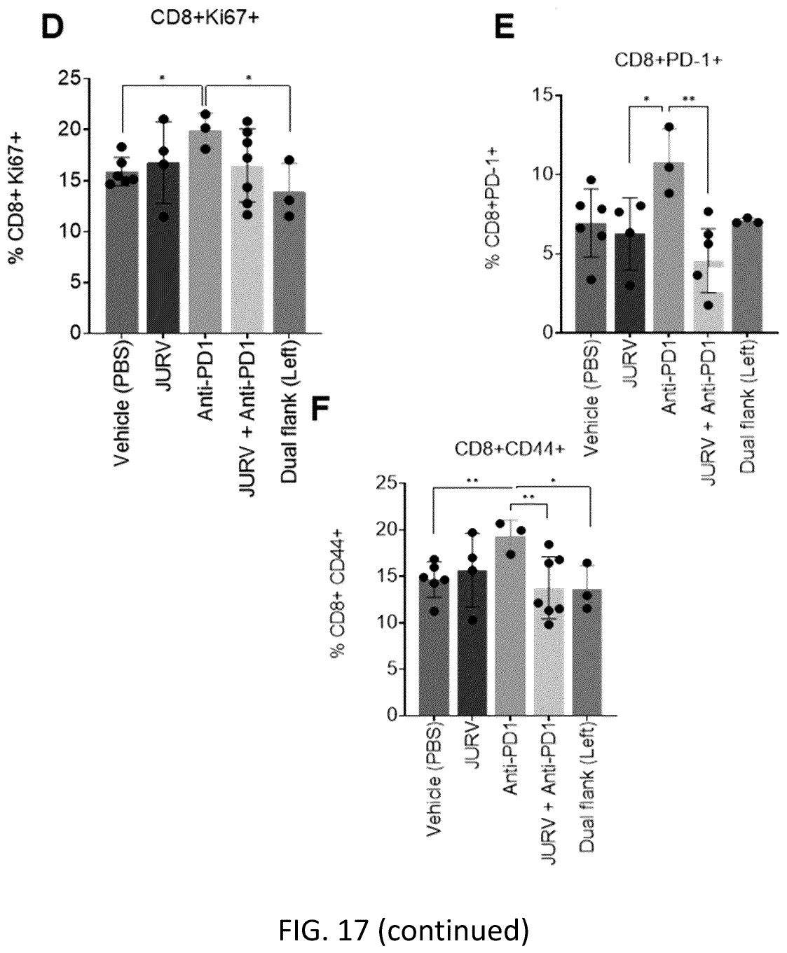

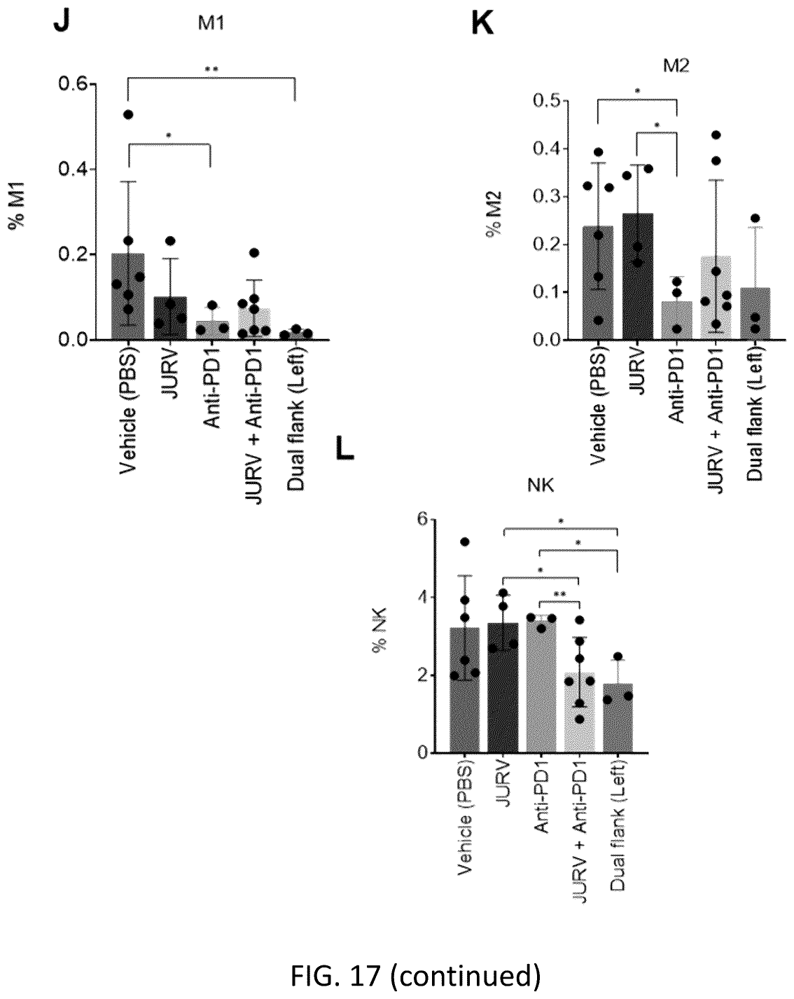

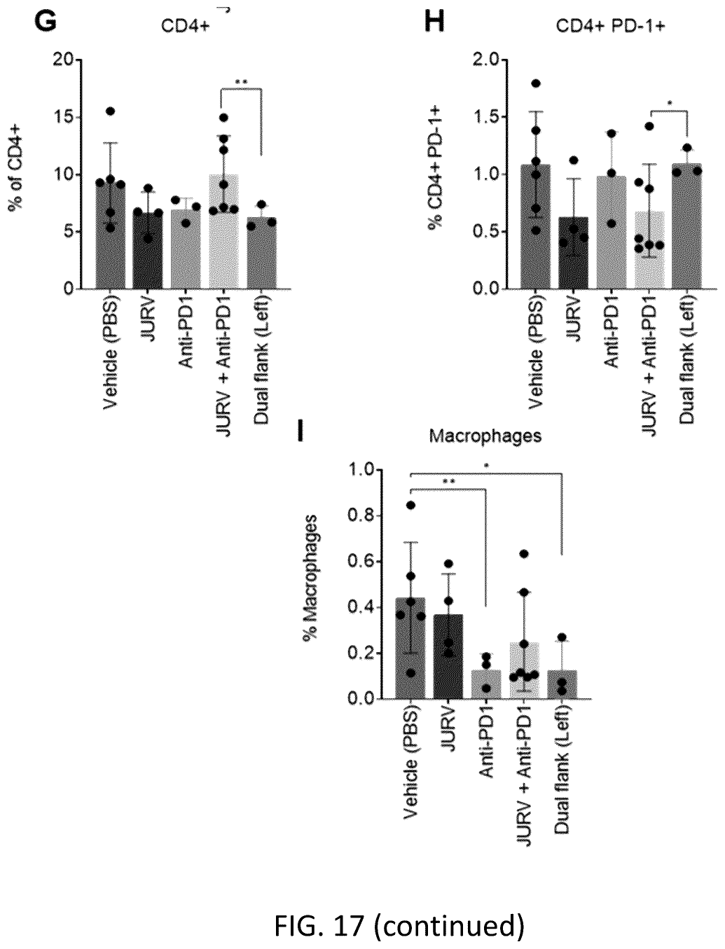

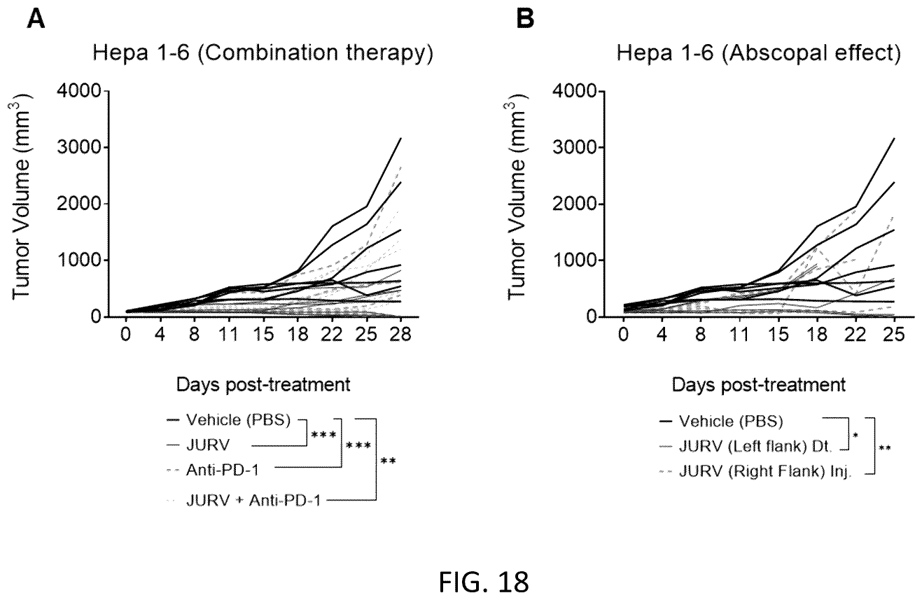

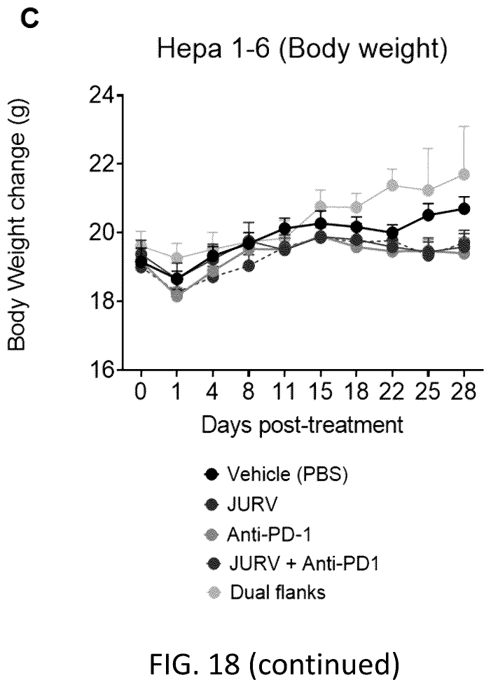

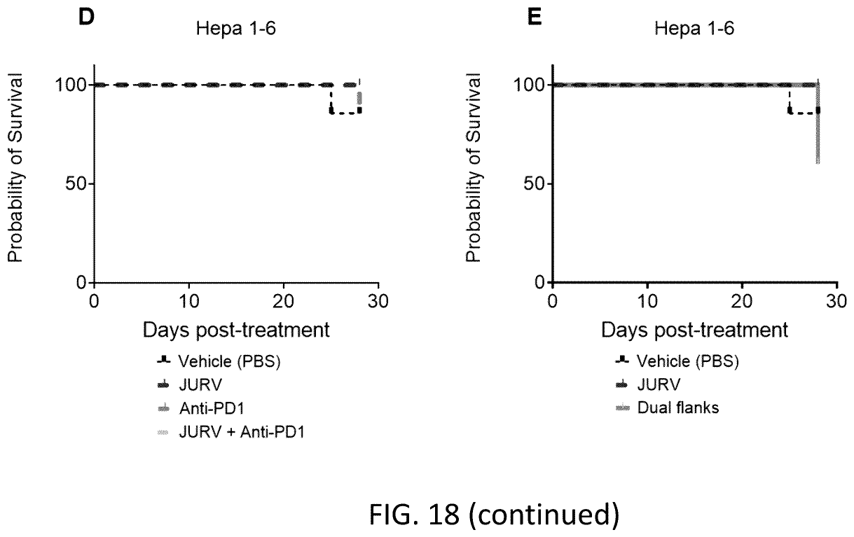

We first employed a subcutaneous syngeneic HCC model to assess the efficacy of JURV when combined with anti-PD-1 antibodies compared with anti-PD-1 alone or JURV alone. Treatment efficacy was evaluated in Hepa 1-6 tumors grafted into the right flanks of immunocompetent mice. An extended dosing approach, including intratumorally (IT) doses of JURV (3 doses) for groups treated with JURV and intraperitoneal doses of anti-PD-1 (6 doses) for immune checkpoint blockage groups (FIG. 17B). A significant (p<0.0001) tumor growth delay was observed in the JURV-treated mice compared to the control group (PBS) by day 28 post-treatment (FIG. 17A). The antitumor effect of anti-PD-1 therapy was comparable (p<0.0001) to that of JURV (FIG. 17A). However, we found that the tumor growth delay in mice treated with JURV combined with anti-PD-1 antibodies was less striking (p<0.001) than that of JURV alone or anti-PD-1 alone. Further analysis of the individual tumor volumes of mice indicates that nearly all (>70%) mice in the JURV-treated and anti-PD-1-treated groups eliminated their tumors compared to JURV combined with anti-PD-1 antibodies (>50%) groups (FIG. 18A).

We next sought to investigate whether local administration of JURV could elicit a systemic anti-tumor effect that could impact both local and distant tumor developments. We subcutaneously implanted HEPA 1-6 tumors on the right and left flanks of immunocompetent mice; however, we performed local IT injections of JURV on the right flanks only, leaving the left flanks unaffected. Our data indicate that IT injections of JURV triggered an anti-tumor activity that reduced the growth of HEPA 1-6 tumors on both right and left flanks (FIG. 17C, FIG. 18B). Moreover, starting on day 18, we observed a drastic drop in tumor volume on left flank tumors of mice that had not been inoculated with the virus. These results align with the induction of systemic anti-tumor activity commonly observed with oncolytic viruses in clinical trials and animal models.^{3,37} We also noted a slight drop in mice weight a day post-administration of the last dose of JURV; however mice quickly recovered, and no adverse events were noted until the end of the study (day 28) (FIG. 18C). In addition, one mouse was found dead in the control (PBS) group, but there was no significant difference in survival between the different groups (FIGS. 18D and E). Assessment of the level of biomarkers in the serum that are associated with viral treatment-related liver and kidney toxicity indicated mice did not experience severe virus-induced toxicity. These primary investigations highlight the potential of JURV in the induction of antitumor activity against local and distant non-injected tumors while not causing harmful effects in normal tissue, which is critical for targeting oligometastatic or metastatic diseases.

### In Vivo Antitumor Efficacy of JURV Across Various Murine Solid Tumor Models

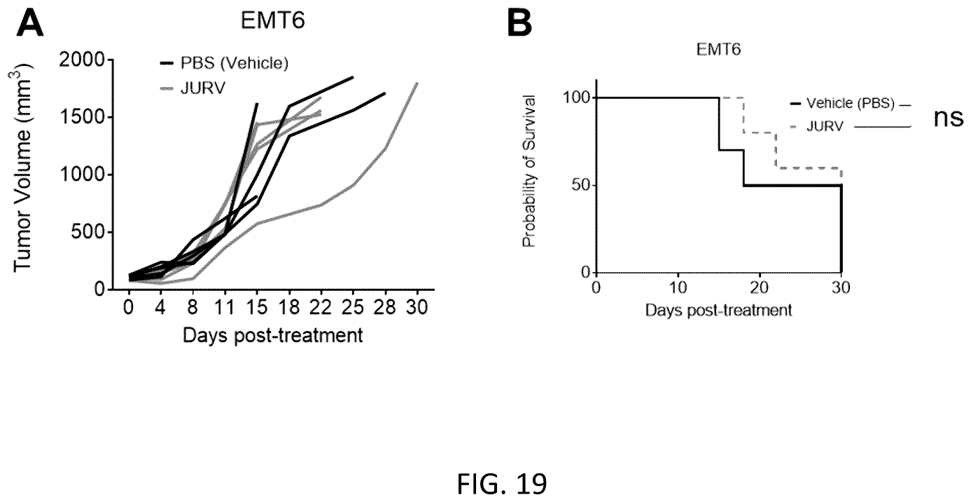

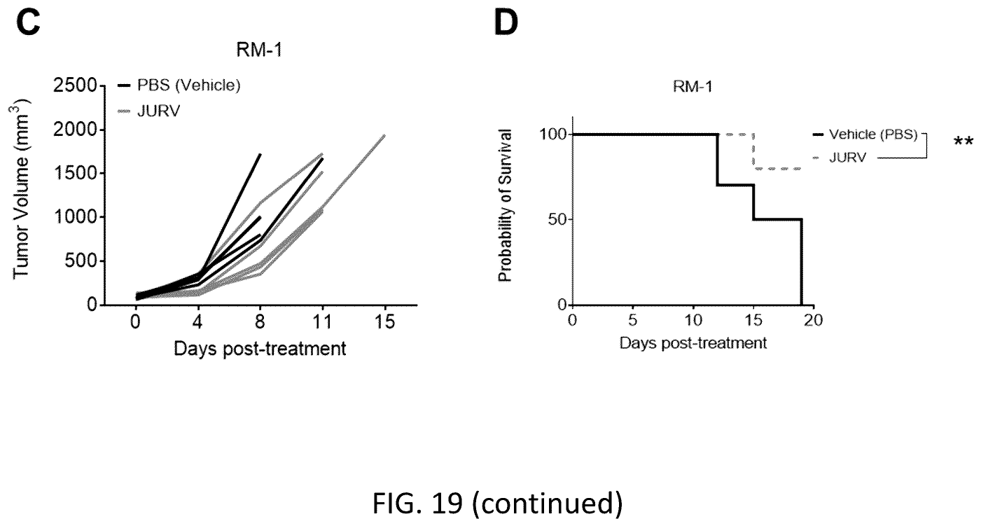

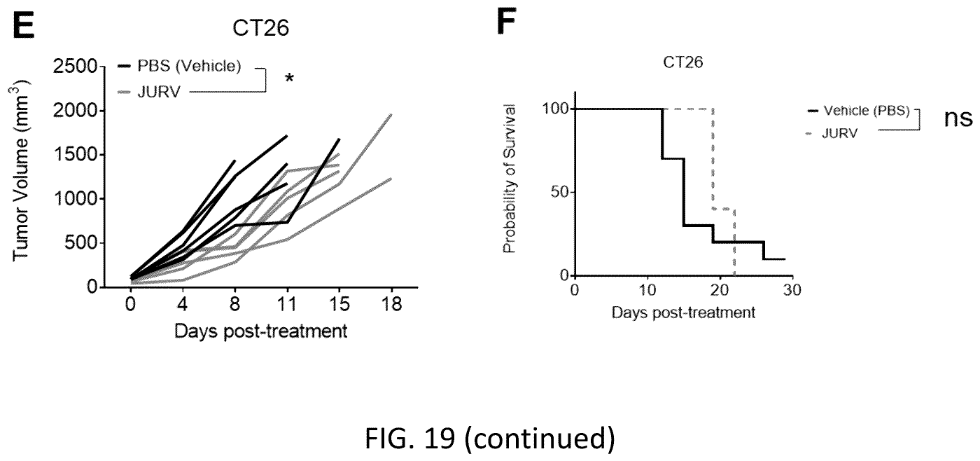

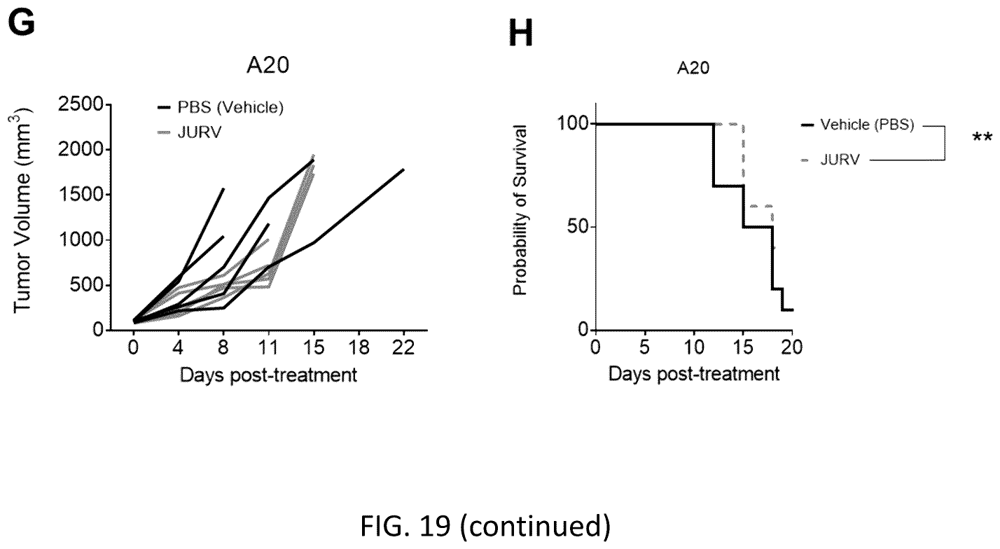

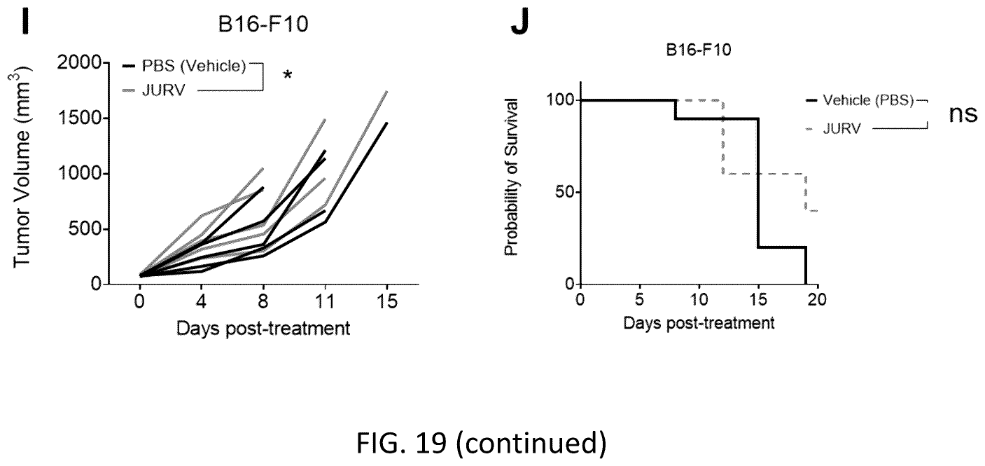

To assess the effectiveness of JURV-based therapy in other solid tumor models, we implanted several murine tumor cells in immune competent mice. These models include breast cancer (EMT6; FIG. 19A-B), colon cancer (CT26; FIG. 19E-F), reticulum sarcoma (A20; FIG. 19G-H), breast cancer (B16-F10; FIG. 19I-J), and prostate cancer (RM-1; FIG. 19C-D). Once tumor reached a treatable size as described in the methods section, mice were administered IT with a single injection of 50p of PBS or 1×10⁷ TCID₅₀ of JURV (FIG. 19). We found a heterogeneity in response to JURV in these models. Our data showed that JURV induced a significant tumor growth delay in B16-F10 (p=0.02) and CT26 (p=0.03), but not in A20, EMT6 and RM-1 models (FIGS. 19E and I). Interestingly, JURV provided survival benefits RM-1 model only (FIG. 19D). However, due to the aggressiveness of these syngeneic tumor models, a significant number of animals in the study reached tumor burden and had to be sacrificed before the end of the study. Overall, treatment was well tolerated in mice, and drug-related toxicity was absent in the study. These data demonstrate that JURV exerts anti-tumor activity across multiple tumor types; however, its variable activity suggest that JURV combination therapy with other anti-cancer drugs may yield long-lasting therapeutic effect.

JURV Combined with PD-1 Blockade Profoundly Modulates the Immune Component of the Tumor Microenvironment in Murine HCC

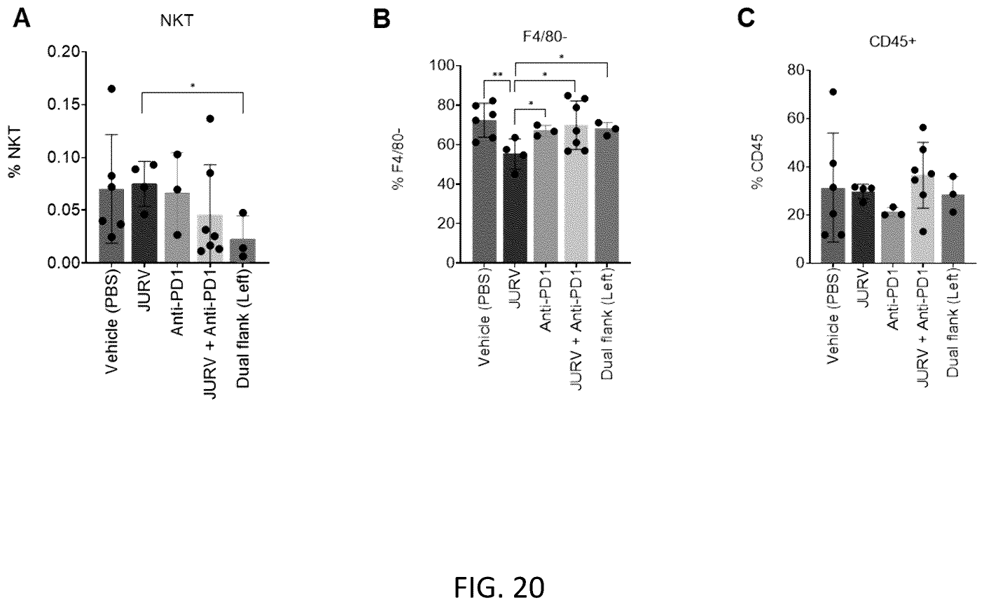

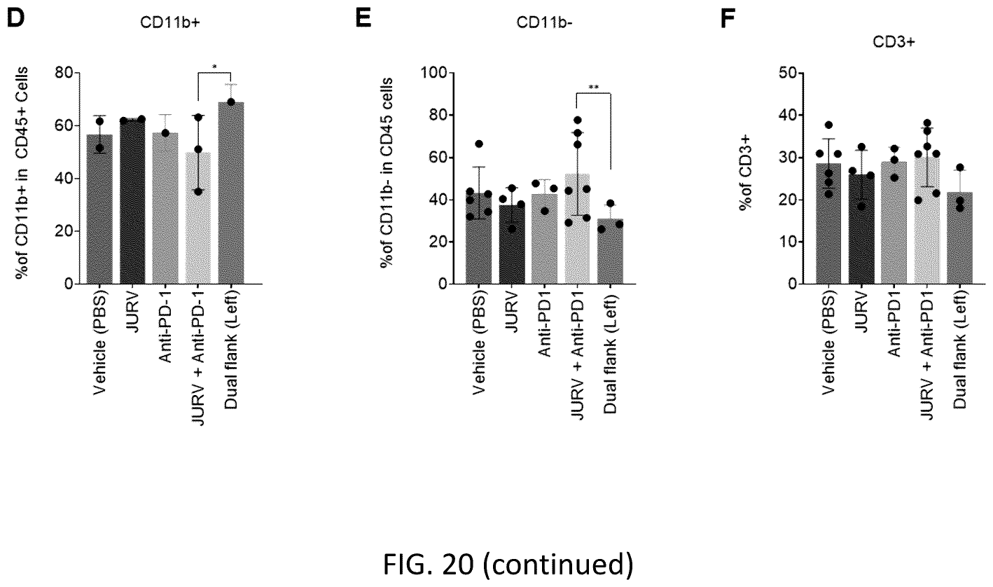

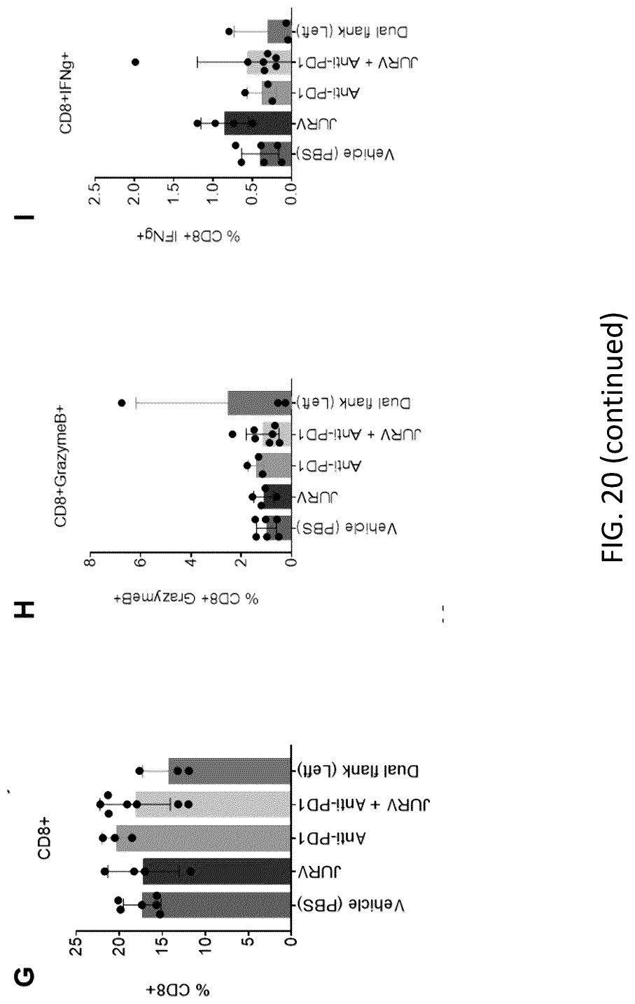

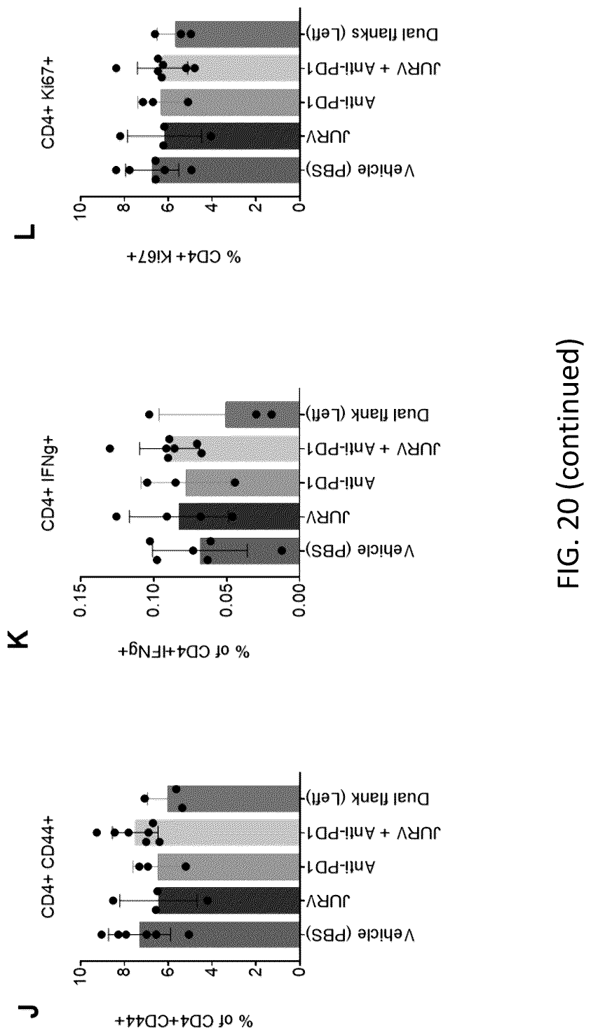

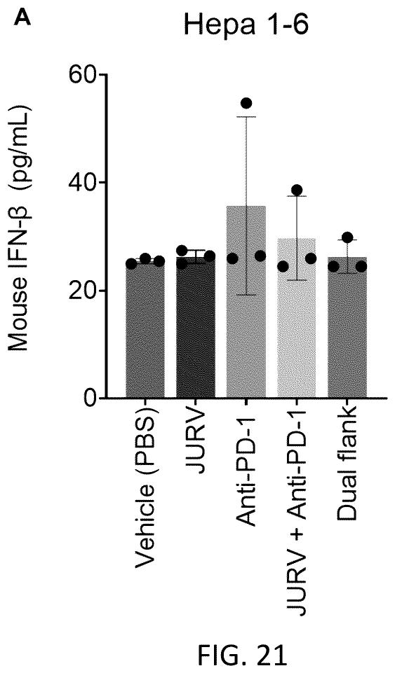

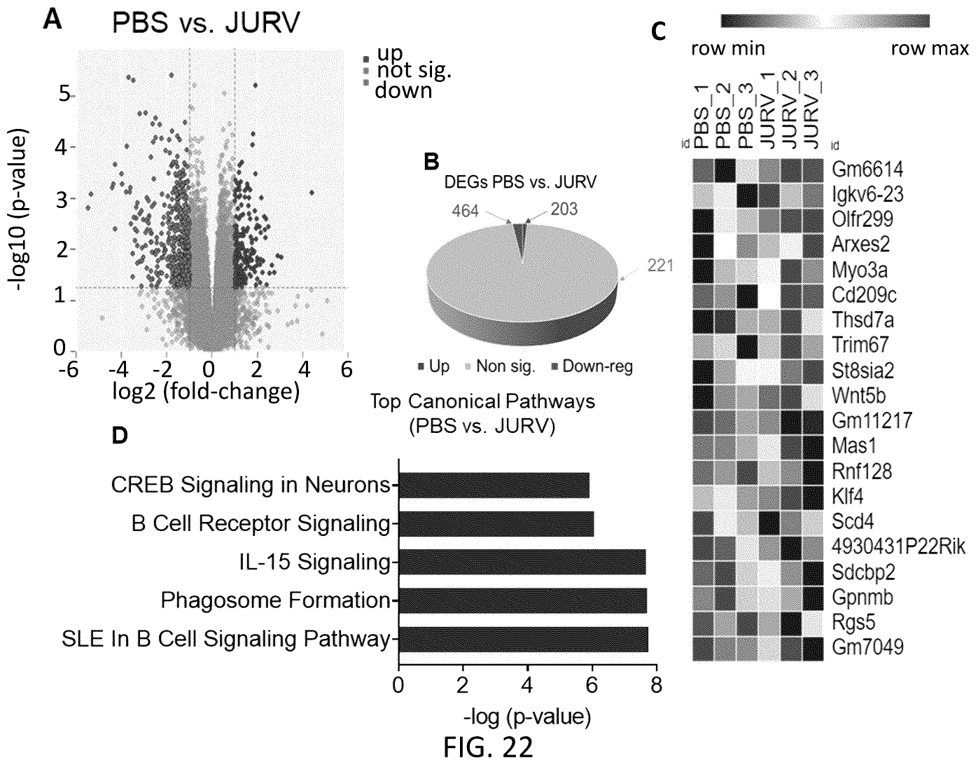

Many studies have shown that vesiculoviruses selectively infect, replicate, and lyse tumor cells and modulate local and systemic anti-tumor immune responses.³⁸ Given that administration of JURV, anti-PD-1 antibodies or a combination of JURV and anti-PD-1 antibodies evoked prominent tumor growth delay in the subcutaneous syngeneic HCC model, we set out to investigate and compare the changes in the immune responses associated with these different treatment regimens. As expected, our data show dramatic changes in the frequencies of tumor-infiltrating lymphocytes (TILs) after administering these therapies to mice. Intratumoral administration of JURV significantly decreased the subset of (p<0.001) F4/80− TILs (FIG. 20B) and increased the distribution of (p<0.01) M2-like macrophages (FIG. 17K), (p<0.01) natural killer (NK) cells (FIG. 17L), and (p<0.01) natural killer T (NKT) cells (FIG. 20A) compared to control (PBS), anti-PD-1, and dual flank (left), indicating virus replication and phagocytosis of infected tumor cells.³⁹ Treatments with anti-PD-1 antibodies correlated with the intratumoral accumulation of cytotoxic CD8+ T cells, mainly (p<0.01) CD8+ Ki67+ (FIG. 17D), (p<0.01) CD8+ PD-1+ (FIG. 17E), and p<0.001) CD8+ CD44+ (FIG. 17F), compared to PBS, JURV, and dual flank (left). We also noted a prominent decrease in the TILs macrophages (p<0.001) (anti-PD-1 vs. PBS) (FIG. 17I), (p<0.01) MI (anti-PD-1 vs. PBS) (FIG. 17J), and (p<0.01) M2-like (anti-PD-1 vs. JURV) (FIG. 17K). M2-like macrophages, which along with myeloid cells, are known to disable anti-tumor immunity via expression and interaction of PD-L1 with PD-1 on the surface of cytotoxic T cells.^{40,41} These results suggest that responding CTLs in the anti-PD-1 group may be tumor specific and contain effector memory cells in in tumor 0.42 Furthermore, we found that combining JURV and anti-PD-1 antibodies profoundly modulate the TME as manifested by an increase in (p<0.001) CD4+(FIG. 17G), (p<0.01) F4/80− (FIG. 20B), (p<0.01) CD4+ PD-1+ (FIG. 17H), (p<0.01) CD11b+ (FIG. 20D), (p<0.001) CD11b− (FIG. 20E), and decrease in (p<0.001) CD8+PD-1+ (FIG. 17E), (p<0.001) NK (FIG. 17L) compared to JURV, anti-PD-1, and dual flank (left). No significant change was observed in serum IFN-β in mice treated with JURV, anti-PD-1, and JURV combined with anti-PD-1 antibodies and dual flanks (FIG. 22). Collectively, our data suggest that the combination of JURV and anti-PD-1 antibodies elicit anti-tumor immunity via recruitment and tumor infiltration of cytotoxic effector T (CTLs) cells,^{43,44} inhibiting immunosuppression which is the hallmark of durable response to immunotherapy.

### Multi-Omics Analysis Identified Key Molecular Mechanisms of the Anti-Tumor Activity of JURV In Vivo

To determine the impact of IT administration of JURV on gene expression profiles in the tumor, we examined the transcriptome of murine HCC tumors injected with three doses of JURV. Differentially expressed genes (DEGs) were analyzed using the limma-voom method.⁴⁵ Our data (FIGS. 22A and B) showed that among the 22,786 genes, 203 DEGs were up-regulated and 463 DEGs were downregulated (2-fold change >2, p-value <0.055). Several of the 10 up-regulated DEGs Myo3a⁴⁶, Cd209c⁴⁷, Trim67⁴⁸, St8sia2⁴⁹, and Wnt5b⁵⁰ are associated with immune response pathways (FIG. 22C). Many of the enriched cellular signaling pathways, such as the B Cell Receptor Signaling, IL-15 Signaling, and Phagosome formation, identified by IPA analysis are related to the activation of the host's innate and adaptive immune responses (FIG. 22D).

Furthermore, to better understand the mechanism of JURV-induced anti-tumor activity, we analyzed the DEPs and DEGs from the transcriptomic and proteomic data. In the associated DEGs/DEPs, we identified the top 30 enriched features that are significantly upregulated or downregulated in the JURV group compared to the control group (PBS). Among the upregulated features, the S1pr3⁵¹, Tnpol⁵², Psmb10⁵¹, Ddt⁵⁴, Ncor2⁵⁵, S1c04c1⁵⁶ have been identified in inflammation, host immune response against microorganisms (virus, bacteria) and tumorigenesis.

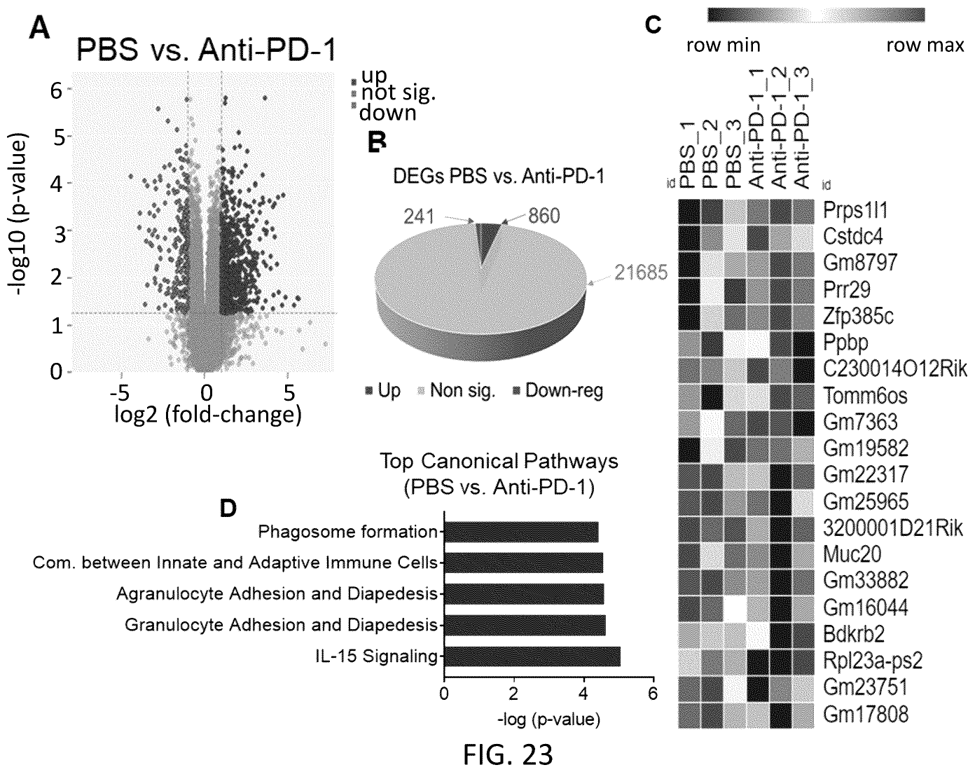

Similarly, our data show that IP administrations of anti-PD-1 antibodies are accompanied by significant transcriptional and proteomic changes in the TME, promoting anti-tumor immune reactions. Indeed, we found that out of 22,786 genes, 860 DEGS were up-regulated, and 241 DEGs were down-regulated in the HEPA 1-6 tumors (FIGS. 23A and B). All the top 10 DEGs (FIG. 23C), including Prps1l1⁵⁷, Cstdc4⁵⁸, and Ppbp⁵⁹ that are upregulated in the anti-PD-1 therapy group compared to control (PBS), are involved in inflammation, adaptive immune response, and cell survival and proliferation. Most enriched pathways (FIG. 23D) predicted by IPA analysis (i.e., Phagosome, communication between Innate and Adaptive Immune Cells) are immune-related pathways. Integrated analysis of the associated DEPs/DEGs features revealed upregulation of Tmod3⁶⁰, Nfkb2⁶¹, Dapk3⁶², Nipsnap3b⁶³, Sart3⁶⁴, and Adgrl4⁶⁵ which are key effectors of pathways regulating immunosuppression, angiogenesis, inflammation and anti-tumor immunity. These studies reveal potential molecular mechanisms involved in the JTRV- and/or anti-PD1-induced anti-tumor activities.

Multi-Omics Analysis Predicts that Combining JURV with Anti-PD-1 Antibodies Effectively Activates Anti-Tumor Immunity

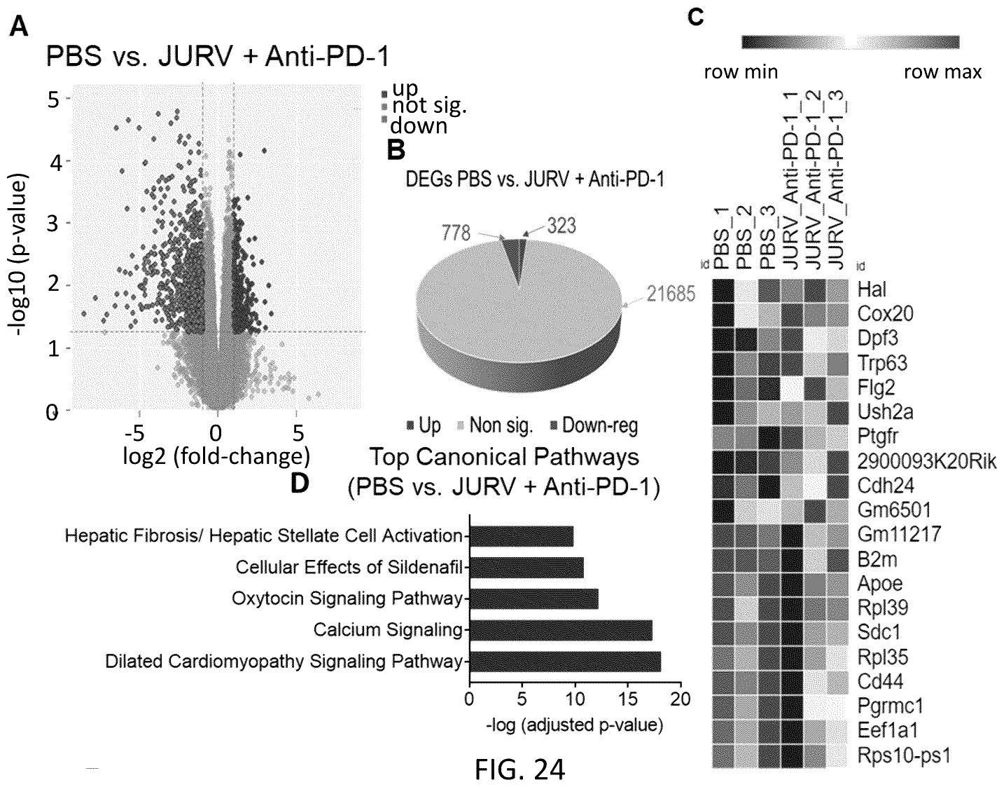

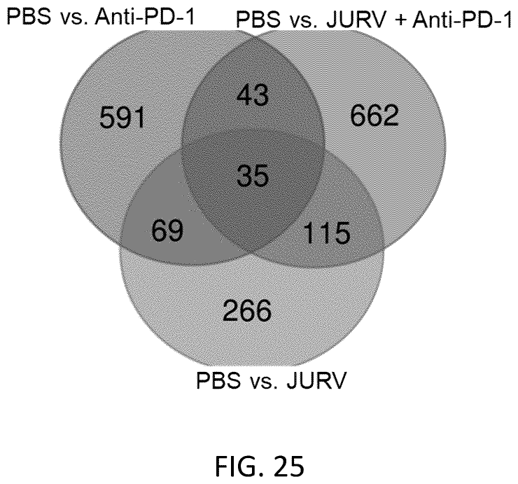

We analyzed the transcriptional profile of murine HCC to identify genes and pathways dysregulated in tumor following treatment with JURV, anti-PD-1 antibodies, and combination JURV and anti-PD-1 antibodies as compared to control (PBS). We analyzed 22,786 genes between the control group (PBS) and combination JURV and anti-PD-1 antibodies, and we found that 323 DEGs were up-regulated, whereas 778 DEGs were downregulated (FIGS. 24A and B). The top 10 up regulated DEGs, including the Cox20⁶⁶, Dpf3⁶⁷, Trp63⁶⁸, Flg2⁶⁹, Ush2a⁷⁰, and Cdh24⁷¹, are effector genes associated with tumor suppression and immune cells infiltration mechanism (FIG. 24C). IPA analysis identified enriched pathways (i.e., Hepatic Fibrosis/Hepatic Stellate Cell Activation, Oxytocin Signaling, Calcium Signaling pathways) which are all primarily associated with activation, and regulation of immune cells (FIG. 24D). Integrated analysis of the associated DEPs/DEGs, highlighted the up-regulation of genes linked to susceptibility to immunotherapy, autophagy, tumor repression and immune responses such as Cdk5rl⁷², Ptgdr2⁷³, Crip2⁷¹, Tardnp⁷⁵ Furthermore, examination of unique and common DEGs between PBS vs. JURV, PBS vs. Anti-PD-1, and PBS vs. JURV+Anti-PD-1 (FIG. 25) uncovered a relatively small overlap between co-differentially expressed genes (both up-regulated and down-regulated) among these three groups, suggesting the existence of differences in the mechanism of anti-tumor responses to these therapies, potentially driven by differences in the type of TILs these two treatment modalities activate, recruit and traffic to the tumor site or between the two routes of delivery (IP and IT).

Moreover, we performed gene ontology (GO) term enrichment analysis on 35 up-regulated or down-regulated DEGs (Table 3) common to all three data sets. Some mitogen-activated protein kinase (MAPK)⁷⁶ pathways known to be up-regulated during virus infection, macrophage migration inhibitory factor (MIF) 77, involved in inflammatory and immune response, necroptosis signaling, and vascular endothelial growth factor (VEGF) family were found to be enriched. These data indicate that even though JURV and anti-PD-1 therapies display different mechanisms of anti-tumor activity, they activate important and complementary pathways involved in innate and adaptive anti-tumor immunity, resulting in tumor growth control and regression.

TABLE 3
DEGs between PBS vs. JURV vs. PBS vs. Anti-PD-1
vs. PBS vs. JURV + Anti-PD-1 in murine HCC.

| | | GenBank/Gene | |
|---|---|---|---|
| Symbol | Entrez Gene Name | Symbol | Location |
| ABCA12 | ATP binding cassette subfamily A member 12 | Abca12 | Plasma Membrane |
| AI463229 | expressed sequence AI463229 | AI463229 | Other |
| BCAS1 | brain enriched myelin associated protein 1 | Bcas1 | Plasma Membrane |
| CLVS1 | clavesin 1 | Clvs1 | Cytoplasm |
| CNTN5 | contactin 5 | Cntn5 | Plasma Membrane |
| COLEC11 | collectin subfamily member 11 | Colec11 | Extracellular Space |
| CPS1 | carbamoyl-phosphate synthase 1 | Cps1 | Cytoplasm |
| CSTA | cystatin A | Cstdc4 | Cytoplasm |
| DNAH12 | dynein axonemal heavy chain 12 | Dnah12 | Cytoplasm |
| GDF1 | growth differentiation factor 1 | Gdf1 | Extracellular Space |
| Gm16202 | sorting nexin 4 pseudogene | Gm16202 | Other |
| Gm19582 | predicted gene, 19582 | Gm19582 | Other |
| Gm22317 | — | Gm22317 | Other |
| Gm38403 | predicted gene, 38403 | Gm38403 | Other |
| Gm7001 | CDK5 regulatory subunit associated protein 3 | Gm7001 | Other |
| | pseudogene | | |
| INSM1 | INSM transcriptional repressor 1 | Insm1 | Nucleus |
| LRRN4 | leucine rich repeat neuronal 4 | Lrrn4 | Plasma Membrane |
| LRRTM4 | leucine rich repeat transmembrane neuronal 4 | Lrrtm4 | Extracellular Space |
| MACROD2 | mono-ADP ribosylhydrolase 2 | Macrod2 | Nucleus |
| mir-679 | microRNA 679 | Mir679 | Cytoplasm |
| MYO3A | myosin IIIA | Myo3a | Cytoplasm |
| NALCN | sodium leak channel, non-selective | Nalcn | Plasma Membrane |
| NTM | neurotrimin | Ntm | Plasma Membrane |
| PABPC1L | poly(A) binding protein cytoplasmic 1 like | Pabpc1l | Cytoplasm |
| PLA2G2F | phospholipase A2 group IIF | Pla2g2f | Extracellular Space |
| PRELID3A | PRELI domain containing 3A | Prelid3a | Cytoplasm |
| ROBO2 | roundabout guidance receptor 2 | Robo2 | Plasma Membrane |
| Slc25a2 | solute carrier family 25 (mitochondrial carrier, | Slc25a2 | Cytoplasm |
| | ornithine transporter) member 2 | | |
| SMAD9 | SMAD family member 9 | Smad9 | Nucleus |
| TMPRSS3 | transmembrane serine protease 3 | Tmprss3 | Plasma Membrane |
| TOGARAM2 | TOG array regulator of axonemal microtubules | Togaram2 | Other |
| | 2 | | |
| Trbv3 | T cell receptor beta, variable 3 | Trbv3 | Other |
| TRIM67 | tripartite motif containing 67 | Trim67 | Cytoplasm |
| TTLL11 | tubulin tyrosine ligase like 11 | Ttll11 | Cytoplasm |
| UCP3 | uncoupling protein 3 | Ucp3 | Cytoplasm |

JURV Induces Anti-Tumor Immunity that Targets Both Local and Distant Tumors

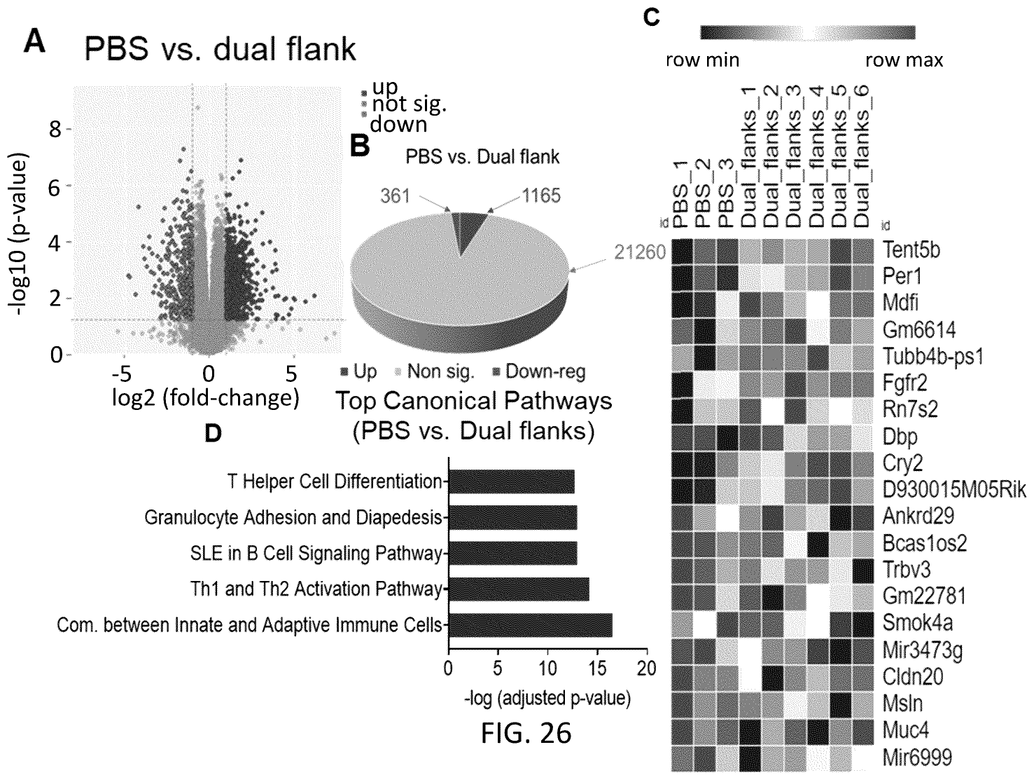

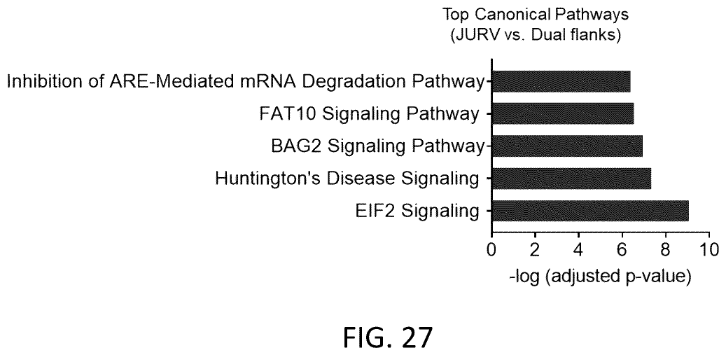

Previous reports have demonstrated that oncolytic viruses can induce virus-mediated activation of local and systemic anti-tumor immunity through the recruitment of class I MHC-restricted virus-specific and tumor-specific CTLs.^{78,79} To better comprehend the mechanisms behind the observed abscopal effect of JURV on distant tumors, we analyzed the transcriptome and proteome of non-injected (left flank) HEPA 1-6 tumors. Among 21,260 analyzed genes, we have found that 1165 DEGs were up-regulated and 361 DEGs were down-regulated in the treated group compared with PBS control groups (FIGS. 26A and B). Top ten up-regulated genes are listed in Table 4. Many top up-regulated genes, including Tent5b⁸⁰, Per1^{81,82}, Tubb4b-ps1⁸³, Dbp^{84,85}, and Cry2^{85,86}, play important tumor repressor activities in mammalian cells (FIG. 26C). We also found that Fgfr2 was up-regulated in our dataset. On the one hand, overexpression of Fgfr2 and its fusion partners have been linked to several advanced cancers, including HCC liver bile duct cancers, making the Fgfr2 pathway an attractive target in liver cancer.⁸⁷⁻⁹³ Conversely, studies have shown that treatment with pazopanib, multi-targeted receptor tyrosine kinase inhibitor, produced better outcomes in gastric tumors with Fgfr2 amplification.⁹⁰ The exact role of Fgfr2 in response to immunotherapy has yet to be determined and is worth future investigations, especially in virotherapy. Cry2 is associated with better prognosis in ERC/HER2− tumors.^{85,86} The efficacy of JURV in treating ERC/HER2− tumors is also worth investigating in the future. To the best of our knowledge, the biological function of the Gm6614, Rn7s2, and D930015M05Rik genes have not been defined yet (Table 4). IPA analysis allowed the identification of the most enriched canonical pathways in this dataset (FIG. 26D). These enriched biological pathways comprise the T helper cell differentiation, granulocyte adhesion, diapedesis, B cell signaling, Th1 and Th2 activation signaling, as well as communication signaling between innate and adaptive immune cells, indicating that modulation of the T helper cell pathways⁹⁴ played a crucial role in the abscopal effect induced by the IT injection of JURV (FIG. 26D). A comparison of DEPs/DEGs further confirmed that most up-regulated features, including Anxa3⁹⁵, Hspg2⁹⁶, Cyp2e1⁹⁷ and Map1lc3a⁹⁸ are therapeutic targets in immunotherapy or function as tumor suppressor genes. In addition, to assess the relationship between the local and systemic anti-tumor immunity in the murine tumors, we performed GO term enrichment analysis of DEGs between the JURV-injected and non-injected HEPA 1-6 tumors. The top enriched canonical pathways (i.e., Inhibition of ARE-Mediated mRNA Degradation, FAT10 Signaling, EILF2 Signaling) identified by analysis of DEGs between JURV vs. Dual flank are linked to response to immunotherapy, activation of MAPK pathways, and apoptosis (FIG. 27). These results indicate that JURV can effectively prime anti-tumor immunity against local and distant tumors in the studied HCC model.

TABLE 4
List of DEGs up and down-regulated in the dual flanks.

| Genes | Description of genes |
|---|---|
| Tent5b | Overexpression of Tent5b inhibited tumor cell cycle progression and cell |
| | proliferation in vitro and in vivo |
| Per1 | Per1 acts as a transcriptional repressor of clock-dependent genes to |
| | establishing a negative transcriptional feedback circuit controlling |
| | circadian periodicity |
| Mdfi | MyoD family inhibitor. Depending on the binding partners, Mdfi may inhibit |
| | or promotor tumor growth |
| Gm6614 | Predicted to enable bile acid transmembrane transporter activity and |
| | sodium-independent organic anion transmembrane transporter activity. |
| Tubb4b-ps1 | Function in stabilizing microtubule polymerization to prevent tumor |
| | metastasis |
| Fgfr2 | Abnormalities in Fgfr2 may contribute to carcinogenesis. |
| | The treatment of cancers with FGFR2 amplification with pazopanib |
| | resulted in a significant decrease in cell survival, while the same |
| | treatment showed no growth inhibitory effect on cancers without FGFR2 |
| | amplification |
| Rn7s2 | Rn7s2 may function in RNA processing, mRNAs nucleo-cytoplasmic |
| | transporting, or translational control. |
| Dbp | Play a role in many cellular processes including response to nucleolar |
| | stress, tumor suppression, and synthesis of ribosomal DNA. |
| Cry2 | A key component of the molecular clockwork, high expression of Cry2 |
| | and other clockwork components was reported to associate |
| | with longer metastasis-free survival. |
| | Cry2 may associate with better prognosis in ERC/HER2− tumors. |
| D930015M05Rik | No function about this was reported |

### It Administration of JURV Mediates Robust Anti-Tumor Efficacy HEP3B-Xenograft Models of HCC

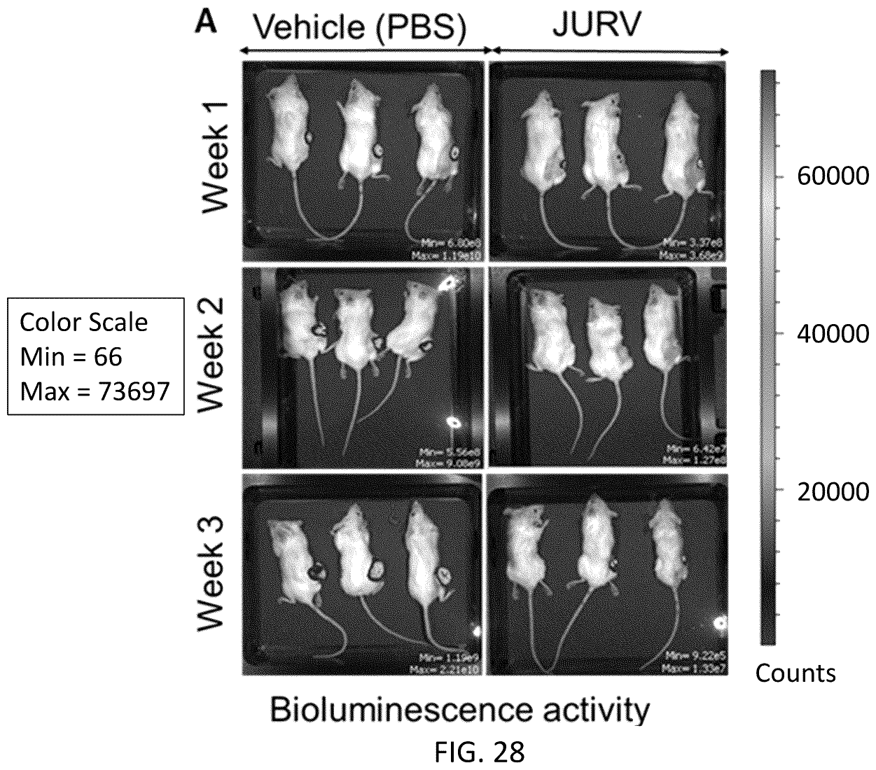

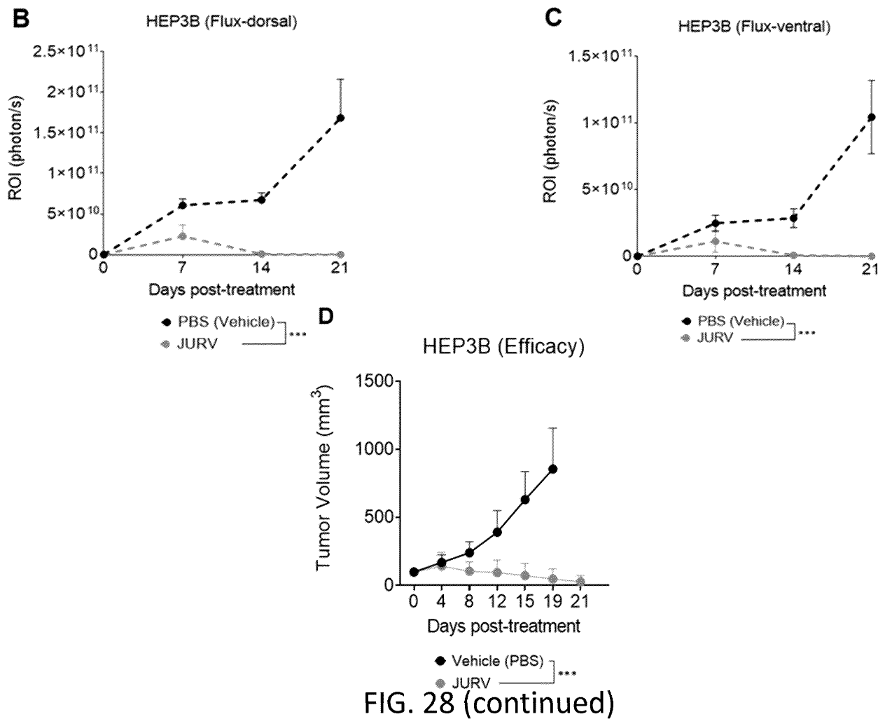

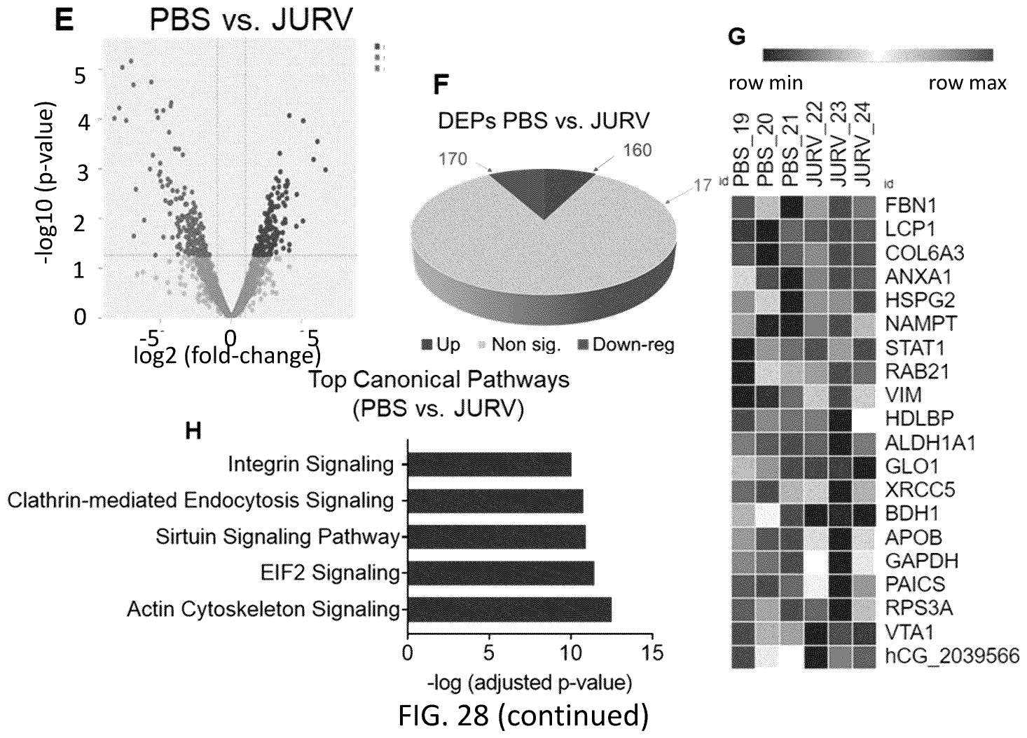

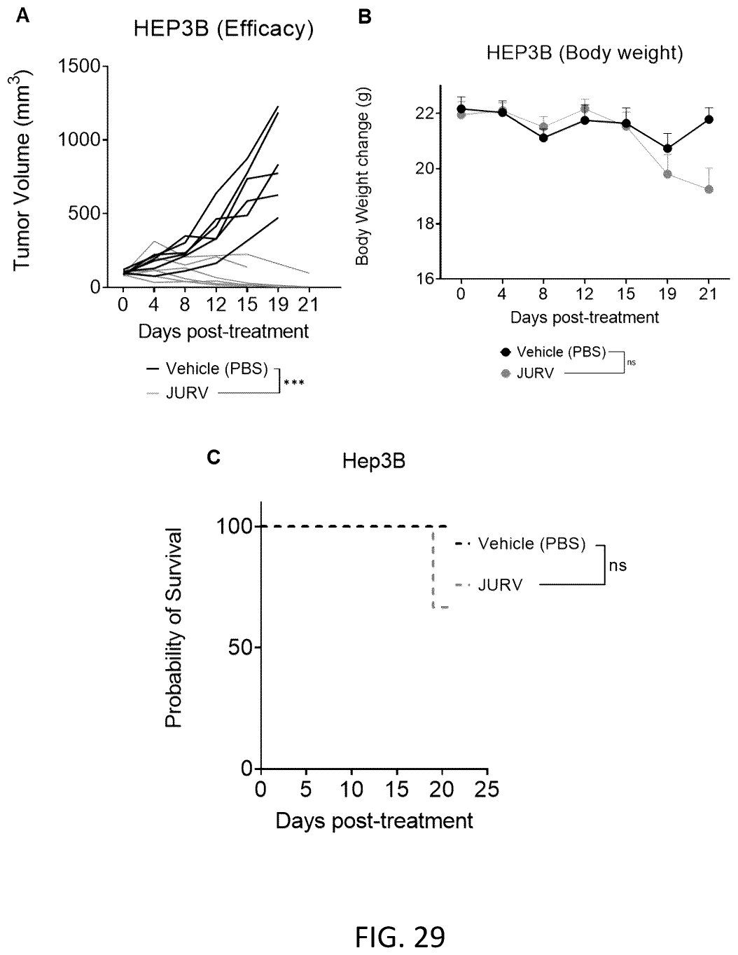

We have previously shown that responsiveness to Type I IFN production or viral kinetic in vitro by infected cancer cell lines does not always correlate with in vivo efficacy of oncolytic.⁷ To determine whether JURV can induce an oncolysis-dependent tumor cell killing in vivo, we injected IT three doses of JURV into HEP3B-xenografts. We employed luciferase tagged HEP3B cells to monitor tumor growth during the first three weeks of treatment. Compared to the control group (PBS), bioluminescence imaging showed a remarkable (p<0.0001) tumor inhibition (FIG. 28A-C) in the JURV-treated mice, which was visible from the first-week post-injection to the end of the study. In addition to luciferase activity, a comparison of tumor volumes between PBS vs. JURV indicates that JURV triggered a significant (p<0.0001) tumor growth delay (FIG. 28D, FIG. 29A). We next performed proteomics analysis of tumor tissues to determine the changes occurring after IT delivery of JURV in HEP3B tumors. Analysis of a total of 2,088 proteins showed a storm of 860 DEPs were up-regulated proteins and 241 DEPs were down-regulated in the JURV vs. PBS tumors (FIGS. 28E and F). Out of the 10 top up-regulated DEPs, we identified LCP1⁹⁹, COL6A3¹⁰⁰, HSPG2¹⁰¹, NAMPT¹⁰², STAT1¹⁰³ and VIM¹⁰⁴ as proteins associated with activation of the mTORC2/AKT pathways, prognostic and treatment of cancer, regulation of tumor growth, and cancer cell stiffness. There was a drop (˜15%) in the body weight in the JURV group starting on day 19 (FIG. 29B). However, one mouse was found moribund on day 18, which we attributed to an isolated adverse event due to the severe immunodeficient nature¹⁰⁵ of the NOD-SCID mouse model as no other mice experienced toxicity (FIG. 29C). Our findings show that JURV efficiently infects and lyses human HCC cells in vivo in human HEP3B-xenografts, which further increases enthusiasm around using the new immunovirotherapy in human HCC.

### Discussion

Rhabdoviruses possess several advantageous properties over other oncolytic viral vector platforms, including their amenability to genetic manipulation, and not using humans as a natural host that resulted in low seroprevalence in the population.⁵ In addition to an episomal and fast kinetic cycle in tumor cells, most vesiculoviruses can encode large transgenes⁴ while maintaining the ability to infect, replicate and induce apoptosis in a vast array of cancer cells.

In recent decades, vesicular stomatitis viruses (VSV)-derived vectors, the prototype Rhabdoviridae, have advanced to different stages of clinical testing against various human cancers. Although multiple studies have confirmed the therapeutic efficacy of VSV-based vectors, barriers to FDA approval and clinical application remain. These obstacles include several reports of VSV-induced neurotoxicity, hepatotoxicity, and rapid clearance by the host immune system. Our previous studies have described the anti-tumor potential of MORV⁷, a non-VSV Rhabdoviridae with a natural tumor selectivity and hypersensitivity to type 1 interferon response which is defective in ¾ of all cancers.^{6,7} To expand the spectrum of the oncolytic virus backbone, in this study, we examined the potential anti-cancer role of Jurona virus (JURV), a genetically distinct member of the same virus family, as a therapeutic agent for cancers.

We evaluated the anti-tumor effects of JURV in human and murine hepatocellular carcinoma (HCC) cell lines in vitro and mouse models of HCC. JURV efficiently infected all the tested HCC cells with variability in cytolytic activity. Further investigation into the mechanism of the in vitro HCC-killing differences may provide insights into response to JURV-based therapy in HCC and potentially other solid tumors. Concerns of VSV-induced encephalitis and hepatotoxicity have led to the development of attenuated recombinant VSV platforms using different viral engineering techniques. Though these vectors showed improved safety to some extent, reduced oncolytic activity compared to the parental VSV was also reported due to impaired intratumoral replication capability.

In this study, we showed that naturally attenuated JURV displayed all the characteristics required for a potent immunovirotherapy, such as strong cytolytic effect and rapid replication cycle in tumor cells, sensitivity to type I IFN, and, more importantly, lack of long-term neurotoxicity and hepatotoxicity in mice. Analysis of the toxicoproteome of brain tissues indicated the activation of multiple mechanisms involved in pathogens clearance and neuron protection that limited viral spread and prevented brain damage. Moreover, our data demonstrated that JURV stimulates multiple mechanisms of innate and adaptive immune responses leading to tumor growth delay in a syngeneic HCC model. The combination of JURV and anti-PD-1 therapy considerably modulates the TME via enhanced infiltration of cytotoxic T-cells via activation and recruitment of distinct immune system effectors with complementary anti-tumor activities. Furthermore, we showed that JURV efficiently induces oncolysis-mediated tumor growth delay in human HCC xenografts. Analysis of mRNA and protein expression profiles in HCC tumors following administration of JURV, anti-PD-1 antibodies, and combination JURV and anti-PD-1 therapy unveiled the up-regulation of immune-related genes and identified enriched pathways involved in inflammation, regulation of immunosuppression, angiogenesis, and anti-tumor immunity. Interestingly, we found that IT administration of JURV was associated with an abscopal effect in a bilateral murine HCC. To elucidate the mechanisms contributing to the abscopal effect, we employed a proteomic analysis approach on untreated tumors implanted at a distant site of the animals. Our results indicate that JURV triggered the activation of several tumor-suppressive genes, suggesting that tumor-suppressive pathways may play a critical role in the abscopal effect in our animal model. In addition, our data indicate that cellular anti-tumor immunity via activation of the T helper cell pathways may also be a key player in the JURV-induced abscopal effect.

Together, our results demonstrate that JURV potently infect HCC cells and induce oncolysis in vitro and in animal model. It also indicates that JURV-infected tumor cells prime an anti-tumor immunity (targeting primary and distant tumors) which is enhanced by addition of anti-PD-1 antibodies.

### Methods

### Experimental Design

These experiments were performed to provide new and critical mechanistic insights into the safety and the efficacy oncolytic JURV in HCC tumor models, which will enable the rational design of studies using JURV as monotherapy or conjugated with other cancer therapies in early- or late-stage HCC for possibly additive or perhaps synergistic long-term responses in clinical settings. All animals were randomly allocated to the different study groups in an unblinded fashion. Average tumor volume (mm³) for each group+SEM at randomization was set between 80-120 mm³. The tumor volume (or its log transformation) was assessed in its relationship to time through a mixed linear regression model, using time, treatment effect, and their interaction as the independent variables. We used a random effect to account for within-subject correlation due to repeated measurements. Slopes were interpreted as growth rates (or logarithm) of the tumor over time and compared between groups. Analysis of Kaplan-Meier curves was used to identify the proportion of tumor-bearing mice living for a specific time after treatment. The n values and statistical methods are indicated in the statistical analysis section.

### Cell Lines

This study used a panel of three human hepatocellular carcinomas (HCC) cell lines (HEP3B, PLC, HuH7) and two murine HCC cell lines (HEPA 1-6, R1LWT). We also used several murine solid tumor cells, including breast carcinoma cells (EMT6), colon carcinoma cells (CT26), reticulum sarcoma cells (A20), skin melanoma cells (B16-F10), and prostate cancer cells (RM-1). All cell lines were cultured at 37° C. with 5% CO2 in media supplemented with antibiotic agents (100 μg ml-1 penicillin and 100 μg ml-1 streptomycin). HEP3B, PLC, and HuH7 were maintained in Dulbecco's Modified Eagle's Medium (DMEM) with 10% fetal bovine serum (FBS). We maintained HEPA 1-6, RILWT, BHK-21 (Baby Hamster kidney fibroblast), and Vero (African green monkey kidney) cells in DMEM with 10% fetal bovine serum (FBS). BHK-21 and Vero cells were obtained from the American Type Culture Collection (Manassas, VA). We purchased HEP3B, PLC, HuH7, HEPA 1-6, EMT6, A20, CT26, BF16-F10, and RM-1 cells from the American Type Culture Collection (ATCC, Manassas, VA). The RILWT cell line was a gift from Dr. Dan G. Duda at MGH, Boston, MA.

### Oncolytic Viruses

We obtained Jurona virus (JURV) from the University of Texas Medical Branch (UTMB) World Reference Center for Emerging Viruses and Arboviruses (WRCEVA). A laboratory-adapted viral clone of JURV was generated using sequential plaque purifications on Vero cells (ATCC, Manassas, VA). RNA-sequencing was applied to confirm the full-length JURV genome (10,993 bp) as previously described.⁴ Infectious JURV was recovered from a full-length cDNA clone (Genscript, USA) comprising genes encoding for the nucleoprotein (JURV-N), phosphoprotein (JURV-P), matrix protein (JURV-M), glycoprotein (JURV-G), and RNA-directed RNA polymerase L protein (JURV-L) as described by Lawson et al.¹⁴ Vesicular stomatitis virus (VSV) was rescued from the pXN2 cDNA plasmid, and virus stock was amplified on B-FK-21 cells. Sucrose density gradient centrifugation was used to obtain purified viral particles (VSV, MORV and recombinant JURV) before in vitro and in vivo studies.

### Amplification of Viral Stock

Viral amplification was done by infecting confluent (˜80%) Vero cells in T-175 flasks with a low multiplicity of infection (MOI) of 0.001 of JURV, MORV, or VSV. Forty-eight hours post-infection or when cytopathic effects (CPE) were observable. Supernatants of virus-infected cells were collected from the flasks. The viral stocks were purified using 10-40% sucrose-density gradient ultracentrifugation followed by dialysis. The titer (TCID₅₀) of each virus was determined by the Spearman-Karber algorithm using serial viral dilutions in BHK-21 cells.

### Cell Viability Assays

For all cytotoxicity assays (96-well format), 1.5×10⁴ cells were infected with JURV, MORV, or VSV at the indicated MOI of 10, 1, and 0.1 in serum-free Gibco Minimum Essential Media (Opti-MEM). Cell viability was determined using Cell Titer 96 AQueous One Solution Cell Proliferation Assay (Promega Corp, Madison, Wisconsin, USA). Data was generated as means of six replicates from two independent experiments+/−SEM.

### Crystal Violet Assays

Five hundred thousand cells were infected with oncolytic JURV in 6-well plates at an MOI of 0.1 for 1 h. Supernatants of virus-infected cells were removed, and cells were washed with PBS and incubated at 37° C. until analysis. At 72 hours after infection, cells were fixed with 5% glutaraldehyde and stained with 0.1% crystal violet to visualize cellular morphology and remaining adherence indicative of cell viability. Pictures of representative areas were taken.

### One-Step Viral Growth Kinetics

Two hundred thousand HCC cells were plated in each well of a 6-well plate in 2 mL of complete DMEM. After overnight rest, we infected the cells with JURV at an MOI of 0.1 for 1 hour. Supernatants of virus-infected cells were removed, and cells were washed with PBS, and fresh media was added. At timepoints 10, 24, 48, and 72 hours, the supernatant was collected and stored at −80 C. Viral titers (TCID₅₀) were determined with serial dilutions of the supernatant on Vero cells. Data was generated as means of two independent experiments+/−SEM.

### Interferon Sensitivity Assays

HCC cells were seeded in a 96-well plate at a density of 2.0×10⁴ cells/well and cultured overnight. Twenty-four hours post-infection, cells were pretreated with different concentrations of Universal type I IFN-α was added directly into the culture medium. After overnight incubation, fresh medium containing Universal Type I IFN-α (Catalog No. 11105-1; PBL Assay Science, USA) was added, and cells were infected with JURV at an MOI of 0.01. Cell viability was assessed using a Cell Titer 96 AQueous One Solution Cell Proliferation Assay (Promega Corp, Madison, Wisconsin, USA). Absorbance measurements at 490 nm were normalized to the maximum read per cell line, representing 100% viability. Data are shown from three independent experiments. For all cell viability experiments, absorbance was read using a Cytation 3 Plate Reader (BioTeK, Winooski, VT, USA). Data are expressed as means of triplicates from three independent experiments+/−SEM.

### Mice

Female C57BL6/J mice (Strain #:000664), BALB/cJ (Strain #:000651) mice, and NOD.Cg-Prkdc^{scid}/J mice (Strain #:001303) were purchased from Jackson Laboratories at 6-8 weeks of age. Male C57BL6/J mice (Strain #:000664) were also obtained from Jackson Laboratories. All mice were housed at the Division of Laboratory Animal Medicine (DLAM) at University of Arkansas for Medical Sciences (UAMS). The DL AM has a full staff of veterinarians and veterinary technicians who supervised and assisted in animal care throughout the studies. All animal studies were conducted in accordance with and approved by the Institutional Animal Care and Use Committee at the University of Arkansas for Medical Sciences.

### Analysis of Virus-Induced Adverse Events in Mice

Female C57BL/6J mice (N=6 mice/group) were intranasally (25 μL in each nostril) or intravenously (50 μL/mouse) administered with phosphate-buffered saline (PBS), moderately high dose (1×10⁷ TCID₅₀), or high dose (1×10⁸ TCID₅₀) of the virus. Body weight, temperature, behavior, and clinical signs were monitored by a board-certified veterinarian at least three times a week to detect any signs of toxicity. However, three days post-infection, three mice per group and tissues were collected (blood, brain, liver, and spleen) for short-term toxicity evaluation and viral biodistribution. The remaining mice were monitored for thirty days.

### Short-Term Toxicological Analysis of Blood Components

Blood was collected from the submandibular vein (cheek bleed) and cardiac puncture on day 3 post-treatment. Blood was collected in BD Microtainer tubes with ethylenediaminetetraacetic acid or lithium heparin (Becton, Dickinson and Company, Franklin Lakes, New Jersey, USA) for complete blood counts (CBC) or in BD Microtainer SST tubes (Becton, Dickinson, and Company) for serum analysis. CBC analysis was performed in an Abaxis Piccolo Xpress chemistry analyzer (Abaxis, Union City, California, USA), and blood chemistry analysis was done in a VetScan HM5 Hematology Analyzer (Abaxis).

### Toxicoproteomic Analysis

At three days post-inoculation of JURV, mouse brain and liver tissues were harvested and dehydrated using an increasing ethanol concentration and embedded into paraffin to become formalin-fixed paraffin-embedded (FFPE) blocks as previously described.¹²⁶ Tissue blocks were sectioned into 3-5 10 μm sections and underwent a deparaffinization procedure for FFPE tissue.¹²⁷ Following deparaffinization of FFPE samples with xylene and tissue lysis in sodium dodecyl sulfate, total protein was reduced, alkylated, and digested using filter-aided sample preparation¹²⁸ with sequencing grade modified porcine trypsin (Promega). Tryptic peptides were separated by reverse-phase XSelect CSH C18 2.5 μm resin (Waters) on an in-line 150×0.075 mm column using an UltiMate 3000 RSLCnano system (Thermo). Peptides were eluted using a 60 min gradient from 98:2 to 65:35 (buffer A, 0.1% formic acid, 0.5% acetonitrile: buffer B, 0.1% formic acid, 99.9% acetonitrile) ratio. Eluted peptides were ionized by electrospray (2.4 kV) followed by mass spectrometric (MS) analysis on an Orbitrap Exploris 480 mass spectrometer (Thermo). MS data were acquired using a Fourier transform MS (FTMS) analyzer in profile mode at a resolution of 120,000 over a range of 375 to 1500 m/z. Following HCD activation, MS/MS data were acquired using the FTMS analyzer in centroid mode at a resolution of 15,000 and normal mass range with normalized collision energy of 30%. Proteins were identified by database search using MaxQuant (Max Planck Institute) label-free quantification with a parent ion tolerance of 2.5 ppm and a fragment ion tolerance of 20 ppm. Scaffold Q+S (Proteome Software) was used to verify MS/MS-based peptide and protein identifications. Protein identifications were accepted if they could be established with less than 1% false discovery and contained at least two identified peptides. Protein probabilities were assigned by the Protein Prophet algorithm.¹²⁹

### In Vivo Efficacy of the Oncolytic JURV in a Syngeneic Mouse Model of HCC

To evaluate the in vivo therapeutic efficacy of oncolytic JURV in a syngeneic mouse HCC model, 1×10⁶ HEPA 1-6 cells in 100 μL of cold RPMI were injected subcutaneously into the right flanks of immunocompetent female C57BL6/J mice (n=7/group; Jackson Laboratory) using 1 mL syringes. Mice were monitored weekly for palpable tumors or any changes in appearance or behavior. When average tumors reached a treatable size (80-120 mm³), mice were randomized into the respective study groups and dosed within 24 hours of randomization. On days 0, 7, and 14, mice were administered 50 μL IT injections containing PBS (control group) or 1×10⁷ TCID₅₀ units of JURV (Test article group). Groups administered anti-PD-1 therapy or combination JURV+anti-PD-1 also receive 50 μL of anti-PD-1 antibodies intraperitoneally (IP) twice a week for three weeks. To establish the syngeneic bilateral (dual flanks) HCC tumors, HEPA 1-6 cells (1×10⁶ cells/mouse) were first subcutaneously grafted into the right flanks. These resulted in tumors in approximately 14 days and were categorized as “primary” tumors. Simultaneously, we performed distant HEPA 1-6 tumor grafts injection (1×10⁶ cells/mouse) into the left flanks of these mice. Mice in the dual flank group received 50 μL IT injections of 1×10⁷ TCID₅₀ units of JURV on their right flanks only once a week for three weeks. Tumor volume and body weight were measured twice weekly following randomization and initiation of treatment using a digital caliper and balance. Tumor volume was calculated using the following equation: (longest diameter*shortest diameter2)/2 with a digital caliper. During the first week of treatment and after each injection, mice were monitored daily for signs of recovery for up to 72 hours. Mice were euthanized when body weight loss exceeded 20%, when tumor size was larger than 2,000 m³ or for adverse effects of treatment. Mice were sacrificed 28 days following the first JURV dose administration, at which time tumor and blood were collected for downstream analysis.

### Hep3B Xenograft Model

Female NOD.Cg-Prkdc^{scid}/J mice were subcutaneously inoculated with HEP3B cells tagged with a firefly luciferase reporter gene on the right flanks (n=6/group). When the average tumor volume reached 80-120 mm³, mice were administered 50 μL IT injections of 1×10⁷ TCID₅₀ of JURV or 50 mL of PBS weekly for three weeks. Tumor volume was measured twice weekly until the end of the study (Day 21), or the humane endpoint as described above. We also recorded mouse body weight and clinical observations twice per week.

### Anti-Tumor Effect of JURV Across Multiple Solid Tumors

Female BALB/cJ mice were subcutaneously inoculated with EMT6 (breast cancer), CT26 (colon cancer), and A20 (reticulum sarcoma) cells on the right flanks (n=6-8/group). B16F10 melanoma cells were implanted into the right flanks of female C57BL6/J mice. RM-1 (prostate cancer) cells were grafted into the right flanks of male C57BL6/J mice. When the average tumor volume reached 80-120 mm³, mice (n=6-8/group) were administered a single 50 μL IT injections of 1×10⁷ TCID₅₀ of JURV or 50 μL of PBS. Tumor volume was measured twice weekly until the end of the study (Day 21), or the humane endpoint as described above. We also recorded mouse body weight and clinical observations twice per week.

### Bioluminescence Imaging

Tumor-bearing (HEP3B) mice were anesthetized with isoflurane and imaged once a week (days 0, 7, and 14) with the IVIS Xenogen imaging system to virus-induced changes in tumor growth. Anesthesia was induced in an induction chamber (2-5% isoflurane), after which the mice were placed in the imaging instrument and fitted with a nose cone connected to a vaporizer to maintain isoflurane (1.5-2%) during the procedure. This range of concentrations produces a level of anesthesia that prevents animal movement during scanning. If the respiratory rate accelerates or slows, the isoflurane concentration is increased or decreased. We used a heated animal bed, heating pads, and, if necessary, a heating lamp to ensure that body temperature is maintained both before imaging and during the procedure. Each mouse received an intraperitoneal injection of D-luciferin (Sigma-Aldrich #L9504; 150 mg/kg body weight in the volume of 10 μl/g of body weight, prepared in sterile water). Anesthetized mice were placed into the IVIS Xenogen imaging system on their stomachs. Imaging of each group of mice took less than 10 minutes. This is a non-invasive imaging procedure, and we needed no restraints.

### Analysis of Tumor-Infiltrating Immune Cells

Hepa 1-6 tumors (n=3 samples/group) were excised and dissociated using a mouse tumor dissociation kit (Miltenyi, CAT #130-096-730) with a gentleMACS™ Octo Dissociator (Miltenyi) according to the manufacturer's protocol. CD45⁺ cells were isolated with mouse CD45 (TIL) microbeads (Miltenyi). Cells were incubated with Fixable Viability Stain 510 for 15 minutes at 4° C. followed by anti-Fc blocking reagent (Biolegend, Cat #101320) for 10 minutes prior to surface staining. Cells were stained, followed by data acquisition with a BD LSRFortessa X-20 flow cytometer. All antibodies were used following the manufacturer's recommendation. Fluorescence Minus One control was used for each independent experiment to establish gating. For intracellular staining of granzyme B, cells were stained using an intracellular staining kit (Miltenyi), and analysis was performed using FlowJo™ (TreeStar). Forward scatter and side scatter cytometry were used to exclude cell debris and doublets.

### Flow Cytometry Antibody Analysis

The following antibodies were used for flow cytometry analysis: CD45-FITC (Cat. #553079; BD Biosciences), CD3-BUV395 (Cat. #563565; BD Biosciences), CD4-BUV737 (Cat. #612761; BD Biosciences), CD8-Percp-Cy5.5 (Cat. #45-0081-82; eBioscience), CD44-BV711 (Cat. #103057; Biolegend), CD335-PE/Dazzle594 (Cat. #137630; Biolegend), PD-1-PE (Cat. #551892; BD Biosciences), Ki67*-BV605 (Cat. #652413; Biolegend), Granzyme B*-APC (Cat. #366408; Biolegend), IFN-γ*-BV421 (Cat. #563376; BD Biosciences), CD11b-PE-Cy7 (Cat. #101216; Biolegend), F4/80-BV510 (Cat. #123135; Biolegend), CD206-AF700 (Cat. #141734; Biolegend), I-A/I-E-BV786 (Cat. #743875; BD Biosciences), and L/D-efluor780 (Cat. #65-0865-18; eBioscience). A full list of antibodies used can be found in Table 5.

TABLE 5
List of antibodies.

| Markers | Fluorochrome | Clone | Catalog | Isotypes | Vender |
|---|---|---|---|---|---|
| CD45 | FITC | 30-F11 | 553079 | Rat IgG2b, κ | BD |
| CD3 | BUV395 | 145-2C11 | 563565 | Armenian Hamster IgG1, κ | BD |
| CD4 | BUV737 | GK1.5 | 612761 | Rat IgG2b, κ | BD |
| CD8 | Percp-Cy5.5 | 53-6.7 | 45-0081-82 | Rat IgG2a, k | eBioscience |
| CD44 | BV711 | IM7 | 103057 | Rat IgG2b, k | Biolegend |
| CD335 | PE/DAzzle594 | 29A1.4 | 137630 | Rat IgG2a, κ | Biolegend |
| PD-1 | PE | J43 | 551892 | Armenian Hamster IgG2, κ | BD |
| Ki67* | BV605 | 16A8 | 652413 | Rat IgG2a, κ | Biolegend |
| Granzyme B* | APC | APC | QA18A28 | 396408 | Biolegend |
| IFN-γ* | BV421 | XMG1.2 | 563376 | Rat IgG1, κ | BD |
| CD11b | PE-Cy7 | M1/70 | 101216 | Rat IgG2b, κ | Biolegend |
| F4/80 | BV510 | BM8 | 123135 | Rat IgG2a, κ | Biolegend |
| CD206 | AF700 | C068C2 | 141734 | Rat IgG2a, κ | Biolegend |
| I-A/I-E | BV786 | 2G9 | 743875 | Rat IgG2a, κ | BD |
| L/D | efluo780 | NA | 65-0865-18 | NA | eBioscience |

### RNA-Sequencing of Murine HCC Tumors

Hepa 1-6 (n=3 samples/group) FFPE scrolls were processed for DNA and RNA extraction using a Quick-DNA/RNA FFPE Miniprep Kit with on-column DNase digestion for the RNA preps (Cat. #R1009; Zymo Research). RNA was assessed for mass concentration using the Qubit RNA Broad Range Assay Kit (Cat. #Q10211; Invitrogen) with a Qubit 4 fluorometer (Cat. #Q33238; Invitrogen). RNA quality was assessed with a Standard Sensitivity RNA Analysis Kit (Cat. #DNF-471-0500; Agilent) on a Fragment Analyzer System (Cat. #M5310AA; Agilent). Sequencing libraries were prepared using TruSeq Stranded Total RNA Library Prep Gold (Cat. #20020599; Illumina). RNA DV200 scores were used to determine fragmentation times. Libraries were assessed for mass concentration using a Qubit 1×dsDNA HS Assay Kit (Cat. #Q33231; Invitrogen) with a Qubit 4 fluorometer (Cat. #Q33238; Invitrogen). Library fragment size was assessed with a High Sensitivity NGS Fragment Analysis Kit (Cat. #DNF-474-0500; Agilent) on a Fragment Analyzer System (Cat. #M5310AA; Agilent). Libraries were functionally validated with a KAPA Universal Library Quantification Kit (Cat. #07960140001; Roche). Sequencing was performed to generate paired-end reads (2×100 bp) with a 200-cycle S1 flow cell on a NovaSeq 6000 sequencing system (Illumina).

### Bioinformatics Analysis

We examined the mRNA and protein expression profiles of Hepa 1-6 tumors treated with PBS, JURV, anti-PD-1 or JURV+anti-PD-1. Three replicates were used to analyze each of the untreated (PBS) and treatment groups. The tumor samples were sequenced on an NGS platform. The files containing the sequencing reads (FASTQ) were then tested for quality control (QC) using MultiQC.¹³⁰ The Cutadapt tool trims the Illumina adapter and low-quality bases at the end. After the quality control, the reads were aligned to a mouse reference genome (mm10/GRCm38) with the HISAT2 aligner¹³¹, followed by counting reads mapped to RefSeq genes with feature counts. We generated the count matrix from the sequence reads using HTSeq-count.¹³² Genes with low counts across the samples affect the false discovery rate, thus reducing the power to detect differentially expressed genes; thus, before identifying differentially expressed genes, we filtered out genes with low expression utilizing a module in the limma-voom tool”. Then, we normalized the counts by using TMM normalization¹³³, a weighted trimmed mean of the log expression proportions used to scale the counts of the samples. Finally, we fitted a linear model in limma to determine differentially expressed genes and expressed data as mean±standard error of the mean. All p values were corrected for multiple comparisons using Benjamini-Hochberg FDR adjustment. After identifying differentially expressed genes, enriched pathways were performed using the Ingenuity Pathway Analyses tool to gain biological insights. The statistical difference between groups was assessed using the nonparametric Mann-Whitney U test R module.

### Integration of Transcriptomics and Proteomics

The limma-normalized transcript expression levels and the normalized protein intensities were integrated using two independent methods. Firstly, the mixOmics package (Omics Data Integration Project R package, version 6.1.1) was implemented to generate heatmaps of the associated DEPs/DEGs as previously described.¹³⁴ Secondly, the MOGSA package was used to generate heatmaps of the top 30 up- or down-regulated DEPs/DEGs between the various groups.¹³⁵

### Blood Chemistry and Cytokines

Blood chemistry analysis was performed with an Abaxis Piccolo Xpress chemistry analyzer (Abaxis) to assess liver toxicity (i.e., aspartate transaminase, alkaline phosphatase, albumin), nephrotoxicity (i.e., creatinine, blood urea nitrogen), and serum electrolytes. Murine type I interferon-beta assay was performed using Mouse IFN beta SimpleStep ELISA® Kit (Cat. #ab252363; Abcam).

### TUNEL Assay Immunohistochemistry

Tumor tissue sections were subjected to the terminal deoxynucleotidyl transferase deoxyuridine triphosphate nick-end labeling (TUNEL) assay using the In Situ Cell Death Detection Kit (Roche Diagnostics, Indianapolis, IN) according to the manufacturer's protocol. After staining, cells were counterstained with 4′,6-diamidino-2-phenylindol (DAPI) to visualize cell nuclei, mounted under cover slips with Prolong® Antifade kit (Invitrogen, Carlsbad, CA) and acquired using the Olympus IX-81 inverted microscope (Olympus America, Center Valley, PA) equipped with Hamamatsu ORCA-ER monochrome camera (Hamamatsu Photonics K.K., Hamamatsu City, Japan). Image analysis was performed using SlideBook 6.2 software. For quantification, 10 independent fields of view were collected per each well (each n) and mean optical density (MOD) or area of colocalization in pixels were recorded for Fluorescein (TUNEL) channel.

### Statistical Analysis

All values were expressed as the mean±standard error of mean, and the results were analyzed by one-way analysis of variance followed by the Tukey test or Benjamini-Hochberg FDR adjustment for multiple comparisons and t test to compare group means. Kaplan-Meier method for survival, using statistical software in GraphPad Prism, version 8 (GraphPad Software). Ap value less than 0.05 was considered statistically significant.

### REFERENCES

1. Getts, D. R., Chastain, E. M., Terry, R. L. & Miller, S. D. Virus infection, antiviral immunity, and autoimmunity. Immunol Rev 255, 197-209 (2013).
2. Dan, J. M., et al. Immunological memory to SARS-CoV-2 assessed for up to 8 months after infection. Science 371(2021).
3. Cook, J., et al. Clinical activity of single-dose systemic oncolytic VSV virotherapy in patients with relapsed refractory T-cell lymphoma. Blood Adv 6, 3268-3279 (2022).
4. Nagalo, B. M., et al. Characterization of Morreton Virus (MORV) as a Novel Oncolytic Virotherapy Platform for Liver Cancers. bioRxiv, 2022.2003.2010.483848 (2022).
5. Travassos da Rosa, A. P., et al. Two new rhabdoviruses (Rhabdoviridae) isolated from birds during surveillance for arboviral encephalitis, northeastern United States. Emerg Infect Dis 8, 614-618 (2002).
6. Dietzgen, R. G., Kondo, H., Goodin, M. M., Kurath, G. & Vasilakis, N. The family Rhabdoviridae: mono- and bipartite negative-sense RNA viruses with diverse genome organization and common evolutionary origins. Virus Res 227, 158-170 (2017).
7. Nagalo, B. M., et al. Characterization of Morreton virus as an oncolytic virotherapy platform for liver cancers. Hepatology (2022).
8. Zhang, Y. S., et al. Bioprinting the Cancer Microenvironment. ACS Biomater Sci Eng 2, 1710-1721 (2016).
9. McNab, F., Mayer-Barber, K., Sher, A., Wack, A. & O'Garra, A. Type I interferons in infectious disease. Nat Rev Immunol 15, 87-103 (2015).
10. Li, S. F., et al. Type I Interferons: Distinct Biological Activities and Current Applications for Viral Infection. Cell Physiol Biochem 51, 2377-2396 (2018).
11. Matveeva, O. V. & Chumakov, P. M. Defects in interferon pathways as potential biomarkers of sensitivity to oncolytic viruses. Rev Med Virol 28, e2008 (2018).
12. Critchley-Thorne, R. J., et al. Impaired interferon signaling is a common immune defect in human cancer. Proc Natl Acad Sci USA 106, 9010-9015 (2009).
13. Naumenko, V. A., Stepanenko, A. A., Lipatova, A. V., Vishnevskiy, D. A. & Chekhonin, V. P. Infection of non-cancer cells: A barrier or support for oncolytic virotherapy? Mol Ther Oncolytics 24, 663-682 (2022).
14. Dumbauld, C., et al. Multi-modal Efficacy of a Chimeric Vesiculovirus Expressing the Morreton Glycoprotein in Sarcoma. bioRxiv, 2022.2009.2018.508434 (2022).
15. Publicover, J., Ramsburg, E., Robek, M. & Rose, J. K. Rapid pathogenesis induced by a vesicular stomatitis virus matrix protein mutant: viral pathogenesis is linked to induction of tumor necrosis factor alpha. J Virol 80, 7028-7036 (2006).
16. Fensterl, V., et al. Interferon-induced Ifit2/ISG54 protects mice from lethal VSV neuropathogenesis. PLoS Pathog 8, e1002712 (2012).
17. van den Pol, A. N., Dalton, K. P. & Rose, J. K. Relative neurotropism of a recombinant rhabdovirus expressing a green fluorescent envelope glycoprotein. J Virol 76, 1309-1327 (2002).
18. Ayala-Breton, C., Barber, G. N., Russell, S. J. & Peng, K. W. Retargeting vesicular stomatitis virus using measles virus envelope glycoproteins. Hum Gene Ther 23, 484-491 (2012).
19. Bourgeois-Daigneault, M. C., et al. Oncolytic vesicular stomatitis virus expressing interferon-gamma has enhanced therapeutic activity. Mol Ther Oncolytics 3, 16001 (2016).
20. Dold, C., et al. Application of interferon modulators to overcome partial resistance of human ovarian cancers to VSV-GP oncolytic viral therapy. Mol Ther Oncolytics 3, 16021 (2016).
21. Hirche, C., et al. Systemic Virus Infections Differentially Modulate Cell Cycle State and Functionality of Long-Term Hematopoietic Stem Cells In Vivo. Cell Rep 19, 2345-2356 (2017).
22. Naghavi, M. H. & Walsh, D. Microtubule Regulation and Function during Virus Infection. J Virol 91(2017).
23. Guo, X., et al. Respiratory Syncytial Virus Infection Upregulates NLRC5 and Major Histocompatibility Complex Class I Expression through RIG-I Induction in Airway Epithelial Cells. J Virol 89, 7636-7645 (2015).
24. Ranjan, P., et al. NLRC5 interacts with RIG-I to induce a robust antiviral response against influenza virus infection. Eur J Immunol 45, 758-772 (2015).
25. Morandi, F. & Pistoia, V. Interactions between HLA-G and HLA-E in Physiological and Pathological Conditions. Front Immunol 5, 394 (2014).
26. Papuchova, H., et al. Endometrial HLA-F expression is influenced by genotypes and correlates differently with immune cell infiltration in IVF and recurrent implantation failure patients. Hum Reprod (2022).
27. Naranjo, N. M., Salem, I., Harris, M. A. & Languino, L. R. IFIT3 (interferon induced protein with tetratricopeptide repeats 3) modulates STAT1 expression in small extracellular vesicles. Biochem J 478, 3905-3921 (2021).
28. Sasaki, M., Sato, Y. & Nakanuma, Y. Interferon-induced protein with tetratricopeptide repeats 3 may be a key factor in primary biliary cholangitis. Sci Rep 11, 11413 (2021).
29. Iwasaki-Hozumi, H., Chagan-Yasutan, H., Ashino, Y. & Hattori, T. Blood Levels of Galectin-9, an Immuno-Regulating Molecule, Reflect the Severity for the Acute and Chronic Infectious Diseases. Biomolecules 11(2021).
30. Lin, T., et al. Hemopexin in severe inflammation and infection: mouse models and human diseases. Crit Care 19, 166 (2015).
31. Kimura, I., Nakayama, Y., Zhao, Y., Konishi, M. & Itoh, N. Neurotrophic effects of neudesin in the central nervous system. Front Neurosci 7, 111 (2013).
32. Novais, A., et al. Neudesin is involved in anxiety behavior: structural and neurochemical correlates. Front Behav Neurosci 7, 119 (2013).
33. Ohta, H., Kimura, I., Konishi, M. & Itoh, N. Neudesin as a unique secreted protein with multi-functional roles in neural functions, energy metabolism, and tumorigenesis. Front Mol Biosci 2, 24 (2015).
34. Lu, G., et al. ISG15 enhances the innate antiviral response by inhibition of IRF-3 degradation. Cell Mol Biol (Noisy-le-grand) 52, 29-41 (2006).
35. Lai, J. H., et al. Mitochondrial CMPK2 mediates immunomodulatory and antiviral activities through IFN-dependent and IFN-independent pathways. iScience 24, 102498 (2021).
36. Zhao, P., et al. Inhibition of rabies virus replication by interferon-stimulated gene 15 and its activating enzyme UBA7. Infect Genet Evol 56, 44-53 (2017).
37. Hamid, O., Ismail, R. & Puzanov, I. Intratumoral Immunotherapy-Update 2019. Oncologist 25, e423-e438 (2020).
38. Durham, N. M., et al. Oncolytic VSV Primes Differential Responses to Immuno-oncology Therapy. Mol Ther 25, 1917-1932 (2017).
39. Jaggi, U., et al. Increased phagocytosis in the presence of enhanced M2-like macrophage responses correlates with increased primary and latent HSV-1 infection. PLoS Pathog 16, e1008971 (2020).
40. Lin, H., et al. Host expression of PD-L1 determines efficacy of PD-L1 pathway blockade-mediated tumor regression. J Clin Invest 128, 805-815 (2018).
41. Tang, H., et al. PD-L1 on host cells is essential for PD-L1 blockade-mediated tumor regression. J Clin Invest 128, 580-588 (2018).
42. Kamphorst, A. O., et al. Proliferation of PD-1+ CD8 T cells in peripheral blood after PD-1-targeted therapy in lung cancer patients. Proc Natl Acad Sci USA 114, 4993-4998 (2017).
43. Galli, G., et al. Innate immune responses support adaptive immunity: NKT cells induce B cell activation. Vaccine 21 Suppl 2, S48-54 (2003).
44. Kim, Y., et al. Complex role of NK cells in regulation of oncolytic virus-bortezomib therapy. Proc Natl Acad Sci USA 115, 4927-4932 (2018).
45. Law, C. W., Chen, Y., Shi, W. & Smyth, G. K. voom: Precision weights unlock linear model analysis tools for RNA-seq read counts. Genome Biol 15, R29 (2014).
46. Baghel, K. S., et al. Macrophages promote matrix protrusive and invasive function of breast cancer cells via MIP-1beta dependent upregulation of MYO3A gene in breast cancer cells. Oncoimmunology 5, e1196299 (2016).
47. Palucka, K. & Banchereau, J. Cancer immunotherapy via dendritic cells. Nat Rev Cancer 12, 265-277 (2012).
48. Liu, R., et al. TRIM67 promotes NFkappaB pathway and cell apoptosis in GA13315 treated lung cancer cells. Mol Med Rep 20, 2936-2944 (2019).
49. Rodrigues, E. & Macauley, M. S. Hypersialylation in Cancer: Modulation of Inflammation and Therapeutic Opportunities. Cancers (Basel) 10(2018).
50. Nishida, N. Role of Oncogenic Pathways on the Cancer Immunosuppressive Microenvironment and Its Clinical Implications in Hepatocellular Carcinoma. Cancers (Basel) 13(2021).
51. Weigert, A., Olesch, C. & Brune, B. Sphingosine-1-Phosphate and Macrophage Biology-How the Sphinx Tames the Big Eater. Front Immunol 10, 1706 (2019).
52. Yang, B., Chen, J. & Teng, Y. TNPO1-Mediated Nuclear Import of FUBP1 Contributes to Tumor Immune Evasion by Increasing NRP1 Expression in Cervical Cancer. J Immunol Res 2021, U.S. Pat. No. 9,994,004 (2021).
53. Wang, Y., et al. CD8+ T Cell Co-Expressed Genes Correlate With Clinical Phenotype and Microenvironments of Urothelial Cancer. Front Oncol 10, 553399 (2020).
54. Caltabiano, R., et al. Macrophage Migration Inhibitory Factor (MIF) and Its Homologue d-Dopachrome Tautomerase (DDT) Inversely Correlate with Inflammation in Discoid Lupus Erythematosus. Molecules 26(2021).
55. Xiong, D., et al. Immunogenomic Landscape Contributes to Hyperprogressive Disease after Anti-PD-1 Immunotherapy for Cancer. iScience 9, 258-277 (2018).
56. Hu, X., et al. Knockdown of SLCO4C1 inhibits cell proliferation and metastasis in endometrial cancer through inactivating the PI3K/Akt signaling pathway. Oncol Rep 43, 919-929 (2020).
57. Clifford, C., et al. Multi-omics in high-grade serous ovarian cancer: Biomarkers from genome to the immunome. Am J Reprod Immunol 80, e12975 (2018).
58. Keppler, D. Towards novel anti-cancer strategies based on cystatin function. Cancer Lett 235, 159-176 (2006).
59. Rozenblit, M., et al. Transcriptomic profiles conducive to immune-mediated tumor rejection in human breast cancer skin metastases treated with Imiquimod. Sci Rep 9, 8572 (2019).
60. Liu, X., et al. Tropomodulin1 Expression Increases Upon Maturation in Dendritic Cells and Promotes Their Maturation and Immune Functions. Front Immunol 11, 587441 (2020).
61. Betzler, A. C., et al. NF-kappaB and Its Role in Checkpoint Control. Int J Mol Sci 21(2020).
62. Takahashi, M., et al. The tumor suppressor kinase DAPK3 drives tumor-intrinsic immunity through the STING-IFN-beta pathway. Nat Immunol 22, 485-496 (2021).
63. Oshima, S., et al. Identification of Tumor Suppressive Genes Regulated by miR-31-5p and miR-31-3p in Head and Neck Squamous Cell Carcinoma. Int J Mol Sci 22(2021).
64. Suefuji, Y., et al. Expression of SART3 antigen and induction of CTLs by SART3-derived peptides in breast cancer patients. Br J Cancer 84, 915-919 (2001).
65. Huang, H., et al. ELTD1 deletion reduces vascular abnormality and improves T-cell recruitment after PD-1 blockade in glioma. Neuro Oncol 24, 398-411 (2022).
66. Harris, R. E. Cyclooxygenase-2 (cox-2) and the inflammogenesis of cancer. Subcell Biochem 42, 93-126 (2007).
67. Colli, L. M., et al. Altered regulation of DPF3, a member of the SWI/SNF complexes, underlies the 14q24 renal cancer susceptibility locus. Am J Hum Genet 108, 1590-1610 (2021).
68. Lakshmanachetty, S., Balaiya, V., High, W. A. & Koster, M. I. Loss of TP63 Promotes the Metastasis of Head and Neck Squamous Cell Carcinoma by Activating MAPK and STAT3 Signaling. Mol Cancer Res 17, 1279-1293 (2019).
69. Ascierto, M. L., et al. Transcriptional Mechanisms of Resistance to Anti-PD-1 Therapy. Clin Cancer Res 23, 3168-3180 (2017).
70. Sun, Y., et al. USH2A Mutation is Associated With Tumor Mutation Burden and Antitumor Immunity in Patients With Colon Adenocarcinoma. Front Genet 12, 762160 (2021).
71. Orentas, R. J., et al. Paired Expression Analysis of Tumor Cell Surface Antigens. Front Oncol7, 173 (2017).
72. Fang, Q. & Chen, H. Development of a Novel Autophagy-Related Prognostic Signature and Nomogram for Hepatocellular Carcinoma. Front Oncol 10, 591356 (2020).
73. MacLean Scott, E., et al. Activation of Th2 cells downregulates CRTh2 through an NFAT1 mediated mechanism. PLoS One 13, e0199156 (2018).
74. Cheung, A. K., et al. Cysteine-rich intestinal protein 2 (CRIP2) acts as a repressor of NF-kappaB-mediated proangiogenic cytokine transcription to suppress tumorigenesis and angiogenesis. Proc Natl Acad Sci USA 108, 8390-8395 (2011).
75. Zeng, Q., et al. Identification of TDP-43 as an oncogene in melanoma and its function during melanoma pathogenesis. Cancer Biol Ther 18, 8-15 (2017).
76. Kumar, R., et al. Role of MAPK/MNK1 signaling in virus replication. Virus Res 253, 48-61(2018).
77. Roger, T., David, J., Glauser, M. P. & Calandra, T. MIF regulates innate immune responses through modulation of Toll-like receptor 4. Nature 414, 920-924 (2001).
78. Lemos de Matos, A., Franco, L. S. & McFadden, G. Oncolytic Viruses and the Immune System: The Dynamic Duo. Mol Ther Methods Clin Dev 17, 349-358 (2020).
79. Oseledchyk, A., et al. Lysis-independent potentiation of immune checkpoint blockade by oncolytic virus. Oncotarget 9, 28702-28716 (2018).
80. Liang, T., et al. FAM46B inhibits cell proliferation and cell cycle progression in prostate cancer through ubiquitination of beta-catenin. Exp Mol Med 50, 1-12 (2018).
81. Shirogane, T., Jin, J., Ang, X. L. & Harper, J. W. SCFbeta-TRCP controls clock-dependent transcription via casein kinase 1-dependent degradation of the mammalian period-1 (Per1) protein. J Biol Chem 280, 26863-26872 (2005).
82. Shen, T. & Huang, S. The role of Cdc25A in the regulation of cell proliferation and apoptosis. Anticancer Agents Med Chem 12, 631-639 (2012).
83. Sobierajska, K., et al. TUBB4B Downregulation Is Critical for Increasing Migration of Metastatic Colon Cancer Cells. Cells 8(2019).
84. Huisman, S. A., et al. Colorectal liver metastases with a disrupted circadian rhythm phase shift the peripheral clock in liver and kidney. Int J Cancer 136, 1024-1032 (2015).
85. Arjona, A. & Sarkar, D. K. Circadian oscillations of clock genes, cytolytic factors, and cytokines in rat NK cells. J Immunol 174, 7618-7624 (2005).
86. Cadenas, C., et al. Loss of circadian clock gene expression is associated with tumor progression in breast cancer. Cell Cycle 13, 3282-3291 (2014).
87. Sobhani, N., Fassl, A., Mondani, G., Generali, D. & Otto, T. Targeting Aberrant FGFR Signaling to Overcome CDK4/6 Inhibitor Resistance in Breast Cancer. Cells 10(2021).
88. Touat, M., Ileana, E., Postel-Vinay, S., Andre, F. & Soria, J. C. Targeting FGFR Signaling in Cancer. Clin Cancer Res 21, 2684-2694 (2015).
89. Davids, M. S., et al. Response to a novel multitargeted tyrosine kinase inhibitor pazopanib in metastatic Merkel cell carcinoma. J Clin Oncol 27, e97-100 (2009).
90. Kim, S. T., et al. Pazopanib, a novel multitargeted kinase inhibitor, shows potent in vitro antitumor activity in gastric cancer cell lines with FGFR2 amplification. Mol Cancer Ther 13, 2527-2536 (2014).
91. Sonpavde, G. & Hutson, T. E. Pazopanib: a novel multitargeted tyrosine kinase inhibitor. Curr Oncol Rep 9, 115-119 (2007).
92. Jo, J. C., et al. Targeting FGFR Pathway in Human Hepatocellular Carcinoma: Expressing pFGFR and pMET for Antitumor Activity. Mol Cancer Ther 14, 2613-2622 (2015).
93. Ahn, D. H., et al. A pilot study of Pan-FGFR inhibitor ponatinib in patients with FGFR-altered advanced cholangiocarcinoma. Invest New Drugs 40, 134-141 (2022).
94. Knutson, K. L. & Disis, M. L. Tumor antigen-specific T helper cells in cancer immunity and immunotherapy. Cancer Immunol Immunother 54, 721-728 (2005).
95. Pan, Q. Z., et al. Annexin A3 as a potential target for immunotherapy of liver cancer stem-like cells. Stem Cells 33, 354-366 (2015).
96. Elgundi, Z., et al. Cancer Metastasis: The Role of the Extracellular Matrix and the Heparan Sulfate Proteoglycan Perlecan. Front Oncol 9, 1482 (2019).
97. Leung, T., et al. Cytochrome P450 2E1 (CYP2E1) regulates the response to oxidative stress and migration of breast cancer cells. Breast Cancer Res 15, R107 (2013).
98. Li, H., et al. Ferroptosis-related gene signature predicts the prognosis in Oral squamous cell carcinoma patients. BMC Cancer 21, 835 (2021).
99. Ma, G., et al. LCP1 triggers mTORC2/AKT activity and is pharmacologically targeted by enzastaurin in hypereosinophilia. Mol Carcinog 59, 87-103 (2020).
100. Liu, W., Li, L., Ye, H., Tao, H. & He, H. Role of COL6A3 in colorectal cancer. Oncol Rep 39, 2527-2536 (2018).
101. Datta, M. W., et al. Perlecan, a candidate gene for the CAPB locus, regulates prostate cancer cell growth via the Sonic Hedgehog pathway. Mol Cancer 5, 9 (2006).
102. Heske, C. M. Beyond Energy Metabolism: Exploiting the Additional Roles of NAMPT for Cancer Therapy. Front Oncol 9, 1514 (2019).
103. Chen, G., Wang, H., Xie, S., Ma, J. & Wang, G. STAT1 negatively regulates hepatocellular carcinoma cell proliferation. Oncol Rep 29, 2303-2310 (2013).
104. Satelli, A. & Li, S. Vimentin in cancer and its potential as a molecular target for cancer therapy. Cell Mol Life Sci 68, 3033-3046 (2011).
105. Fischer, A., Notarangelo, L. D., Neven, B., Cavazzana, M. & Puck, J. M. Severe combined immunodeficiencies and related disorders. Nat Rev Dis Primers 1, 15061 (2015).
106. Bishnoi, S., Tiwari, R., Gupta, S., Byrareddy, S. N. & Nayak, D. Oncotargeting by Vesicular Stomatitis Virus (VSV): Advances in Cancer Therapy. Viruses 10(2018).
107. Koyama, A. H. Induction of apoptotic DNA fragmentation by the infection of vesicular stomatitis virus. Virus Res 37, 285-290 (1995).
108. Rogers, C., et al. Cleavage of DFNA5 by caspase-3 during apoptosis mediates progression to secondary necrotic/pyroptotic cell death. Nat Commun 8, 14128 (2017).
109. Kubo, T., et al. Oncolytic vesicular stomatitis virus administered by isolated limb perfusion suppresses osteosarcoma growth. J Orthop Res 29, 795-800 (2011).
110. Le Boeuf, F., et al. Sensitivity of cervical carcinoma cells to vesicular stomatitis virus-induced oncolysis: potential role of human papilloma virus infection. Int J Cancer 131, E204-215 (2012).
111. Ahmed, M., Puckett, S. & Lyles, D. S. Susceptibility of breast cancer cells to an oncolytic matrix (M) protein mutant of vesicular stomatitis virus. Cancer Gene Ther 17, 883-892 (2010).
112. Wollmann, G., Davis, J. N., Bosenberg, M. W. & van den Pol, A. N. Vesicular stomatitis virus variants selectively infect and kill human melanomas but not normal melanocytes. J Virol 87, 6644-6659 (2013).
113. Ebert, O., et al. Oncolytic vesicular stomatitis virus for treatment of orthotopic hepatocellular carcinoma in immune-competent rats. Cancer Res 63, 3605-3611 (2003).
114. Nagalo, B. M., et al. Oncolytic Virus with Attributes of Vesicular Stomatitis Virus and Measles Virus in Hepatobiliary and Pancreatic Cancers. Mol Ther Oncolytics 18, 546-555 (2020).
115. Cary, Z. D., Willingham, M. C. & Lyles, D. S. Oncolytic vesicular stomatitis virus induces apoptosis in U87 glioblastoma cells by a type II death receptor mechanism and induces cell death and tumor clearance in vivo. J Virol 85, 5708-5717 (2011).
116. Zhang, Y. & Nagalo, B. M. Immunovirotherapy Based on Recombinant Vesicular Stomatitis Virus: Where Are We? Front Immunol 13, 898631 (2022).
117. Quiroz, E., Moreno, N., Peralta, P. H. & Tesh, R. B. A human case of encephalitis associated with vesicular stomatitis virus (Indiana serotype) infection. Am J Trop Med Hyg 39, 312-314 (1988).
118. Patterson, W. C., Mott, L. O. & Jenney, E. W. A study of vesicular stomatitis in man. J Am Vet Med Assoc 133, 57-62 (1958).
119. Inoue, H. & Tani, K. Multimodal immunogenic cancer cell death as a consequence of anticancer cytotoxic treatments. Cell Death Differ 21, 39-49 (2014).
120. Antoniades, J., Brady, L. W. & Lightfoot, D. A. Lymphangiographic demonstration of the abscopal effect in patients with malignant lymphomas. Int J Radiat Oncol Biol Phys 2, 141-147 (1977).
121. Lakshmanagowda, P. B., et al. Abscopal effect in a patient with chronic lymphocytic leukemia during radiation therapy: a case report. Cases J2, 204 (2009).
122. Cotter, S. E., et al. Abscopal effect in a patient with metastatic Merkel cell carcinoma following radiation therapy: potential role of induced antitumor immunity. Arch Dermatol 147, 870-872 (2011).
123. Takaya, M., et al. Abscopal effect of radiation on toruliform para-aortic lymph node metastases of advanced uterine cervical carcinoma—a case report. Anticancer Res 27, 499-503 (2007).
124. Ohmatsu, K., et al. Abscopal complete regression of hepatocellular carcinoma with multiple pleural metastases. Int Cancer Conf J 10, 54-58 (2021).
125. Randrian, V., et al. FFCD 1709-SIRTCI phase II trial: Selective internal radiation therapy plus Xelox, Bevacizumab and Atezolizumab in liver-dominant metastatic colorectal cancer. Dig Liver Dis 54, 857-863 (2022).
126. Powers, T. W., et al. MALDI imaging mass spectrometry profiling of N-glycans in formalin-fixed paraffin embedded clinical tissue blocks and tissue microarrays. PLoS One 9, e106255 (2014).
127. Casadonte, R. & Caprioli, R. M. Proteomic analysis of formalin-fixed paraffin-embedded tissue by MALDI imaging mass spectrometry. Nat Protoc 6, 1695-1709 (2011).
128. Wisniewski, J. R., Zougman, A., Nagaraj, N. & Mann, M. Universal sample preparation method for proteome analysis. Nat Methods 6, 359-362 (2009).
129. Nesvizhskii, A. I., Keller, A., Kolker, E. & Aebersold, R. A statistical model for identifying proteins by tandem mass spectrometry. Anal Chem 75, 4646-4658 (2003).
130. Ewels, P., Magnusson, M., Lundin, S. & Kaller, M. MultiQC: summarize analysis results for multiple tools and samples in a single report. Bioinformatics 32, 3047-3048 (2016).
131. Kim, D., Langmead, B. & Salzberg, S. L. HISAT: a fast spliced aligner with low memory requirements. Nat Methods 12, 357-360 (2015).
132. Anders, S., Pyl, P. T. & Huber, W. HTSeq—a Python framework to work with high-throughput sequencing data. Bioinformatics 31, 166-169 (2015).
133. Robinson, M. D. & Oshlack, A. A scaling normalization method for differential expression analysis of RNA-seq data. Genome Biol 11, R25 (2010).
134. Rohart, F., Gautier, B., Singh, A. & Le Cao, K. A. mixOmics: An R package for 'omics feature selection and multiple data integration. PLoS Comput Biol 13, e1005752 (2017).
135. Meng, C., et al. MOGSA: Integrative Single Sample Gene-set Analysis of Multiple Omics Data. Mol Cell Proteomics 18, S153-S168 (2019).

1. A construct comprising a promoter operably linked to a polynucleotide encoding a full length antisense Jurona virus genome and allowing production of a negative sense viral genome when transfected into mammalian cells, wherein the polynucleotide encoding the Jurona virus genome comprises SEQ ID NOs: 1-5 or wherein the polynucleotide encoding the Jurona virus genome comprises SEQ ID NO: 12 (JURV-XN-2) or a sequence having at least 95% identity to SEQ ID NO: 12.

2. The construct of claim 1, wherein the promoter is a T7 promoter.

3. (canceled)

4. The construct of claim 1, wherein when the polynucleotide encoding the Jurona virus genome comprises SEQ ID NOs: 1-5, the polynucleotide encoding the Jurona virus genome further comprises a leader sequence of SEQ ID NO: 6 and/or a trailer sequence of SEQ ID NO: 7.

5. The construct of claim 1, wherein when the polynucleotide encoding the Jurona virus genome comprises SEQ ID NOs: 1-5, the polynucleotide encoding the Jurona virus genome further comprises at least one of SEQ ID NOs: 8-11, 21, and 22 as intergenic regions.

6. (canceled)

7. The construct of claim 1, wherein the polynucleotide encoding the Jurona virus genome further comprises a heterologous polynucleotide capable of encoding a polypeptide not natively associated with Jurona virus.

8. The construct of claim 7, wherein the polypeptide is a reporter polypeptide.

9. The construct of claim 8, wherein the reporter polypeptide is a fluorescent protein.

10. The construct of claim 9, wherein the polynucleotide comprises SEQ ID NO: 13 (JURV-eGFP) or a sequence having at least 95% identity to SEQ ID NO: 13.

11-17. (canceled)

18. A cell comprising the construct of claim 1.

19. (canceled)

20. An infectious particle comprising a Jurona virus genome comprising a negative sense RNA of SEQ ID NO: 12 or having 95% identity to SEQ ID NO:12.

21. An infectious particle made by transfecting cells with the construct of claim 1.

22. A pharmaceutical composition comprising the infectious particle of claim 21 and a pharmaceutically acceptable carrier or excipient.

23.-56. (canceled)

57. A kit comprising the construct of claim 1.

58. The kit of claim 57, further comprising an immune checkpoint inhibitor selected from the group consisting of inhibitors of PD-1, inhibitors of PD-L1, inhibitors of CTLA-4, and inhibitors of LAG-3.

59. The kit of claim 57, further comprising an inhibitor of IFN-α.

60. The kit of claim 57, further comprising a receptor tyrosine kinase inhibitor.

61. The kit of claim 60, wherein the receptor tyrosine kinase inhibitor is pazopanib.


SEQUENCE LISTING (22 sequences)

SEQ ID NO: 1 - DNA, 1269 bp, Jurona virus
  source 1..1269: mol_type = genomic DNA
atgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttag

SEQ ID NO: 2 - DNA, 885 bp, Jurona virus
  source 1..885: mol_type = genomic DNA
atgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataa

SEQ ID NO: 3 - DNA, 678 bp, Jurona virus
  source 1..678: mol_type = genomic DNA
atgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaa

SEQ ID NO: 4 - DNA, 1575 bp, Jurona virus
  source 1..1575: mol_type = genomic DNA
atggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatga

SEQ ID NO: 5 - DNA, 6273 bp, Jurona virus
  source 1..6273: mol_type = genomic DNA
atggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataa

SEQ ID NO: 6 - DNA, 54 bp, synthetic construct
  source 1..54: mol_type = other DNA
acgaagacaaacaaaccattattatcattaaaaggctcggtaacagtaatcaaa

SEQ ID NO: 7 - DNA, 252 bp, synthetic construct
  source 1..252: mol_type = other DNA
tggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataa

SEQ ID NO: 8 - DNA, 109 bp, Jurona virus
  source 1..109: mol_type = genomic DNA
acaaaactgcagaatccttctagtatgcgcgacgatggcattggatatcttattttttgaaattgctcaacttattaacctgttatgaaaaaaactaacagacatcaac

SEQ ID NO: 9 - DNA, 78 bp, Jurona virus
  source 1..78: mol_type = genomic DNA
tctacttactgcatcgcattgttcatgggtatgaaaaaaactaacagtgatccacactgacccgctgtctccccgaag

SEQ ID NO: 10 - DNA, 32 bp, Jurona virus
  source 1..32: mol_type = genomic DNA
aagatctatgaaaaaaactaacagtgatcaaa

SEQ ID NO: 11 - DNA, 113 bp, Jurona virus
  source 1..113: mol_type = genomic DNA
ccatgttgaagtcaatcatttctcctataacctcggctgtttgtgaaatcatcatatgtgatatgaaaaaaactaacagtcatcgctagctatgaaaaaaactaacagtcatc

SEQ ID NO: 12 - DNA, 11318 bp, synthetic construct
  source 1..11318: mol_type = other DNA
acgaagacaaacaaaccattattatcattaaaaggctcggtaacagtaatcaaaatgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttagacaaaactgcagaatccttctagtatgcgcgacgatggcattggatatcttattttttgaaattgctcaacttattaacctgttatgaaaaaaactaacagacatcaacatgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataatctacttactgcatcgcattgttcatgggtatgaaaaaaactaacagtgatccacactgacccgctgtctccccgaagatgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaaaagatctatgaaaaaaactaacagtgatcaaaatggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatgaccatgttgaagtcaatcatttctcctataacctcggctgtttgtgaaatcatcatatgtgatatgaaaaaaactaacagtcatcgctagctatgaaaaaaactaacagtcatcatggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataa

SEQ ID NO: 13 - DNA, 12061 bp, synthetic construct
  source 1..12061: mol_type = other DNA
acgaagacaaacaaaccattattatcattaaaaggctcggtaacagtaatcaaaatgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttagtatgaaaaaaactaacagagatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacaaaactgcagaatccttctagtatgcgcgacgatggcattggatatcttattttttgaaattgctcaacttattaacctgttatgaaaaaaactaacagacatcaacatgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataatctacttactgcatcgcattgttcatgggtatgaaaaaaactaacagtgatccacactgacccgctgtctccccgaagatgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaaaagatctatgaaaaaaactaacagtgatcaaaatggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatgaccatgttgaagtcaatcatttctcctataacctcggctgtttgtgaaatcatcatatgtgatatgaaaaaaactaacagtcatcgctagctatgaaaaaaactaacagtcatcatggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataa

SEQ ID NO: 14 - DNA, 14261 bp, synthetic construct
  source 1..14261: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttagacaaaactgcagaatccttctagtatgcgcgacgatggcattggatatcttattttttgaaattgctcaacttattaacctgttatgaaaaaaactaacagacatcaacatgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataatctacttactgcatcgcattgttcatgggtatgaaaaaaactaacagtgatccacactgacccgctgtctccccgaagatgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaaaagatctatgaaaaaaactaacagtgatcaaaatggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatgaccatgttgaagtcaatcatttctcctataacctcggctgtttgtgaaatcatcatatgtgatatgaaaaaaactaacagtcatcgctagctatgaaaaaaactaacagtcatcatggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 15 - DNA, 15004 bp, synthetic construct
  source 1..15004: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttagtatgaaaaaaactaacagagatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacaaaactgcagaatccttctagtatgcgcgacgatggcattggatatcttattttttgaaattgctcaacttattaacctgttatgaaaaaaactaacagacatcaacatgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataatctacttactgcatcgcattgttcatgggtatgaaaaaaactaacagtgatccacactgacccgctgtctccccgaagatgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaaaagatctatgaaaaaaactaacagtgatcaaaatggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatgaccatgttgaagtcaatcatttctcctataacctcggctgtttgtgaaatcatcatatgtgatatgaaaaaaactaacagtcatcgctagctatgaaaaaaactaacagtcatcatggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 16 - DNA, 4518 bp, synthetic construct
  source 1..4518: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatgtcctctatggtcaagcgaatttctacgggctcaaatgtgttggcagttttgccagcaagtgaggatccagtggagtacccagaggactattttaaggggaatccaggaaaggttaaggtcttcatcagccgaggtacagatctgaacaaattgagacaattcgtttacgagggaatcaagtcaggagatgttagcatctgccacatcaattcctatctctatcaagtcttgaaagggattcaagaagaggctccatctgaatggaaaagctttggagtgcagatagcagtcaagggtcaaaagataggcatttttgatcttttggacgtagacgactacagaggacccatacccgacggcaaatccagcagtggacgaaccgcagatgatgacaaatggctgccgatgctcatactggggctatacagagtgggaagagcaactcaagaagactacaagaaaatgctgatggccaaccttaatgctcagtgcaagctgagaagcaatcaggcggaagaaattgttgatgacacatctgacttttaccaggcctgggggaacgacagtaacttcctaaagattgttgcagccgtggatatgtttttccattatttcaagaaaaatccagaggccaacatcagattcggaaccattgtgtcacgcttcaaagactgcgctgcattaacaacactggcccatttacagaaggtcactggtcttccaatagaagaagtcttctcgtggatcctcactcggtccgtcgaagatgaggtctgcaggatgatgaaaccaggtcaggaaattgaccaagccaactcttttatgccctatctgatagacctgggcctgtccaccaagtcgccttactcatcagtgaagaaccccagtttccatttttggggacaattaactgctttattggtgcaatcttcaagggccaaaaatgcaagagttccagatgacattccgtatgcagaactgacaaaagcagctcagctttttggctttgccataggtcactcctcagacctggaacagagatttgcactggggaacaaggtctacaaaaaggatgagacagatgcggctggatccatgctcgagcctccaacatccaaggatgttgtggaatggcttgcatggtgggatgatgtcggaaatgtccccactccagagatggatgcctttgccaaaagagcagtccatggaatgattgacatccgcactagaactattggagaatacgccagaaaaatattctcttagtggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 17 - DNA, 4134 bp, synthetic construct
  source 1..4134: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatgtctgagctcaagaacgttctaaagaactatccaaatattaatcaaacttttaatgagttgaatgagatggaagagcatgggtccgttttaccaaacagcatcactgaagatgcccctgccgtccagacgcccctctatcattctcttgacatcttgtcagagtcttctgaggaagatctggaagaggagtcagagagcgaggagttggatgaggtccaagtggatgatgagaaggatcagttggaagagattgatgatgacaactttcatgtatctttcaatgagagacccccctggaccgccatgacccagaaaacgatcaatgggaaattgagagtcaacatgtctgctccagaaggtctgaatgagctgcaatttgagcaatgggtctccagtctggagaatttaatgtccttatcaaagtccctcagacttcatgctgccgagttgagcatagtggaggatggggtgcagattgatgaaaaactgagcccctgtttgtctaggacttcatgtttcagggacattcccgaatttaaaagccctatccctgagaagaaaggcaatgagaccgtgactgccacatcatcaccgagtggaagctgtgagagcgagtccagcgatctctccggaattccaaatttcagccgggatgacatcaaagggctcatcgagaaagagttcactctagagaccacagacaaaaaatgcaaggaatacacctcatcactcgcagagctcttcggatccaaagaagcagccctagctttcctgaacgggaatcctgttggccttgatgaatttgtttgtgcaggactaaagaggaagggaatctttaacagaatgaggatcaagtatgtgttaaaacccattttcaaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 18 - DNA, 3927 bp, synthetic construct
  source 1..3927: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatgaagagcttcaaaggaatgtttaagaactataaggaaaagagcaaaatgaaaaaggagatggactgggactctcctcccagttacacagatgtgagaaggggcatctatccgtcagcacctttgttcgggctggatgacgacttgatggagactctcccttcattaggtatccagaacataaaactccaatacaagtgcaatattcaattaagagcagaatttccttttgtttcttattcagaggtggttttggcgttgagccaatgggatgaggagtatcgtggtttccttgggaagaagccattttatcgagctgtcattttgagaacatctaagaacttgaaagctgtgcctctaagcttgactgacggaggccgtccggaatacaatggagaagtacaaggtcagagttccctctaccacagcttagggattattcctcctatgatgtatgtgccggagactttcatgaaggagtggaagactgcaggaaatcgcggagtgctgattgttaaaatttgggtgggagtcacagacactctcgacacgcttgatcccttgctacacccaattgtctttaaatcagagaaggaattgattgagatggccaaagtttttggcctggatgtctccaaagggagggacaataattggataatatcaaaatcttattaatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 19 - DNA, 4824 bp, synthetic construct
  source 1..4824: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatggagtcctcactccatgtccttgtccttgttgtgctaagtgcagacttatgtaggtcagcaattccaattttctttcctagtgaaccgcagttagaatggaaacccgtgctgcccggatcccgttattgtcctcaatcaaatgagatgtctctggatcctgatttgaagaaagccacaatttctgtgaaagtccctattggcgtgaccccatctaagtcagatggttacttgtgtcatggcgccaaatgggtgtctacgtgtgactttcggtggtatggaccaaaatacattactcattcaatacataatctcagaccaactaccaatgactgtgaagacgcaataaagaagtatgaagcgggcacgctcataaatcctggcttccctccagactcctgtgcttatgcaactgtcaccgattcagaacatctggtcattttaatcacacctcatcatgtaggagtcgatgattatcgaggtgcctgggtagatgactcatttccatccggaatctgtgaaacaaatcagtgcgacacaacccataactcgtcaatttggattcctaaggtcaaaacaagacataacatctgtagtcaaacctttgcgaacctatccatgaccatttcttacagagaaggaggggcaatgaagggatctgacttggtgtttcacagcaagtatcatcctcatatgatgggaggtcacacctgcaagatgactttttgcaataagcagggtttgagattacagaatgaggaatggattgagatccccacagaaacgaaagtgggaaatcaagatctgatgaacctgttctcagactgcaagtcaggcctagagattcggtctactcttaggtctgaaggagcgaacaccctcacttgggagacacaaagattgctagactatgccttgtgtcaaaacacttgggataaatttgaggagcaaggggccgtttctgctttagacctcagttatcttgccgccaaggccccagggaagggagtcgcttacaccatgatcaatggcacacttcattctgctccaacgagatatgtcagaatgtggatagagagcccctccatggaggagttgaaggcaaagaaggaatcatcctcaggagtagagacagaaatatggaagcaatggtttcctttcaaagggggcgaaattgggcccaacggattgatccgggcagggaacaggtacaaatttccactctaccttgtcggaatgggaatgttggatgatgagatcaatgcattggagctagggggcccaatagatcacccccagcggtcgcatgcccaggcggctttgggggatgaggaaacactcttctttggggacactggagtgggaaaaaatccggttgagttgattacgggctggttttccagttggaaagaaacaatgatggcaggcattgctgttttcctgtttgtaattattctgtatggagtcctcagatgttttcctaagatatgtctgatttgcaaaaggaggtcaagtcaacgagccaaagacatagagatgcaatatattcccaacaatcaacgtcagtggcgatgatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 20 - DNA, 9522 bp, synthetic construct
  source 1..9522: mol_type = other DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagaattgtaatacgactcactataggacgaagacaaacaaaccattattatcattaaaaggctcggtaacagtaatcaaaatggactgtgaatctgaacagtggggagatcccttatctgaggacaacttcgtttccggacaaattcccaaagaggatcgagttcgaaatcttaactctgttgattacaacctaaattctcctctgatatctgatgacatcaggtacttgatatctcgatacaatgggaaagcggttcctagcatctggaaaaagaaaaattggaatgagactatagacaatctgagagaattaaatttcacccctattcaggtagaaaaaatgcataattggtatggagaatggcttctaaatcatccgagtcacattgagcaggggagggcatttctgtctgatgttgacaaagagagtgagatcacatactgtgtggtcgagtcctttcttaaaggatggactggaacttcatgtgctttcaaaattaaaaatggccctcacatggattatgcctctggattgtgtcaaaaatttcttgacctacataaattgacattgttgatgaatgcatcaacacatactgaggagatgagtttagctaacaccttcaagatcaagagatatttagaagacaagacagtcaaactgcctactataggcaaagtaaaaatgtcaggacaattcttgttattaatcactcaatctgttgttcttgacaggaattttttactgatgatgaaagatgtcattattggccgtttgcagacaatactatccatgataaatagatcagatgatagattttctgataaggatataacagatctcagagctctgtatgctgttggtgacaatatcttgaaggagctgggaaatcaaggatttgacttgataaaaacaattgaaccgatatgcaacttaaaactttccgagatggcaagagagtatcggcctttgattcctgattttcctcatttcagagagcacgtgattaaaacagtgaaggatttatcagaattgaacaagagggtccaaaccctatttgacatgattatgggactcaaaagtctggaggttgtcctggttgtttattcatcctttcgacactggggccatccattcattgattacattgaaggattgaaaaagttgcatcaccaagttactttggagaaagacatagatgaagactatgctaaagcactagcaagtgacttggcacgtatagtcctcaccaaagaatttaatgaaaagaagaggtggagtgtggactatgacaagctaccggcaaatcatgtattcaaagagcatgtaagagacaacacttggccaactcccgcactgatacaggatttcggagataagtggcatgagttgcctttagtccaatgctttgatattccagatttgattgacccatccattatttactcggataaaagtcactcgatgaacagaagagatgtgataaatcacataaaatacaaatctgatcagcctattcccagcaaaaaagtcctcaagacaatgatagaggaaccagcaaccaactggttagaattcctggaaatgatagacaaacacggactagatgaagatgatttaattattgggctcaaagggaaagaacgagagttgaagattgctgggagattcttctcattaatgtcctggaaactacgagaatattttgttatcacagagtatttaatcaagacacactttgtacctcttttttatggacttaccatggcagatgatatgacctctgtaatcaaaaagatgcttgagagttcttccggtcagggtctcaatgattattctgctatatccatagcgaatcacattgactatgaaaagtggaataaccatcagagaaggacatcaaatgaaccggtcttccgagtaatgggtcagttcttgggttacccaaatttaatctcaaggacacatgagttctttgagaaaagtctgatttactacaatggaagaccggatctaatgagagtccgaaatgggattgttgagaactcctcagaagtccgggtctgttggaacggacaggcaggaggcctcgaaggtctaagacagaaagggtggagcatcttaaacctactagtgattcagagggaatcaaaaattcggaacacatctgtcaaagtcttagcccaaggagacaaccaagtcatctgtactcaatacaaaaccaaacaacatcgttcagacattgagcttagaggtgctttggaacaaatgaagtcaaataatcaaactataatggactcgattgagcgggggacgaataaactcggtctattgatcaatcaggatgagaccatgcaatcagccgattatttaaattatgggaaagtccctatttttaggggagtcatcagagggctggaaaccaaaagatggtcccgagttacctgtgtgactaatgatcaattacctacctgtgctaatctcctttcatccgtctccaccaatgctttaaccgtatcacactttagtgtaaaccccatcaacgcaatgatacaatataattatttttcaaacttctcaagactgcttctttacatgcatgatccagccctcagatgttctttgtacgaagacaaataccgacacctccagattgattctctcggatttaagctggcaatagcttacttggatccctcgattgggggtgtttgtggaactgctttaactcggtttttgatccgaagtttccccgatcccgtctctgaaagtctttctttctggaaactgattcatgataatacgaccaaccaaaccttgaaaaacctctcgatacaatttggcaacccgaaaatagctcagttcaagccatctcatatagataaactcctagaggacccaaccagcctgaacatcgcaatgggaatgagccccacgaatttgttgaaaacagaaatcaagaaaaatctgttgagaacacgcaattcaataaaaaacaaaattgtcaaagatgcagtaagttatatacattcagaggatgaaaatttgaggagttttttgtggagcatcaatcccctattccccagatttctcagcgaatttaaatcaggaacattcatgggagtggcatcgagcatcgtcggcttgtttcaaaattccagaactattcgcaacaccttccggagcttcttaagcaatcaaatagatgatttgattgtaaaaagtgagcaaacttctctagaacatctagcgtcttacacagggaaacacgtgacaaaaatatggacatgctcttcatcacattctgatcaattgagaaagaaatcctggggaaggcaagtacttgggactactataccccatcctatcgaaatgcatgggaaaggatccttgaagaccgctacatctacctgttgtcagcacagaacactggactacatatctgttcattgtccgaaggggctgagtaatgttttggacgccagaggagatcttgctgcatatttagggtcaaaaacatcagagtcaacatccatcctgcagccgtgggaaaaagaaagcaagatcccactaatcaagcgagcaacaagattgagagacgccatccattggtttgtggaagccgattcgaaccttgcccgaagcataacaaataacatagtgtcattgacaggagaagactggggatcatccgtgaaggggttcaaaaggacaggctctgcccttcacagatttagcacatcaaggatgagccatggtggatttgctgcacaaagcccagcttgcttgaccagaatgttatcgacaaccgacacaatgagagactactcaaccgaaaatttcgatttcatgttccaagcaagtttgatctactctcaagcatccacatctgttatgttgtatgggaccagcacccctaacaccattcacttccacacggcgtgtaaaaattgcattcggaaaatcgaagaaccctggctggattccccgcgggtgtacaaatcaaaggatgtttcccatgttcttggagggtggcgcaacggacagggggcttggggttcttctattcagcaattaaagcccattccaggcaactgggatgctctgtccgcatctgagaaatcatatcatgtaggtagggctttgggcttcttgtatggagatttaatcggacagtcatcgagccgttctgaggacagttctattttccctctgagtattcaatacaagctgagaggaagaggattcatgagagggattctagatggtttggtgagagctagtgcctgccaagttattcacagaaggagcattgcattgttggtcaaaccggcaaatgcaatttacggtggactgatttttctcattgaacggttgagcaattcttcttgtttcgtcaatctatgcagagacggacctatccgagaagaacttagaagtatccctcacaagattccaacctcttacccaacatcctctatggatatgggtttgaatgtaaggaattacctcaaatatcagtgcaagtcagtggaattggggaaatacaagtcagacatcgaaacactgtggatcttctcggacctaatgtcagtgacattttctggcccattcgctctatcttcgaaaatactgagacatttgtataaaccaaccctgtcaaaaaaggatcgtaccaacttaagaaggttgtcaaacttctcacaaatactgaggtcgaaggaatcctgggatggactagagcaggaatacttaagttcaaaactatcagtttgtgaagaggaagtgcggcacgcatgcaagtttggaatcaagatgatccctctggaaattcctacctatcagtggggggaagaagaagcaggatgggtaactgcattcaaagttgaattgctcccgtatgaagtcccaaagctgttgttgaattgcccacgaaaacagaatccgacaatctctggtatccggcttggacaactcccgacaggagcacactataagattaggtcaatcctcaaagagaaacaaataaaataccgagatttcctctgtggaggagatggatccggcgggatgaccgcggcatgtttgagatacaaccctagaagtcgaggaatattcaacagcatattagaattcgatggatcctcaatgaaagggtcatccccggacccaccaagcgctttagaaacagttagtgacggaaaaaagaggtgtgtaaatgcactcgattgctgggaatacccttccgacctgagtcttaaagagacttggaagtattttaagatcttgattcaagaacattcattgagggttgacttgattgtcttagatatggaggttagggaaattgagatttcagagagaatagagaggcaaattagaaacaacatatatgacatactgacaacgaatggaacgctaatatacaaaacttatggatcagtgatttcatccaacaaggagaacagtttgacgaggcttggacccctgtttgaagaagtggagttgttgcaaaccgaatatagcagttcatccacatctgaaatatatcttgtcggtcggaaattgaaaaattttgtcgacatgaagtggatagattggacatccctacaatatcattgggacaaactcttttgttttagaagcccatcagatgagtttaagcgagccaaatccttattgtctaaagagcacttaaaaggaatacctcgatcgtttctaccagaccccctagtcaatctcgaaacactcatgcaaatcagtggtatcccatctgggattgcgcaccagctgtgtttggattttaaagcgcaatcagtgtcagggataaccgcatccctgttgataatgtccctatcttcacaatttgctattgacactacaaggcgagtcacttctttagttcctccttccgatggtcgtttgcacaagatggcctgctgcattgtaggggtcagtttatggacttctttaaggtatgatgacctattgttaaacaaaacctgtgtagatagcatcaatcgatcgtttccggtaagaatctcctgggaaaacggattcaatcaatggaacacgtatggaataggactcccgaaggacatcagaatatctgacggatctgcaatcctaggcaattggattcgaggtatggagttaatgaacttgagtggggagaattttgacatcaaaaagtttgacaagatctcgtgtaaatatatcaagaccctcaattatcaacatgtgaaaaagaagacgggtctgaagaactggatcaaaggaaaggtctcgaaagaagatatgtcattgatgcgattgacatcaggagacataccatcagaacattgggtcgaataatggcaagatttataaggcaattggagggacgtggcccatcagtttccgtttgaaaaaaaccaatggcatgggttatataccatgtgtacgttttaagagtgtaaatgatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 21 - DNA, 40 bp, Jurona virus
  source 1..40: mol_type = genomic DNA
ggtatgaaaaaaactattcaacagtgatccacactgaccc

SEQ ID NO: 22 - DNA, 40 bp, Jurona virus
  source 1..40: mol_type = genomic DNA
ccttatgaaaaaaactaacagaaatcattctgtttcgtca